SEQUENCE LISTING (60 sequences)

SEQ ID NO: 1 - protein, 637 aa, Unknown
  Spongosorites species
NYGAIETTDNNGNTKSNFVEFTNPPQCTAPDTVYAKGADFVPNRTVDIYITKQPDNQNFIPLPQPQNPTNPGQFKQVGESYQLTDVRTTGKSTHNIDANGNIANTAVWVQPTPNQQYNLIVDVDRDGYFTNSIDIVDAFNTQGFDVDPCLRKRLAASLSDGIPAGDGTRDFVKPPKIFISGDWFPQGSARIYIIIYDKNFDYNGNDSKVLTDVRGTYDTITVGENHKFTGLEWSSPHVGNYNIIFDANSDGVYKKSDGDIVDHINEIGFAMLESYPHHSHIVDLDDNANQREDFNSGIAKNVYVLATNLPNPSTIVDVYVISEKLLKLNNPGWTTWQQSSNVPLKESAAPTNNGDRLHPTWVTPQKTVFGSVWHMPGDTFDPPYINYYGKKFTIVIDVNRDGKYDPSIDMVDTHDIGDMTDWFITQGHHDLSPTSGNGQPAVSEYKEYLNSKLDLENPLDNTNNYDAATEAASAQYLCRTNLPINLFENVIELGAQTGFIMFNPDEYYAGRDLNSGRHIYDAVDFDGLEINDGYTSVISAKEMTLNRLGVGQNSNLLTCGADTVTIDGMSTQKDSTTNIVAKTISHKSNISLEADTCTHYTHNLYFESDTNILVKAIESPFAWIIDVGDRILPGTGC

SEQ ID NO: 2 - protein, 305 aa, Unknown
  Spongosorites species
RQIDIASWNTFDFGGKFGGVCKANDPTILQAMTDVISQHDVILVQEIANVTNPCNPANIFSGFNNLCSNNYLGGLGYLCQDTGIVGSGEGYGIIYKNNTGIGNVVIERTDNPNTVPYIPAGGGNNQMKRPPMKATLDFNAGAFNVVVYNAHTSPSGGVPKEIKLLSDRISPAHNPLDKNAVITLGDLNADGKRRLPNGQLDHGTDYYRGGFAAHPNDFPVSGWQQTVTDTDVTNFAQTLRAYDRMILSNATHNSYDEYGIIGGIPNPLPYPGGVYPAGTLFKNVHTGGGNTGKALSDHRLIWSKL

SEQ ID NO: 3 - protein, 947 aa, Unknown
  Spongosorites species
KLGDQRQIDIASWNTFDFGGKFGGVCKANDPTILQAMTDVISQHDVILVQEIANVTNPCNPANIFSGFNNLCSNNYLGGLGYLCQDTGIVGSGEGYGIIYKNNTGIGNVVIERTDNPNTVPYIPAGGGNNQMKRPPMKATLDFNAGAFNVVVYNAHTSPSGGVPKEIKLLSDRISPAHNPLDKNAVITLGDLNADGKRRLPNGQLDHGTDYYRGGFAAHPNDFPVSGWQQTVTDTDVTNFAQTLRAYDRMILSNATHNSYDEYGIIGGIPNPLPYPGGVYPAGTLFKNVHTGGGNTGKALSDHRLIWSKLNYGAIETTDNNGNTKSNFVEFTNPPQCTAPDTVYAKGADFVPNRTVDIYITKQPDNQNFIPLPQPQNPTNPGQFKQVGESYQLTDVRTTGKSTHNIDANGNIANTAVWVQPTPNQQYNLIVDVDRDGYFTNSIDIVDAFNTQGFDVDPCLRKRLAASLSDGIPAGDGTRDFVKPPKIFISGDWFPQGSARIYIIIYDKNFDYNGNDSKVLTDVRGTYDTITVGENHKFTGLEWSSPHVGNYNIIFDANSDGVYKKSDGDIVDHINEIGFAMLESYPHHSHIVDLDDNANQREDFNSGIAKNVYVLATNLPNPSTIVDVYVISEKLLKLNNPGWTTWQQSSNVPLKESAAPTNNGDRLHPTWVTPQKTVFGSVWHMPGDTFDPPYINYYGKKFTIVIDVNRDGKYDPSIDMVDTHDIGDMTDWFITQGHHDLSPTSGNGQPAVSEYKEYLNSKLDLENPLDNTNNYDAATEAASAQYLCRTNLPINLFENVIELGAQTGFIMFNPDEYYAGRDLNSGRHIYDAVDFDGLEINDGYTSVISAKEMTLNRLGVGQNSNLLTCGADTVTIDGMSTQKDSTTNIVAKTISHKSNISLEADTCTHYTHNLYFESDTNILVKAIESPFAWIIDVGDRILPGTGC

SEQ ID NO: 4 - DNA, 1911 bp, Unknown
  Spongosorites species
aactatggtgcaatagagactaccgataataacggcaatacaaagtccaattttgttgaatttacaaacccaccacaatgtactgcaccagataccgtatatgcaaagggtgcagactttgtaccaaatagaacagttgatatttacatcacaaagcagccagacaatcaaaactttatcccactgccacagccacaaaacccaacaaaccctgggcagttcaagcaagttggcgaatcataccagctaacagatgtaaggactacaggcaaaagtacgcacaacattgatgcaaacggcaacattgcaaatactgctgtatgggtacagccaacaccaaaccagcagtacaacctcatagttgatgtggaccgtgatggatactttacaaacagcatagatatagttgatgcatttaacacacaaggctttgatgtagacccatgccttcgaaagagactagcagcatcactttctgatggaataccagcaggcgatggcacacgtgactttgtaaaacctcccaaaatatttatttccggtgactggttcccacagggttctgcacgtatctacattatcatatatgacaagaactttgattataacggcaatgactccaaagtattaactgacgtccgtggaacttatgacacaattacagtgggtgagaaccacaagtttacaggactagaatggtcctcaccacatgttggtaactataacataatatttgatgcaaactctgatggagtatacaaaaaatccgatggtgacattgtagatcacataaacgagataggctttgcaatgctcgaatcgtatccacatcacagccatattgtagatttagatgacaatgcaaaccagcgagaagactttaacagtggaattgcaaaaaacgtctacgttcttgcaactaatcttccaaacccaagcacaatagttgacgtttatgtaattagtgagaaactcttaaagctaaacaatcctggatggaccacatggcagcaatcctctaatgtgccactaaaggagagtgcagcaccaactaacaatggagacaggctacaccctacttgggtaactccacaaaagacagtatttggttcagtatggcatatgcccggtgatacatttgatccaccatacattaattattacggcaaaaaattcactatagtaattgatgtgaatcgtgatggtaagtatgatcctagtatagatatggtagatactcacgatataggagacatgacagactggtttatcacacagggtcaccatgacctaagcccaactagtggaaacggacagcctgccgtatctgaatacaaagagtatctcaactcaaagctagacttggagaacccgcttgataacaccaacaactatgatgctgcaactgaggcagcttctgcacaatatctatgccgtaccaatcttccaatcaatctatttgagaatgtcatagagctgggagctcagacaggctttatcatgtttaaccctgatgagtattatgcaggccgtgatctaaattctggacgccacatttatgatgctgtagattttgatggcttggagataaatgacggatacacatcagtaatctctgcaaaagagatgaccctaaacagattaggtgttggccaaaactccaatctgcttacctgcggagctgacacagtaacgatagatggcatgtcaacacaaaaagattcaactacaaatattgttgcaaagaccatatctcacaaaagtaacatctcactagaggcagacacttgtacacattatactcacaatctctactttgagtcagacaccaacatcttagttaaggccattgagagtccatttgcatggataattgacgtaggtgacagaatacttccaggtactggctgt

SEQ ID NO: 5 - protein, 1911 aa, Artificial
  Synthetic
AATTACGGGGCCATTGAAACTACGGACAACAACGGCAACACTAAAAGCAATTTTGTCGAGTTCACCAACCCACCTCAGTGCACTGCACCAGACACGGTTTATGCTAAAGGCGCCGACTTTGTGCCCAATCGTACAGTGGATATCTATATTACTAAGCAGCCCGACAACCAAAATTTCATTCCCCTGCCGCAACCCCAAAATCCTACGAATCCTGGGCAATTCAAACAAGTTGGGGAATCATACCAATTGACGGACGTCCGTACCACCGGCAAGTCCACCCACAATATCGACGCTAACGGCAACATTGCTAACACGGCTGTATGGGTGCAGCCAACACCAAACCAACAGTACAATCTGATTGTGGATGTGGATCGCGACGGCTACTTCACGAACTCGATCGACATCGTAGATGCATTCAATACGCAGGGATTTGATGTCGATCCATGTTTGCGCAAACGTCTGGCAGCGTCACTGTCCGACGGCATCCCCGCAGGCGATGGCACGCGTGACTTTGTCAAACCTCCAAAGATTTTCATCTCAGGCGATTGGTTTCCACAGGGCAGTGCCCGCATCTACATCATTATCTATGACAAGAATTTCGATTATAACGGAAACGACAGTAAGGTTCTGACTGATGTTCGTGGCACTTATGACACAATCACGGTAGGAGAAAACCATAAGTTTACCGGCTTAGAATGGAGTTCTCCCCACGTTGGCAACTATAATATTATCTTTGATGCTAATAGCGATGGCGTTTACAAGAAATCAGATGGTGATATTGTAGACCACATTAACGAGATCGGGTTCGCCATGTTGGAGTCTTATCCACACCACTCGCACATTGTTGACTTGGATGACAATGCGAATCAACGTGAAGATTTCAACAGCGGCATTGCTAAGAACGTATACGTCTTAGCCACGAACTTACCGAACCCCTCCACAATCGTAGACGTTTACGTGATTTCAGAAAAGTTGTTGAAACTCAATAATCCTGGTTGGACCACCTGGCAACAATCCTCAAACGTGCCACTTAAGGAGAGTGCGGCACCGACAAACAATGGTGACCGTCTGCATCCAACGTGGGTAACTCCGCAGAAGACGGTATTTGGTAGCGTCTGGCACATGCCGGGCGATACATTCGACCCGCCTTACATCAATTATTATGGAAAGAAGTTTACAATCGTCATTGACGTTAACCGCGACGGCAAGTACGACCCATCAATTGATATGGTAGATACTCATGACATTGGTGACATGACTGACTGGTTCATTACGCAAGGGCACCACGATCTGTCACCGACTTCGGGCAACGGTCAACCGGCCGTGTCTGAATACAAAGAGTACCTGAATAGTAAACTCGACCTGGAAAATCCATTAGATAACACCAATAATTATGATGCAGCCACCGAAGCAGCTTCTGCCCAATATTTATGTCGTACCAACCTTCCGATCAATCTGTTCGAGAATGTAATTGAGCTTGGCGCGCAGACCGGCTTCATTATGTTCAACCCCGATGAGTATTATGCCGGGCGTGATCTTAATAGTGGCCGCCATATCTATGATGCAGTGGACTTCGATGGATTGGAGATTAATGACGGATATACGTCTGTGATCAGTGCAAAAGAAATGACGTTAAACCGCCTCGGTGTTGGCCAAAATAGTAACCTTTTGACTTGCGGCGCAGACACCGTGACAATCGATGGAATGTCTACTCAAAAGGATTCTACGACAAACATTGTAGCAAAGACAATCTCTCACAAAAGTAACATTTCGCTGGAGGCGGATACGTGCACCCACTATACGCATAACTTATATTTTGAATCCGATACGAATATCTTGGTTAAGGCAATTGAGTCCCCATTCGCTTGGATTATTGACGTGGGTGATCGTATTCTGCCCGGCACTGGCTGT

SEQ ID NO: 6 - DNA, 930 bp, Unknown
  Spongosorites species
aagcttggagatcagcgacagatagatattgcaagctggaacacatttgattttggtggcaagtttggcggtgtatgtaaggcaaatgatcctaccatactgcaagcaatgactgatgtaatatctcagcatgatgtcattttggttcaagagattgcaaatgtcacaaatccatgtaatcctgcaaacatcttttcaggctttaataatttatgttcaaataactatcttggcggtctgggatatctctgccaagatactggcattgtaggctctggtgagggctatggtattatctacaaaaacaacaccggtattggtaatgtagtaattgagcgtacagataacccaaatacagttccatacattcctgcaggtggtggcaacaaccaaatgaagaggccaccaatgaaggcaacccttgactttaatgcaggtgcattcaacgtagtagtatataatgcacacacaagcccaagtggcggtgttcccaaagagataaagctactcagtgatagaatatcgcctgctcacaatccgctagataaaaacgcagtaattacactaggtgatcttaatgccgacggcaaaagaagactgcctaatggacagctagaccatggaacagactattacagaggtggctttgcagctcacccaaatgactttcctgttagtggatggcagcagacagttactgatactgatgttacaaactttgcacagacacttcgtgcatatgatagaatgatacttagcaatgcaacacataactcttatgatgagtatggaataataggtggcattccaaacccacttccatatccgggaggagtttatcctgctggaactttgtttaaaaatgtacatactggcggtggaaatacaggaaaggcactctctgatcatagactgatatggtcaaagctt

SEQ ID NO: 7 - DNA, 930 bp, Artificial
  Synthetic
aagctcggcgatcaacgtcaaatcgatatcgcgagctggaatacgtttgacttcggtgggaaattcggcggcgtgtgcaaagccaatgacccgacgattctccaagcgatgactgatgtgatctcccagcacgatgtaattctggtccaggagatcgccaacgtgacgaacccgtgtaaccccgctaatattttcagtggcttcaataacttgtgtagtaacaactacttaggtggtttaggttatctttgccaagacactgggatcgttggtagcggtgagggctatgggatcatctacaagaacaataccggtatcggtaacgtcgtcatcgagcgcactgataacccgaatactgtgccatatattcccgccggaggcggcaacaaccagatgaagcgcccgccgatgaaggcaacgcttgatttcaatgcgggcgccttcaatgtagtcgtgtataatgcccacacgagcccatcgggcggcgtaccaaaggaaatcaaattgctctccgaccgtatcagcccagcacataatcctcttgacaagaacgcagtaattactttgggagacttgaatgcggatggaaagcgtcgcctgccaaatggacagttagatcacggtacagattattaccgcggcggatttgccgcacatccgaacgactttccggtaagtgggtggcagcaaactgtgacggatactgatgtaaccaacttcgcacaaactctccgtgcgtacgaccgcatgatcttatcaaacgccacgcacaatagttacgacgagtatgggatcatcggcggcatccctaaccctctcccctacccgggtggcgtttaccccgccggaacgttgttcaagaacgtgcatacaggcggtggcaacacaggaaaggctctcagtgatcaccgtctgatctggtctaagtta

SEQ ID NO: 8 - DNA, 2844 bp, Unknown
  Spongosorites species
aagcttggagatcagcgacagatagatattgcaagctggaacacatttgattttggtggcaagtttggcggtgtatgtaaggcaaatgatcctaccatactgcaagcaatgactgatgtaatatctcagcatgatgtcattttggttcaagagattgcaaatgtcacaaatccatgtaatcctgcaaacatcttttcaggctttaataatttatgttcaaataactatcttggcggtctgggatatctctgccaagatactggcattgtaggctctggtgagggctatggtattatctacaaaaacaacaccggtattggtaatgtagtaattgagcgtacagataacccaaatacagttccatacattcctgcaggtggtggcaacaaccaaatgaagaggccaccaatgaaggcaacccttgactttaatgcaggtgcattcaacgtagtagtatataatgcacacacaagcccaagtggcggtgttcccaaagagataaagctactcagtgatagaatatcgcctgctcacaatccgctagataaaaacgcagtaattacactaggtgatcttaatgccgacggcaaaagaagactgcctaatggacagctagaccatggaacagactattacagaggtggctttgcagctcacccaaatgactttcctgttagtggatggcagcagacagttactgatactgatgttacaaactttgcacagacacttcgtgcatatgatagaatgatacttagcaatgcaacacataactcttatgatgagtatggaataataggtggcattccaaacccacttccatatccgggaggagtttatcctgctggaactttgtttaaaaatgtacatactggcggtggaaatacaggaaaggcactctctgatcatagactgatatggtcaaagcttaactatggtgcaatagagactaccgataataacggcaatacaaagtccaattttgttgaatttacaaacccaccacaatgtactgcaccagataccgtatatgcaaagggtgcagactttgtaccaaatagaacagttgatatttacatcacaaagcagccagacaatcaaaactttatcccactgccacagccacaaaacccaacaaaccctgggcagttcaagcaagttggcgaatcataccagctaacagatgtaaggactacaggcaaaagtacgcacaacattgatgcaaacggcaacattgcaaatactgctgtatgggtacagccaacaccaaaccagcagtacaacctcatagttgatgtggaccgtgatggatactttacaaacagcatagatatagttgatgcatttaacacacaaggctttgatgtagacccatgccttcgaaagagactagcagcatcactttctgatggaataccagcaggcgatggcacacgtgactttgtaaaacctcccaaaatatttatttccggtgactggttcccacagggttctgcacgtatctacattatcatatatgacaagaactttgattataacggcaatgactccaaagtattaactgacgtccgtggaacttatgacacaattacagtgggtgagaaccacaagtttacaggactagaatggtcctcaccacatgttggtaactataacataatatttgatgcaaactctgatggagtatacaaaaaatccgatggtgacattgtagatcacataaacgagataggctttgcaatgctcgaatcgtatccacatcacagccatattgtagatttagatgacaatgcaaaccagcgagaagactttaacagtggaattgcaaaaaacgtctacgttcttgcaactaatcttccaaacccaagcacaatagttgacgtttatgtaattagtgagaaactcttaaagctaaacaatcctggatggaccacatggcagcaatcctctaatgtgccactaaaggagagtgcagcaccaactaacaatggagacaggctacaccctacttgggtaactccacaaaagacagtatttggttcagtatggcatatgcccggtgatacatttgatccaccatacattaattattacggcaaaaaattcactatagtaattgatgtgaatcgtgatggtaagtatgatcctagtatagatatggtagatactcacgatataggagacatgacagactggtttatcacacagggtcaccatgacctaagcccaactagtggaaacggacagcctgccgtatctgaatacaaagagtatctcaactcaaagctagacttggagaacccgcttgataacaccaacaactatgatgctgcaactgaggcagcttctgcacaatatctatgccgtaccaatcttccaatcaatctatttgagaatgtcatagagctgggagctcagacaggctttatcatgtttaaccctgatgagtattatgcaggccgtgatctaaattctggacgccacatttatgatgctgtagattttgatggcttggagataaatgacggatacacatcagtaatctctgcaaaagagatgaccctaaacagattaggtgttggccaaaactccaatctgcttacctgcggagctgacacagtaacgatagatggcatgtcaacacaaaaagattcaactacaaatattgttgcaaagaccatatctcacaaaagtaacatctcactagaggcagacacttgtacacattatactcacaatctctactttgagtcagacaccaacatcttagttaaggccattgagagtccatttgcatggataattgacgtaggtgacagaatacttccaggtactggctgttag

SEQ ID NO: 9 - DNA, 2848 bp, Artificial
  Synthetic
aagctcggcgatcaacgtcaaatcgatatcgcgagctggaatacgtttgacttcggtgggaaattcggcggcgtgtgcaaagccaatgacccgacgattctccaagcgatgactgatgtgatctcccagcacgatgtaattctggtccaggagatcgccaacgtgacgaacccgtgtaaccccgctaatattttcagtggcttcaataacttgtgtagtaacaactacttaggtggtttaggttatctttgccaagacactgggatcgttggtagcggtgagggctatgggatcatctacaagaacaataccggtatcggtaacgtcgtcatcgagcgcactgataacccgaatactgtgccatatattcccgccggaggcggcaacaaccagatgaagcgcccgccgatgaaggcaacgcttgatttcaatgcgggcgccttcaatgtagtcgtgtataatgcccacacgagcccatcgggcggcgtaccaaaggaaatcaaattgctctccgaccgtatcagcccagcacataatcctcttgacaagaacgcagtaattactttgggagacttgaatgcggatggaaagcgtcgcctgccaaatggacagttagatcacggtacagattattaccgcggcggatttgccgcacatccgaacgactttccggtaagtgggtggcagcaaactgtgacggatactgatgtaaccaacttcgcacaaactctccgtgcgtacgaccgcatgatcttatcaaacgccacgcacaatagttacgacgagtatgggatcatcggcggcatccctaaccctctcccctacccgggtggcgtttaccccgccggaacgttgttcaagaacgtgcatacaggcggtggcaacacaggaaaggctctcagtgatcaccgtctgatctggtctaagttaaattacggggccattgaaactacggacaacaacggcaacactaaaagcaattttgtcgagttcaccaacccacctcagtgcactgcaccagacacggtttatgctaaaggcgccgactttgtgcccaatcgtacagtggatatctatattactaagcagcccgacaaccaaaatttcattcccctgccgcaaccccaaaatcctacgaatcctgggcaattcaaacaagttggggaatcataccaattgacggacgtccgtaccaccggcaagtccacccacaatatcgacgctaacggcaacattgctaacacggctgtatgggtgcagccaacaccaaaccaacagtacaatctgattgtggatgtggatcgcgacggctacttcacgaactcgatcgacatcgtagatgcattcaatacgcagggatttgatgtcgatccatgtttgcgcaaacgtctggcagcgtcactgtccgacggcatccccgcaggcgatggcacgcgtgactttgtcaaacctccaaagattttcatctcaggcgattggtttccacagggcagtgcccgcatctacatcattatctatgacaagaatttcgattataacggaaacgacagtaaggttctgactgatgttcgtggcacttatgacacaatcacggtaggagaaaaccataagtttaccggcttagaatggagttctccccacgttggcaactataatattatctttgatgctaatagcgatggcgtttacaagaaatcagatggtgatattgtagaccacattaacgagatcgggttcgccatgttggagtcttatccacaccactcgcacattgttgacttggatgacaatgcgaatcaacgtgaagatttcaacagcggcattgctaagaacgtatacgtcttagccacgaacttaccgaacccctccacaatcgtagacgtttacgtgatttcagaaaagttgttgaaactcaataatcctggttggaccacctggcaacaatcctcaaacgtgccacttaaggagagtgcggcaccgacaaacaatggtgaccgtctgcatccaacgtgggtaactccgcagaagacggtatttggtagcgtctggcacatgccgggcgatacattcgacccgccttacatcaattattatggaaagaagtttacaatcgtcattgacgttaaccgcgacggcaagtacgacccatcaattgatatggtagatactcatgacattggtgacatgactgactggttcattacgcaagggcaccacgatctgtcaccgacttcgggcaacggtcaaccggccgtgtctgaatacaaagagtacctgaatagtaaactcgacctggaaaatccattagataacaccaataattatgatgcagccaccgaagcagcttctgcccaatatttatgtcgtaccaaccttccgatcaatctgttcgagaatgtaattgagcttggcgcgcagaccggcttcattatgttcaaccccgatgagtattatgccgggcgtgatcttaatagtggccgccatatctatgatgcagtggacttcgatggattggagattaatgacggatatacgtctgtgatcagtgcaaaagaaatgacgttaaaccgcctcggtgttggccaaaatagtaaccttttgacttgcggcgcagacaccgtgacaatcgatggaatgtctactcaaaaggattctacgacaaacattgtagcaaagacaatctctcacaaaagtaacatttcgctggaggcggatacgtgcacccactatacgcataacttatattttgaatccgatacgaatatcttggttaaggcaattgagtccccattcgcttggattattgacgtgggtgatcgtattctgcccggcactggctgtctcgaga

SEQ ID NO: 10 - protein, 24 aa, Unknown
  Spongosorites species
  misc_feature (21)..(21): Unknown - any amino acid
KLGDQRQIDIASWNTFDGGVXKAN

SEQ ID NO: 11 - protein, 3 aa, Unknown
  Spongosorites species
  misc_feature (1)..(1): Unknown - any amino acid
XGH

SEQ ID NO: 12 - protein, 7 aa, Unknown
  Spongosorites species
  misc_feature (4)..(6): Unknown - any amino acid
TGAXXXE

SEQ ID NO: 13 - protein, 5 aa, Unknown
  Spongosorites species
  misc_feature (3)..(3): Unknown - any amino acid
SGXST

SEQ ID NO: 14 - protein, 13 aa, Unknown
  Spongosorites species
  misc_feature (1)..(1): Q or G
  misc_feature (8)..(8): Unknown - any amino acid
  misc_feature (10)..(10): Unknown - any amino acid
  misc_feature (12)..(12): Unknown - any amino acid
XAGFVPNXTXDXT

SEQ ID NO: 15 - protein, 4 aa, Unknown
  Spongosorites species
NFDY

SEQ ID NO: 16 - protein, 13 aa, Unknown
  Spongosorites species
  misc_feature (12)..(12): Unknown - any amino acid
LALEVPLRTVNXT

SEQ ID NO: 17 - protein, 14 aa, Unknown
  Spongosorites species
  misc_feature (6)..(6): Unknown - any amino acid
  misc_feature (10)..(10): Unknown - any amino acid
  misc_feature (12)..(12): Unknown - any amino acid
GDGENXNDNXDXYD

SEQ ID NO: 18 - protein, 13 aa, Unknown
  Spongosorites species
  misc_feature (9)..(9): Unknown - any amino acid
  misc_feature (11)..(12): Unknown - any amino acid
ASTGSTIPXGXXT

SEQ ID NO: 19 - protein, 9 aa, Unknown
  Spongosorites species
  misc_feature (9)..(9): E or G
ESAAETENX

SEQ ID NO: 20 - protein, 19 aa, Unknown
  Spongosorites species
  misc_feature (9)..(9): N or A
  misc_feature (10)..(10): Unknown - any amino acid
  misc_feature (17)..(17): Unknown - any amino acid
  misc_feature (19)..(19): E or G
ASAAPTNNXXTSLSSGXDX

SEQ ID NO: 21 - protein, 8 aa, Unknown
  Spongosorites species
  misc_feature (1)..(1): Unknown - any amino acid
  misc_feature (4)..(4): D or E
XLDXETLE

SEQ ID NO: 22 - protein, 13 aa, Unknown
  Spongosorites species
  misc_feature (9)..(9): D or E
  misc_feature (10)..(12): Unknown - any amino acid
PLDVRGTYXXXXV

SEQ ID NO: 23 - protein, 14 aa, Unknown
  Spongosorites species
  misc_feature (2)..(2): Unknown - any amino acid
  misc_feature (10)..(10): Unknown - any amino acid
  misc_feature (13)..(13): Unknown - any amino acid
HXQDRIQPAXPPXH

SEQ ID NO: 24 - protein, 33 aa, Unknown
  Spongosorites species
  misc_feature (25)..(25): A or G
  misc_feature (27)..(27): Unknown - any amino acid
  misc_feature (32)..(32): Unknown - any amino acid
ANTGIGNVVIERTDNPNTVPYIPAXGXNNNHXH

SEQ ID NO: 25 - protein, 15 aa, Unknown
  Spongosorites species
  misc_feature (14)..(14): Unknown - any amino acid
FAVITLGDLNADGXH

SEQ ID NO: 26 - protein, 3 aa, Unknown
  Spongosorites species
LPG

SEQ ID NO: 27 - DNA, 21 bp, Artificial
  Synthetic
  misc_feature (6)..(6): n is a, c, g, or t
  misc_feature (21)..(21): n is a, c, g, or t
carmgncarathgayathgcn

SEQ ID NO: 28 - DNA, 20 bp, Artificial
  Synthetic
  misc_feature (3)..(3): n is a, c, g, or t
  misc_feature (6)..(6): n is a, c, g, or t
  misc_feature (9)..(9): n is a, c, g, or t
  misc_feature (12)..(12): n is a, c, g, or t
  misc_feature (15)..(15): n is a, c, g, or t
  misc_feature (18)..(18): n is a, c, g, or t
atngynswnccngtnswngc

SEQ ID NO: 29 - DNA, 20 bp, Artificial
  Synthetic
  misc_feature (9)..(9): n is a, c, g, or t
tggaayacnttygayttygg

SEQ ID NO: 30 - DNA, 20 bp, Artificial
  Synthetic
  misc_feature (3)..(3): n is a, c, g, or t
  misc_feature (6)..(6): n is a, c, g, or t
  misc_feature (9)..(9): n is a, c, g, or t
  misc_feature (18)..(18): n is a, c, g, or t
ccngcngcnggdatrtangg

SEQ ID NO: 31 - DNA, 28 bp, Artificial
  Synthetic
caaatgtcacaaatccatgtaatcctgc

SEQ ID NO: 32 - DNA, 22 bp, Artificial
  Synthetic
atgatcctaccatactgcaagc

SEQ ID NO: 33 - DNA, 23 bp, Artificial
  Synthetic
gatactggcattgtaggctctgg

SEQ ID NO: 34 - DNA, 22 bp, Artificial
  Synthetic
ggcactctctgatcatagactg

SEQ ID NO: 35 - DNA, 25 bp, Artificial
  Synthetic
cagctgatgaggtatacgtcaaagg

SEQ ID NO: 36 - DNA, 28 bp, Artificial
  Synthetic
aatctcttgaaccaaaatgacatcatgc

SEQ ID NO: 37 - DNA, 23 bp, Artificial
  Synthetic
cttacatacaccgccaaacttgc

SEQ ID NO: 38 - DNA, 28 bp, Artificial
  Synthetic
gtgctggttttagatgtgtggcttgtgc

SEQ ID NO: 39 - DNA, 28 bp, Artificial
  Synthetic
tctggacatatcaaggccctgcaggtgg

SEQ ID NO: 40 - DNA, 28 bp, Artificial
  Synthetic
ccattaaacttgaaaacatgacagggcc

SEQ ID NO: 41 - DNA, 28 bp, Artificial
  Synthetic
tgatcttacagtacaaccagcagcactg

SEQ ID NO: 42 - DNA, 45 bp, Artificial
  Synthetic
ctaatacgactcactatagggcaagcagtggtatcaacgcagagt

SEQ ID NO: 43 - DNA, 22 bp, Artificial
  Synthetic
ctaatacgactcactatagggc

SEQ ID NO: 44 - DNA, 23 bp, Artificial
  Synthetic
aagcagtggtatcaacgcagagt

SEQ ID NO: 45 - DNA, 27 bp, Artificial
  Synthetic
gtcgacaagcttggagatcagcgacag

SEQ ID NO: 46 - DNA, 27 bp, Artificial
  Synthetic
gaattcctaacagccagtacctggaag

SEQ ID NO: 47 - DNA, 344 bp, Artificial
  Synthetic
tggaatacttttgactttggtggcaagtttggcggtgtatgtaaggcaaatgatcctaccatactgcaagcaatgactgatgtaatatctcagcatgatgtcattttggttcaagagattgcaaatgtcacaaatccatgtaatcctgcaaacatcttttcaggctttaataatttatgttcaaataactatcttggcggtctgggatatctctgccaagatactggcattgtaggctctggtgagggctatggtattatctacaaaaacaacaccggtattggtaatgtagtaattgagcgtacagataacccaaatacagttccatatataccagcagcagg

SEQ ID NO: 48 - protein, 115 aa, Artificial
  Synthetic
WNTFDFGGKFGGVCKANDPTILQAMTDVISQHDVILVQEIANVTNPCNPANIFSGFNNLCSNNYLGGLGYLCQDTGIVGSGEGYGIIYKNNTGIGNVVIERTDNPNTVPYIPAAG

SEQ ID NO: 49 - DNA, 864 bp, Artificial
  Synthetic
gatactggcattgtaggctctggtgagggctatggtattatctacaaaaacaacaccggtattggtaatgtagtaattgagcgtacagataacccaaatacagttccatacattcctgcaggtggtggcaacaaccaaatgaagaggccaccaatgaaggcaacccttgactttaatgcaggtgcattcaacgtagtagtatataatgcacacacaagcccaagtggcggtgttcccaaagagataaagctactcagtgatagaatatcgcctgctcacaatccgctagataaaaacgcagtaattacactaggtgatcttaatgccgacggcaaaagaagactgcctaatggacagctagaccatggaacagactattacagaggtggctttgcagctcacccaaatgactttcctgttagtggatggcagcagacagttactgatactgatgttacaaactttgcacagacacttcgtgcatatgatagaatgatacttagcaatgcaacacataactcttatgatgagtatggaataataggtggcattccaaacccacttccatatccgggaggagtttatcctgctggaactttgtttaaaaatgtacatactggcggtggaaatacaggaaaggcactctctgatcatagactgatatggtcaaagcttaactatggtgcaatagagactaccgataataacggcaatacaaagtccaattttgttgaatttacaaacccaccacaatgtactgcaccagataccgtatatgcaaagggtgcagactttgtaccaaatagaacagttgatatttacatcacaaagcagccagacaatcaaaactttatcccactgcca

SEQ ID NO: 50 - protein, 295 aa, Artificial
  Synthetic
DTGIVGSGEGYGIIYKNNTGIGNVVIERTDNPNTVPYIPAGGGNNQMKRPPMKATLDFNAGAFNVVVYNAHTSPSGGVPKEIKLLSDRISPAHNPLDKNAVITLGDLNADGKRRLPNGQLDHGTDYYRGGFAAHPNDFPVSGWQQTVTDTDVTNFAQTLRAYDRMILSNATHNSYDEYGIIGGIPNPLPYPGGVYPAGTLFKNVHTGGGNTGKALSDHRLIWSKLNYGAIETTDNNGNTKSNFVEFTNPPQCTAPDTVYAKGADFVPNRTVDIYITKQPDNQNFIPLPEPTMPVS

SEQ ID NO: 51 - DNA, 2511 bp, Artificial
  Synthetic
ggcactctctgatcatagactgatatggtcaaagcttaactatggtgcaatagagactaccgataataacggcaatacaaagtccaattttgttgaatttacaaacccaccacaatgtactgcaccagataccgtatatgcaaagggtgcagactttgtaccaaatagaacagttgatatttacatcacaaagcagccagacaatcaaaactttatcccactgccacagccacaaaacccaacaaaccctgggcagttcaagcaagttggcgaatcataccagctaacagatgtaaggactacaggcaaaagtacgcacaacattgatgcaaacggcaacattgcaaatactgctgtatgggtacagccaacaccaaaccagcagtactacctcatagttgatgtggaccgtgatggatactttacaaacagcatagatatagttgatgcatttaacacacaaggctttgatgtagacccatgccttcgaaagagactagcagcatcactttctgatggaataccagcaggcgatggcacacgtgactttgtaaaacctcccaaaatatttatttccggtgactggttcccacagggttctgcacgtatctacattatcatatatgacaagaactttgattataacggcaatgactccaaagtattaactgacgtccgtggaacttatgacacaattacagtgggtgagaaccacaagtttacaggactagaatggtcctcaccacatgttggtaactataacataatatttgatgcaaactctgatggagtatacaaaaaatccgatggtgacattgtagatcacataaacgagataggctttgcaatgctcgaatcgtatccacatcacagccatattgtagatttagatgacaatgcaaaccagcgagaagactttaacagtggaattgcaaaaaacgtctacgttcttgcaactaatcttccaaacccaagcacaatagttgacgtttatgtaattagtgagaaactcttaaagctaaacaatcctggatggaccacatggcagcaatcctctaatgtgctactaaaggagagtgcagcaccaactaacaatggagacaggctacaccctacttgggtaactccacaaaagacagtatttggttcagtatggcatatgcccggtgatacatttgatccaccatacattaattattacggcaaaaaattcactatagtaattgatgtgaatcgtgatggtaagtatgatcctagtatagatatggtagatactcacgatataggagacatgacagactggtttatcacacagggtcaccatgacctaagcccaactagtggaaacggacagcctgccgtatctgaatacaaagagtatctcaactcaaagctagacttggagaacccgcttgataacaccaacaactatgatgctgcaactgaggcagcttctgcacaatatctatgccgtaccaatcttccaatcaatctatttgagaatgtcatagagctgggagctcagacaggctttatcatgtttaaccctgatgagtattatgcaggccgtgatctaaattctggacgccacatttatgatgctgtagattttgatggcttggagataaatgacggatacacatcagtaatctctgcaaaagagatgaccctaaacagattaggtgttggccaaaactccaatctgcttacctgcggagctgacacagtaacgatagatggcatgtcaacacaaaaagattcaactacaaatattgttgcaaagaccatatctcacaaaagtaacatctcactagaggcagacacttgtacacattatactcacaatctctactttgagtcagacaccaacatcttagttaaggccattgagagtccatttgcatggataattgacgtaggtgacagaatacttccaggtactggctgttagatgaatcgtgtgtgtgggttcttctaatatgattgcacaagccacacatctaaaaccagcacaatgcacacacaccttctgctgttttataactttaatacattatgacattttactctaaattgcaataatagttaatcagtgtatatactcaaaaacaaaaaactatccctcataccattgagtgctatgatattatttttgggaataataataataataatgggggggggcatcttcgcagtccttttctcaaactgatatatagataaactaaatgacaattcacaagatctgggtaatctatagattacttaacatgatgttatcatgctatcctttctatgggggccacctgcagggccttgatatgtccagataaaaaacaggtcaaattacgtcccaccttttgtacagatctcctactatcgaatatctaaaataacggctaaaatagccaaacgctaggaaggttatctaatgagcttaaaaaaaaaacaagaaacaaaaaaaaaaaaaaaaaaaaaaaaaaaaagtactctgcgttgataccactgctt

SEQ ID NO: 52 - protein, 649 aa, Artificial
  Synthetic
ALSDHRLIWSKLNYGAIETTDNNGNTKSNFVEFTNPPQCTAPDTVYAKGADFVPNRTVDIYITKQPDNQNFIPLPQPQNPTNPGQFKQVGESYQLTDVRTTGKSTHNIDANGNIANTAVWVQPTPNQQYYLIVDVDRDGYFTNSIDIVDAFNTQGFDVDPCLRKRLAASLSDGIPAGDGTRDFVKPPKIFISGDWFPQGSARIYIIIYDKNFDYNGNDSKVLTDVRGTYDTITVGENHKFTGLEWSSPHVGNYNIIFDANSDGVYKKSDGDIVDHINEIGFAMLESYPHHSHIVDLDDNANQREDFNSGIAKNVYVLATNLPNPSTIVDVYVISEKLLKLNNPGWTTWQQSSNVLLKESAAPTNNGDRLHPTWVTPQKTVFGSVWHMPGDTFDPPYINYYGKKFTIVIDVNRDGKYDPSIDMVDTHDIGDMTDWFITQGHHDLSPTSGNGQPAVSEYKEYLNSKLDLENPLDNTNNYDAATEAASAQYLCRTNLPINLFENVIELGAQTGFIMFNPDEYYAGRDLNSGRHIYDAVDFDGLEINDGYTSVISAKEMTLNRLGVGQNSNLLTCGADTVTIDGMSTQKDSTTNIVAKTISHKSNISLEADTCTHYTHNLYFESDTNILVKAIESPFAWIIDVGDRILPGTGC

SEQ ID NO: 53 - DNA, 1607 bp, Artificial
  Synthetic
gatactggcattgtaggctctggtgagggctatggtattatctacaaaaacaacaccggtattggtaatgtagtaattgagcgtacagataacccaaatacagttccatacaggcaatgactatagatagtacaggcaaggtaggaattggaactactagtccaaatgcaaaattacatgtacaaggatcatttagagtccaaggctataacatcttaactgaccctgatgaacaatacaacagtcattttcctcattcaagtaataatgtctacatctccggtgataaaacatatcttagaagtggcaaaccaaacggttactccactacaatgactgttgataacatcaatggtaatgtaggcattggactaaaagagcccaaagtaaaacttcatatgtttggtgatgcctttttgcaaacaggctcttctggcgattcaagaaatttgcaattaagcagtttctatacttcacaaccaaaagctatcaatcttgagagtcgttactttgatacgatagagggattcaaacttggagcaagtggagatagttttgtccatgacaagttctacataaatagatacactgcccatgtaaatgacaatcccggaattacaactaacaacgttgattcaaccagactcgttacagttacagcaactgggaatgtaggaattggaactactagtccaagtaagacacttgatgtaaatggcgatgccattgcaaataactggcttagacattccagccagcaatggaaaacagacatagcaccactagataactccctaagcaagctagagcagctacaaggcataagctacaagtggaaagcagatgaattcgcagacatgggatttcacaataaaacaactcttgggtttattgccgaagatgtagagactatacttccacagctaatccatactgaccaaaatggtgaaaaatcaatggactatggtaaactaacaccaattctagttgaagcaatcaaagagcagcaaaacaccatagaacagctaaccactgcaatatgtaaagacagcccaaaacatgacgtgtgcagcagctaaaaaacacttttttttaattttttgatcttacagtacaaccagcagcactgaaacatttttggcatcgaatctaaaaagaaaaaaaaatcatactatcacataatgatgatatgctgtattgtgagaaactcaaagacactgaaaaacatttttggcatcgaatctaaaaagaaaaaaaaatcatactatcacataatgatgatgatgatgctgtattgtgagaaactcaaagacactgaaacattttatacaatccattaaacttgaaaacatgacagggccaacaaatctcaaaatacttcctatataccttacacgccataacacttctcattgaaacgacaagatagtagtattacacatcagactgtgtcaattttttgcattaccatcttgcttttatcagttagtgcatactttcctaattctattccagaggcgcaagctaagcttggagatcagcgacagatagatattgcaagctggaacacatttgattttggtggcaagtttggcggtgtatgtaag

SEQ ID NO: 54 - protein, 37 aa, Artificial
  Synthetic
DTGIVGSGEGYGIIYKNNTGIGNVVIERTDNPNTVPY

SEQ ID NO: 55 - protein, 68 aa, Artificial
  Synthetic
HFSLKRQDSSITHQTVSIFCITILLLSVSAYFPNSIPEAQAKLGDQRQIDIASWNTFDFGGKFGGVCK

SEQ ID NO: 56 - DNA, 3099 bp, Artificial
  Synthetic
ccattaaacttgaaaacatgacagggccaacaaatctcaaaatacttcctatataccttacacgccataacacttctcattgaaacgacaagatagtagtattacacatcagactgtgtcaattttttgcattaccatcttgcttttatcagttagtgcatactttcctaattctattccagaggcgcaagctaagcttggagatcagcgacagatagatattgcaagctggaacacatttgattttggtggcaagtttggcggtgtatgtaaggcaaatgatcctaccatactgcaagcaatgactgatgtaatatctcagcatgatgtcattttggttcaagagattgcaaatgtcacaaatccatgtaatcctgcaaacatcttttcaggctttaataatttatgttcaaataactatcttggcggtctgggatatctctgccaagatactggcattgtaggctctggtgagggctatggtattatctacaaaaacaacaccggtattggtaatgtagtaattgagcgtacagataacccaaatacagttccatacattcctgcaggtggtggcaacaaccaaatgaagaggccaccaatgaaggcaacccttgactttaatgcaggtgcattcaacgtagtagtatataatgcacacacaagcccaagtggcggtgttcccaaagagataaagctactcagtgatagaatatcgcctgctcacaatccgctagataaaaacgcagtaattacactaggtgatcttaatgccgacggcaaaagaagactgcctaatggacagctagaccatggaacagactattacagaggtggctttgcagctcacccaaatgactttcctgttagtggatggcagcagacagttactgatactgatgttacaaactttgcacagacacttcgtgcatatgatagaatgatacttagcaatgcaacacataactcttatgatgagtatggaataataggtggcattccaaacccacttccatatccgggaggagtttatcctgctggaactttgtttaaaaatgtacatactggcggtggaaatacaggaaaggcactctctgatcatagactgatatggtcaaagcttaactatggtgcaatagagactaccgataataacggcaatacaaagtccaattttgttgaatttacaaacccaccacaatgtactgcaccagataccgtatatgcaaagggtgcagactttgtaccaaatagaacagttgatatttacatcacaaagcagccagacaatcaaaactttatcccactgccacagccacaaaacccaacaaaccctgggcagttcaagcaagttggcgaatcataccagctaacagatgtaaggactacaggcaaaagtacgcacaacattgatgcaaacggcaacattgcaaatactgctgtatgggtacagccaacaccaaaccagcagtacaacctcatagttgatgtggaccgtgatggatactttacaaacagcatagatatagttgatgcatttaacacacaaggctttgatgtagacccatgccttcgaaagagactagcagcatcactttctgatggaataccagcaggcgatggcacacgtgactttgtaaaacctcccaaaatatttatttccggtgactggttcccacagggttctgcacgtatctacattatcatatatgacaagaactttgattataacggcaatgactccaaagtattaactgacgtccgtggaacttatgacacaattacagtgggtgagaaccacaagtttacaggactagaatggtcctcaccacatgttggtaactataacataatatttgatgcaaactctgatggagtatacaaaaaatccgatggtgacattgtagatcacataaacgagataggctttgcaatgctcgaatcgtatccacatcacagccatattgtagatttagatgacaatgcaaaccagcgagaagactttaacagtggaattgcaaaaaacgtctacgttcttgcaactaatcttccaaacccaagcacaatagttgacgtttatgtaattagtgagaaactcttaaagctaaacaatcctggatggaccacatggcagcaatcctctaatgtgccactaaaggagagtgcagcaccaactaacaatggagacaggctacaccctacttgggtaactccacaaaagacagtatttggttcagtatggcatatgcccggtgatacatttgatccaccatacattaattattacggcaaaaaattcactatagtaattgatgtgaatcgtgatggtaagtatgatcctagtatagatatggtagatactcacgatataggagacatgacagactggtttatcacacagggtcaccatgacctaagcccaactagtggaaacggacagcctgccgtatctgaatacaaagagtatctcaactcaaagctagacttggagaacccgcttgataacaccaacaactatgatgctgcaactgaggcagcttctgcacaatatctatgccgtaccaatcttccaatcaatctatttgagaatgtcatagagctgggagctcagacaggctttatcatgtttaaccctgatgagtattatgcaggccgtgatctaaattctggacgccacatttatgatgctgtagattttgatggcttggagataaatgacggatacacatcagtaatctctgcaaaagagatgaccctaaacagattaggtgttggccaaaactccaatctgcttacctgcggagctgacacagtaacgatagatggcatgtcaacacaaaaagattcaactacaaatattgttgcaaagaccatatctcacaaaagtaacatctcactagaggcagacacttgtacacattatactcacaatctctactttgagtcagacaccaacatcttagttaaggccattgagagtccatttgcatggataattgacgtaggtgacagaatacttccaggtactggctgttagatgaatcgtgtgtgtgggttcttctaatatgattgcacaagccacacatctaaaaccagcac

SEQ ID NO: 57 - protein, 988 aa, Artificial
  Synthetic
HFSLKRQDSSITHQTVSIFCITILLLSVSAYFPNSIPEAQAKLGDQRQIDIASWNTFDFGGKFGGVCKANDPTILQAMTDVISQHDVILVQEIANVTNPCNPANIFSGFNNLCSNNYLGGLGYLCQDTGIVGSGEGYGIIYKNNTGIGNVVIERTDNPNTVPYIPAGGGNNQMKRPPMKATLDFNAGAFNVVVYNAHTSPSGGVPKEIKLLSDRISPAHNPLDKNAVITLGDLNADGKRRLPNGQLDHGTDYYRGGFAAHPNDFPVSGWQQTVTDTDVTNFAQTLRAYDRMILSNATHNSYDEYGIIGGIPNPLPYPGGVYPAGTLFKNVHTGGGNTGKALSDHRLIWSKLNYGAIETTDNNGNTKSNFVEFTNPPQCTAPDTVYAKGADFVPNRTVDIYITKQPDNQNFIPLPQPQNPTNPGQFKQVGESYQLTDVRTTGKSTHNIDANGNIANTAVWVQPTPNQQYNLIVDVDRDGYFTNSIDIVDAFNTQGFDVDPCLRKRLAASLSDGIPAGDGTRDFVKPPKIFISGDWFPQGSARIYIIIYDKNFDYNGNDSKVLTDVRGTYDTITVGENHKFTGLEWSSPHVGNYNIIFDANSDGVYKKSDGDIVDHINEIGFAMLESYPHHSHIVDLDDNANQREDFNSGIAKNVYVLATNLPNPSTIVDVYVISEKLLKLNNPGWTTWQQSSNVPLKESAAPTNNGDRLHPTWVTPQKTVFGSVWHMPGDTFDPPYINYYGKKFTIVIDVNRDGKYDPSIDMVDTHDIGDMTDWFITQGHHDLSPTSGNGQPAVSEYKEYLNSKLDLENPLDNTNNYDAATEAASAQYLCRTNLPINLFENVIELGAQTGFIMFNPDEYYAGRDLNSGRHIYDAVDFDGLEINDGYTSVISAKEMTLNRLGVGQNSNLLTCGADTVTIDGMSTQKDSTTNIVAKTISHKSNISLEADTCTHYTHNLYFESDTNILVKAIESPFAWIIDVGDRILPGTGC

SEQ ID NO: 58 - DNA, 930 bp, Artificial
  Synthetic
aaactgggagatcagcggcaaattgacatcgcttcatggaacacctttgactttggcgggaaattcggtggtgtctgtaaagccaatgatccgacgattctccaggccatgactgatgtgatttcccagcatgacgtcatcctggtgcaggagattgccaatgtgaccaacccgtgtaatcccgccaacattttctctgggttcaataacctgtgctccaacaactacctgggcggtcttggctatttgtgccaagacacgggcatcgttggtagtggcgaaggctacgggattatctacaagaataacaccggaatcggaaatgtggtcattgaacgtaccgataatccgaatacggttccgtatattcccgctggtggcggtaacaaccagatgaaacgcccaccgatgaaagcaaccctggacttcaacgcaggcgcgtttaacgtagtggtctataacgcgcatacaagcccatcaggtggtgtaccgaaagaaatcaaactgctgtctgaccgcattagccctgcccataatccgttagacaagaacgcggtgattactctgggcgatctgaatgcggatgggaaacgtcgcttaccaaatggccaactcgatcacgggacggattactaccgtggtggttttgccgcgcatccgaacgattttccggtttcgggctggcaacagacagtgacagatacggatgttacgaactttgcgcagaccttacgtgcgtatgatcgcatgatccttagcaatgcaacccacaactcgtacgatgagtatggcattatcggcggaatcccaaacccgttgccttatccgggtggcgtttatcctgctgggaccctgttcaagaacgtacacactggcggtggcaataccggtaaagcactgagtgaccatcgcctgatttggagcaaattg

SEQ ID NO: 59 - DNA, 948 bp, Artificial
  Synthetic
gtcgacaaactgggagatcagcggcaaattgacatcgcttcatggaacacctttgactttggcgggaaattcggtggtgtctgtaaagccaatgatccgacgattctccaggccatgactgatgtgatttcccagcatgacgtcatcctggtgcaggagattgccaatgtgaccaacccgtgtaatcccgccaacattttctctgggttcaataacctgtgctccaacaactacctgggcggtcttggctatttgtgccaagacacgggcatcgttggtagtggcgaaggctacgggattatctacaagaataacaccggaatcggaaatgtggtcattgaacgtaccgataatccgaatacggttccgtatattcccgctggtggcggtaacaaccagatgaaacgcccaccgatgaaagcaaccctggacttcaacgcaggcgcgtttaacgtagtggtctataacgcgcatacaagcccatcaggtggtgtaccgaaagaaatcaaactgctgtctgaccgcattagccctgcccataatccgttagacaagaacgcggtgattactctgggcgatctgaatgcggatgggaaacgtcgcttaccaaatggccaactcgatcacgggacggattactaccgtggtggttttgccgcgcatccgaacgattttccggtttcgggctggcaacagacagtgacagatacggatgttacgaactttgcgcagaccttacgtgcgtatgatcgcatgatccttagcaatgcaacccacaactcgtacgatgagtatggcattatcggcggaatcccaaacccgttgccttatccgggtggcgtttatcctgctgggaccctgttcaagaacgtacacactggcggtggcaataccggtaaagcactgagtgaccatcgcctgatttggagcaaattgaattaaaagctt

SEQ ID NO: 60 - DNA, 2966 bp, Artificial
  Synthetic
atcagatcggaagagcgtcgttaagggaaagagtgttccgtactcagagagctattacaattcactggccgtcgttttacaacgtcgtgactgggaaaacccaggcgttacccaacttaatcgccttgcagcacatcctccgtttgcgagttggcggaatagcgaagaagcgcgtacagatcgtccgtcacagcagttgcgctctctgtaacgtaccgcagttcaaagtctacacctacaaacgcgaaagtcgctatcgcctgtttgtggatgtgcaaagcgacatcatcgatactcctggtcgtcgcacggtgattccgttggcttctgcacggttactgagcgacaaagtttcccgtgaactctatccggttgtccacattggggatgaaagctggcgtatgatgaccacggatatggcgtcagtaccagtgtcggtaattggcgaagaggttgcggatctgtcgcatcgcgagaatgacatcaagaacgccattaacctgatgttttggggcatttaattaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgaccatgattacgccaagctatctgggcgataccattgaagactggagttcagacgtgtgctcttccgatctgat

(19) United States
(12) Patent Application Publication
(10) Pub. No.: US 2023/0242598 A1
(43) Pub. Date: Aug. 3, 2023

(54) FUNCTIONAL POLYPEPTIDES AND USE THEREOF

(71) Applicant: National University Corporation Hokkaido University, Hokkaido (JP)

(72) Inventors: Ryuichi SAKAI, Hokkaido (JP); Koji NAKANO, Hokkaido (JP)

(73) Assignee: National University Corporation Hokkaido University, Hokkaido (JP)

(21) Appl. No.: 17/642,339

(22) PCT Filed: Apr. 7, 2021
(86) PCT No.: PCT/JP2021/015369

§ 371 Date: Mar. 11, 2022

(30) Foreign Application Priority Data
Apr. 7, 2020 (JP) 2020-069328

(51) Int. Cl. C07K 14/435; C12N 15/70; C12N 5/10

(52) U.S. Cl. CPC C07K 14/435; C12N 15/70; C12N 5/10

(57) ABSTRACT

A polypeptide that comprises the amino acid sequence of SEQ ID NO: 1 and has a function of delivering a substance into cells, a polypeptide that comprises the amino acid sequence of SEQ ID NO: 2 and has a function of degrading a nucleic acid molecule, a polypeptide that comprises the amino acid sequence of SEQ ID NO: 3 and has a function of degrading a nucleic acid molecule and a function of intranuclearly migrating the same, polynucleotides encoding the respective polypeptides, and transformed cells containing the respective polynucleotides.

A sequence listing in electronic (ASCII text file) format is filed with this application and incorporated herein by reference. The name of the ASCII text file is “2022_0170A_ST25.txt”; the file was created on Aug. 12, 2022; the size of the file is 93,894 bytes.

### TECHNICAL FIELD

The present disclosure relates to a polypeptide having a function of delivering a substance into a cell, a polypeptide having a function of degrading a nucleic acid molecule, a polynucleotide encoding any of these polypeptides, etc.

### BACKGROUND ART

Marine organisms are regarded as a great source of physiologically active substances, and many secondary metabolites that exhibits various physiological activities such as antiviral activity, antiproliferative activity, antioxidative activity, and anticoagulating activity have been found therefrom (Non-Patent Literature 1). In the meantime, it has been reported that sponges of the genus Spongosorites contain proteins having strong toxicity and having a function of translocating into the cell nucleus (Non-Patent Literatures 2 to 4).

### CITATION LIST

### Non-Patent Literature

Non-Patent Literature 1: Gogineni and Hamann, Biochim Biophys Acta Gen Subj. 2018; 1862(1):81-196

Non-Patent Literature 2: Yuusuke Ida et al., “Mechanism of action of novel proteinous toxin derived from sponge of the genus Spongosorites from Iriomote Island”, Abstracts for the annual meeting of the Japanese Society of Fisheries Science, 2018 Spring, 107 Non-Patent Literature 3: http://asp2017.org/wp-content/uploads/2016/12/ASP20201720Annual20Meeting_web.pdf, p. 28, S-12 Non-Patent Literature 4: http://dokuso-symposium.bio.tokushima-u.ac.jp/program/62th-abstract/62th-o.html, 0-11

### DISCLOSURE OF THE INVENTION

The exploitation of a useful physiologically active substance derived from a marine organism is expected.

Some aspects of the present disclosure provide the following.

[1] A polypeptide having a function of delivering a substance into a cell, the polypeptide being selected from
(a) a polypeptide comprising the amino acid sequence of SEQ ID NO: 1,
(b) a polypeptide that comprises an amino acid sequence containing a mutation of one or more amino acids in the amino acid sequence of SEQ ID NO: 1, and has the function of delivering a substance into a cell,
(c) a polypeptide that comprises an amino acid sequence having 80% or higher sequence identity to the amino acid sequence of SEQ ID NO: 1, and has the function of delivering a substance into a cell, and
(d) a polypeptide that comprises an amino acid sequence encoded by a polynucleotide hybridizing under highly stringent conditions to a polynucleotide encoding the amino acid sequence of SEQ ID NO: 1, and has the function of delivering a substance into a cell.
[2] The polypeptide according to [1], wherein the polypeptide has a function of delivering a substance into the nucleus.
[3] The polypeptide according to [1] or [2], wherein the cell is a mammalian cell.
[4] A complex of the polypeptide according to any one of [1] to [3] and a substance that functions intracellularly.
[5] The complex according to [4], wherein the polypeptide according to any one of [1] to [3] and the substance that functions intracellularly are bound through a covalent bond and/or a non-covalent bond.
[6] The complex according to [4] or [5], wherein the substance that functions intracellularly is selected from a toxin, an enzyme, a cell growth-inhibiting substance, an inhibitory nucleic acid molecule, a genome editing molecule, an antibody or an antigen binding fragment thereof, a signal transduction-regulating substance, a transcriptional factor, a gene and a label.
[7] A fusion polypeptide comprising, in a single molecule, the polypeptide according to any one of [1] to [3] and a polypeptide that functions intracellularly.
[8] A composition for use in delivering a substance into a cell, comprising the polypeptide according to any one of [1] to [3].
[9] A composition for use in treating a disease that is improved by the delivery of a substance that functions intracellularly into a cell, comprising the complex according to any one of [4] to [6] or the polypeptide according to [7].
[10] A method of delivering a substance that functions intracellularly into a cell, comprising the step of contacting a complex of the substance and the polypeptide according to any one of [1] to [3] with the cell.
[11] A polypeptide having a function of degrading a nucleic acid molecule, the polypeptide being selected from
(e) a polypeptide comprising the amino acid sequence of SEQ ID NO: 2,
(f) a polypeptide that comprises an amino acid sequence containing a mutation of one or more amino acids in the amino acid sequence of SEQ ID NO: 2, and has the function of degrading a nucleic acid molecule,
(g) a polypeptide that comprises an amino acid sequence having 80% or higher sequence identity to the amino acid sequence of SEQ ID NO: 2, and has the function of degrading a nucleic acid molecule, and
(h) a polypeptide that comprises an amino acid sequence encoded by a polynucleotide hybridizing under highly stringent conditions to a polynucleotide encoding the amino acid sequence of SEQ ID NO: 2, and has the function of degrading a nucleic acid molecule.
[12] A complex of the polypeptide according to [11] and a substance that promotes the translocation of the polypeptide into a cell.
[13] The complex according to [12], wherein the polypeptide according to [11] and the substance that promotes the translocation of the polypeptide into a cell are bound through a covalent bond and/or a non-covalent bond.
[14] The complex according to [12] or [13], wherein the substance that promotes the translocation of the polypeptide into a cell is selected from the polypeptide according to any one of [1] to [3], a cell-penetrating peptide, a cell binding domain of a proteinous toxin, a virus vector and a non-viral vector.
[15] A fusion polypeptide comprising, in a single molecule, the polypeptide according to [11] and a polypeptide that promotes the translocation of the polypeptide according to [11] into a cell.
[16] A composition for use in treating a disease that is improved by the degradation of a nucleic acid molecule, comprising the polypeptide according to [11] or [15] or the complex according to any one of
[12] to [14].
[17] A method of degrading a nucleic acid molecule, comprising the step of allowing the polypeptide according to [11] or [15] or the complex according to any one of [12] to [14] to act on the nucleic acid molecule.
[18] A polypeptide having a function of degrading a nucleic acid molecule and a function of translocating into the nucleus, the polypeptide being selected from
(i) a polypeptide comprising the amino acid sequence of SEQ ID NO: 3,
(j) a polypeptide that comprises an amino acid sequence containing a mutation of one or more amino acids in the amino acid sequence of SEQ ID NO: 3, and has the function of degrading a nucleic acid molecule and the function of translocating into the nucleus,
(k) a polypeptide that comprises an amino acid sequence having 80% or higher sequence identity to the amino acid sequence of SEQ ID NO: 3, and has the function of degrading a nucleic acid molecule and the function of translocating into the nucleus, and
(l) a polypeptide that comprises an amino acid sequence encoded by a polynucleotide hybridizing under highly stringent conditions to a polynucleotide encoding the amino acid sequence of SEQ ID NO: 3, and has the function of degrading a nucleic acid molecule and the function of translocating into the nucleus.
[19] A complex of the polypeptide according to [18] and a targeting molecule.
[20] A composition for use in damaging a cell, comprising the polypeptide according to [18] or the complex according to [19].
[21] A method of damaging a cell, comprising the step of contacting the polypeptide according to [18] or the complex according to [19] with the cell.
[22] A polynucleotide encoding the polypeptide according to any one of [1] to [3], [7], [11], [15] and [18].
[23] A vector comprising the polynucleotide according to [22].
[24] A transformed cell comprising the polynucleotide according to [23].
[25] A method of producing the polypeptide according to any one of [1] to [3], [7], [11], [15] and [18], comprising the step of culturing the transformed cell according to [24] under conditions suitable for the expression of the polypeptide according to any one of [1] to [3], [7], [11], [15] and [18].

The intracellular delivery polypeptide according to the present disclosure provides a new option for transporting a substance from the outside of a cell into the cytoplasm and further into the nucleus. The nucleic acid-degrading polypeptide according to the present disclosure provides a new option for suppressing cell growth or treating a disease related to cell growth.

### BRIEF DESCRIPTION OF DRAWINGS

FIG. 1 is a graph in which the influence of a temperature on the toxicity of sponge extracts was evaluated. The ordinate depicts a survival rate (%), and the abscissa depicts incubation time (h). “Control” depicts untreated control, and “No heat” depicts crude extracts without heat treatment.

FIG. 2 is a graph in which the toxicity of a captured fraction (Retentate) and a flow-through fraction (Filtrate) was evaluated when crude extracts were subjected to ultrafiltration. The ordinate depicts survival rate (%), and the abscissa depicts incubation time (h). “Control” depicts untreated control.

FIG. 3 is a diagram showing difference in the color of extracts between the presence and absence of kojic acid added. 1 depicts extracts obtained with a buffer without kojic acid, and 2 depicts extracts obtained with a buffer supplemented with 0.5% w/v kojic acid.

FIG. 4 is a diagram showing difference in the toxicity of extracts to brine shrimp between the presence and absence of kojic acid added.

FIG. 5 is a graph in which the influence of pH on the toxicity of sponge extracts was evaluated. The ordinate depicts death rate (%).

FIG. 6 is a chromatogram of sponge extracts treated by hydrophobic interaction chromatography. The green region depicts a fraction found to have toxicity.

FIG. 7 is a chromatogram of sponge extracts treated by hydrophobic interaction chromatography and further treated by another hydrophobic interaction chromatography. The green region depicts a fraction found to have toxicity.

FIG. 8 is a chromatogram of sponge extracts treated by two stages of hydrophobic interaction chromatography and further treated by ion-exchange chromatography. The green region depicts a fraction found to have toxicity.

FIG. 9 is a chromatogram of sponge extracts treated by two stages of hydrophobic interaction chromatography and ion-exchange chromatography and further treated by gel filtration. The green region depicts a fraction found to have toxicity.

FIG. 10 is a graph in which Kav of fraction A was plotted on a calibration curve prepared by plotting the molecular weight of each standard substance against Kav calculated by gel filtration chromatography. The filled circles depict each standard substance, and * depicts fraction A.

FIG. 11 is an SDS-PAGE image of fraction A. Left lane depicts a molecular weight marker, and right lane depicts fraction A.

FIG. 12 is an SDS-PAGE image of a sample at each purification stage. Lane M was loaded with a molecular weight marker, lane 1 was loaded with a redissolved solution, lane 2 was loaded with a sample after hydrophobic interaction chromatography treatment, lane 3 was loaded with a sample after ion-exchange chromatography treatment, and lane 4 was loaded with a toxic component finally isolated by HPLC.

FIG. 13 is a diagram showing results of purification by HPLC. A depicts chromatogram of the first run of HPLC, B depicts SDS-PAGE image of a toxic fraction in the first run of HPLC, C depicts chromatogram of the second run of HPLC, and D depicts SDS-PAGE image of a toxic fraction in the second run of HPLC.

FIG. 14 is a chart showing results of analysis of SOR by MALDI-TOF.

FIG. 15 is a two-dimensional polyacrylamide gel electrophoresis image of SOR. Mr depicts molecular weight, and pI depicts isoelectric point.

FIG. 16 is a graph in which the cytotoxicity of SOR was evaluated. The ordinate depicts relative cell growth rate with the cell growth of an untreated control defined as 100%, and the abscissa depicts concentration of SOR.

FIG. 17 is a graph in which the toxicity of SOR to brine shrimp was evaluated. The ordinate depicts number of live individuals, and the abscissa depicts concentration of SOR. The filled circles depict number of live individuals after exposure for 24 hours, the filled squares depict number of live individuals after exposure for 36 hours, and the filled triangles depict number of live individuals after exposure for 48 hours.

FIG. 18 is a diagram showing the influence of SOR on the development of Aplysia kurodai eggs. The arrow depicts a bubble-like projection on cell surface. The scale bar represents 100 μm.

FIG. 19 is a diagram showing the state of a mouse 14 hours and 16 hours after SOR administration.

FIG. 20 is an illustrative diagram summarizing a gene sequencing approach of SOR.

FIG. 21 is a diagram showing the nucleotide sequence of a PCR product obtained by 1st strand PCR, and a predicted amino acid sequence thereof. An amino acid residue consistent with sequence information obtained by Edman degradation is indicated in red.

FIG. 22 is a diagram showing the nucleotide sequence of a PCR product obtained by miss priming 3′ RACE, and a predicted amino acid sequence thereof. An amino acid residue consistent with sequence information obtained by Edman degradation is indicated in red. The nucleic acid sequence of C-terminal 8 amino acid residues was complementary by accident with SOR4Fw2 primer.

FIG. 23 is a diagram showing the nucleotide sequence of a PCR product obtained by 3′-RACE, and a predicted amino acid sequence thereof. Amino acid residues consistent with sequence information obtained by Edman degradation are indicated in red. Four clones were obtained in 3′-RACE, and one of them is shown. In the moiety surrounded by the cyan blanket (positions 2397 to 2511 of SEQ ID NO: 51) in 3′ UTR, difference in the length and sequence of the poly-A moiety was found among the clones.

FIG. 24 is a schematic diagram summarizing inverse PCR.

FIG. 25 is a diagram showing the nucleotide sequence of a PCR product obtained by inverse PCR, and a predicted amino acid sequence thereof. Amino acid residues consistent with sequence information obtained by Edman degradation are indicated in red.

FIG. 26 is a diagram showing the nucleotide sequence of a PCR product obtained by full length PCR, and a predicted amino acid sequence thereof. Amino acid residues consistent with sequence information obtained by Edman degradation are indicated in red, and bases and amino acid residues inconsistent with a nucleotide sequence obtained by 3′-RACE are indicated in blue.

FIG. 27 is an SDS-PAGE image of liquid cell extracts and cell homogenates of E. coli transformed with a plasmid containing SOR. Lane M was loaded with a molecular weight marker, lane 1 was loaded with cell homogenates of control cells transformed with an empty vector, lane 2 was loaded with cell homogenates of cells harboring SOR, lane 3 was loaded with liquid cell extracts of control cells transformed with an empty vector, and lane 4 was loaded with liquid cell extracts of cells harboring SOR.

FIG. 28 is an electrophoresis image in which the cleavage of DNA by SOR was evaluated. Lane 1 depicts molecular weight marker, lane 2 depicts plasmid alone, lane 3 depicts plasmid+SOR, and lane 4 depicts plasmid+SOR+EDTA.

FIG. 29 is an electrophoresis image in which the cleavage of DNA by the N-terminal moiety of SOR was evaluated. Lane M depicts molecular weight marker, lane NC (negative control) depicts plasmid (pBSK+) alone, lane PC (positive control) depicts 2.5 μL of plasmid+bovine pancreatic DNaseI (1 mg/mL, Worthington Industries, Inc.), lane 1 depicts plasmid+recombinant SOR N-terminal moiety, and lane 2 depicts plasmid+recombinant SOR N-terminal moiety+EDTA.

FIG. 30 is confocal laser microscope images taken after 18 μg/mL FITC-labeled SOR or FITC-labeled BSA was allowed to act on Ba/F3 cells. The left side shows fluorescence images, and the right side shows bright field images.

FIG. 31 is confocal laser microscope images taken after 0.6 μg/mL FITC-labeled SOR was allowed to act on Ba/F3 cells. The left side shows fluorescence image, the center shows bright field image, and the right side shows merged image.

FIG. 32 is a confocal laser microscope image taken 12 hours after administration of FITC-labeled BSA to HeLa cells.

FIG. 33 is confocal laser microscope images taken 0.5 hours (left) or 1 hour (right) after administration of FITC-labeled SOR to HeLa cells.

FIG. 34 is confocal laser microscope images taken 12 hours (left) or 16 hours (right) after administration of FITC-labeled SOR to HeLa cells.

### DESCRIPTION OF EMBODIMENTS

All technical terms and scientific terms used herein have the same meanings as those usually understood by those skilled in the art, unless otherwise specified herein. All patents, applications and other publications (including online information) cited herein are incorporated herein by reference in their entirety. The present application claims the priority benefit of the Japanese application filed on Apr. 7, 2020 (Japanese Application No. 2020-069328), the contents described in the specification and drawings of which are incorporated herein.

### 1. Intracellular Delivery

In one aspect, the present disclosure provides a polypeptide having a function of delivering a substance into a cell, the polypeptide being selected from

(a) a polypeptide comprising the amino acid sequence of SEQ ID NO: 1,
(b) a polypeptide that comprises an amino acid sequence containing a mutation of one or more amino acids in the amino acid sequence of SEQ ID NO: 1, and has the function of delivering a substance into a cell,
(c) a polypeptide that comprises an amino acid sequence having 80% or higher sequence identity to the amino acid sequence of SEQ ID NO: 1, and has the function of delivering a substance into a cell, and
(d) a polypeptide that comprises an amino acid sequence encoded by a polynucleotide hybridizing under highly stringent conditions to a polynucleotide encoding the amino acid sequence of SEQ ID NO: 1, and has the function of delivering a substance into a cell (hereinafter, also referred to as the “intracellular delivery polypeptide of the present disclosure”).

In the polypeptide (b), the mutation of amino acid(s) includes deletion, a substitution and/or an addition of amino acid(s). The polypeptide (b) can contain a mutation of, for example, but is not limited to, 1 to 150, 1 to 145, 1 to 140, 1 to 135, 1 to 130, 1 to 125, 1 to 120, 1 to 115, 1 to 110, 1 to 105, 1 to 100, 1 to 95, 1 to 90, 1 to 85, 1 to 80, 1 to 75, 1 to 70, 1 to 65, 1 to 65, 1 to 63, 1 to 60, 1 to 58, 1 to 55, 1 to 53, 1 to 50, 1 to 48, 1 to 45, 1 to 43, 1 to 40, 1 to 38, 1 to 35, 1 to 33, 1 to 32, 1 to 31, 1 to 30, 1 to 29, 1 to 28, 1 to 27, 1 to 26, 1 to 25, 1 to 24, 1 to 23, 1 to 22, 1 to 21, 1 to 20, 1 to 19, 1 to 18, 1 to 17, 1 to 16, 1 to 15, 1 to 14, 1 to 13, 1 to 12, 1 to 11, 1 to 10, 1 to 9 (1 to several), 1 to 8, 1 to 7, 1 to 6, 1 to 5, 1 to 4, 1 to 3, 1 to 2, or 1 amino acid(s) in the amino acid sequence of SEQ ID NO: 1. In general, a fewer number of mutations is more preferred.

The polypeptide (c) can have 80% or higher, 81% or higher, 82% or higher, 83% or higher, 84% or higher, 85% or higher, 86% or higher, 87% or higher, 88% or higher, 89% or higher, 90% or higher, 91% or higher, 92% or higher, 93% or higher, 94% or higher, 95% or higher, 96% or higher, 97% or higher, 98% or higher, 99% or higher, 99.1% or higher, 99.2% or higher, 99.3% or higher, 99.4% or higher, 99.5% or higher, 99.6% or higher, 99.7% or higher, 99.8% or higher, or 99.9% or higher sequence identity to the amino acid sequence of SEQ ID NO: 1. In general, higher sequence identity is more preferred.

In the present disclosure, the “polynucleotide hybridizing under highly stringent conditions” refers to, for example, a polynucleotide that is obtained by using colony hybridization, plaque hybridization or Southern hybridization, etc., with the whole or a portion of a polynucleotide consisting of a nucleotide sequence complementary to a reference nucleotide sequence as a probe. A method described in, for example, Sambrook and Russell, Molecular Cloning: A Laboratory Manual Vol. 4, Cold Spring Harbor, Laboratory Press 2012, Ausubel, Current Protocols in Molecular Biology, John Wiley & Sons 1987-1997 can be used as a hybridization method.

In the present disclosure, the “highly stringent conditions” are, for example, but are not limited to, conditions involving 5×SSC, 5×Denhardt's solution, 0.5% SDS, 50% formamide, and 50° C., or 0.2×SSC, 0.1% SDS, and 60° C., or 0.2×SSC, 0.1% SDS, and 62° C., or 0.2×SSC, 0.1% SDS, and 65° C. Under these conditions, the elevation of the temperature can be expected to efficiently produce DNA having higher sequence identity. However, possible factors that influence the stringency of hybridization are a plurality of factors such as a temperature, a probe concentration, a probe length, ionic strength, a time, and a salt concentration. Those skilled in the art are capable of achieving similar stringency by appropriately selecting these factors.

In the case of using a commercially available kit in hybridization, for example, Alkphos Direct Labelling and Detection System (GE Healthcare) can be used. In this case, hybridized DNA can be detected after overnight incubation with a labeled probe followed by washing of membrane with a primary washing buffer containing 0.1% (w/v) SDS under a condition of 55 to 60° C. according to a protocol attached to the kit. Alternatively, when a probe is prepared on the basis of the whole or a portion of a nucleotide sequence complementary to the nucleotide sequence of SEQ ID NO: 1, or a nucleotide sequence complementary to a nucleotide sequence encoding the amino acid sequence of SEQ ID NO: 3 or 4, the probe may be labeled with digoxigenin (DIG) using a commercially available reagent (e.g., PCR Labeling Mix (Roche Diagnostics GmbH)). In this case, hybridization can be detected using DIG Nucleic Acid Detection Kit (Roche Diagnostics GmbH).

Other examples of the polynucleotide capable of hybridizing can include a polynucleotide having 60% or higher, 61% or higher, 62% or higher, 63% or higher, 64% or higher, 65% or higher, 66% or higher, 67% or higher, 68% or higher, 69% or higher, 70% or higher, 71% or higher, 72% or higher, 73% or higher, 74% or higher, 75% or higher, 76% or higher, 77% or higher, 78% or higher, 79% or higher, 80% or higher, 81% or higher, 82% or higher, 83% or higher, 84% or higher, 85% or higher, 86% or higher, 87% or higher, 88% or higher, 89% or higher, 90% or higher, 91% or higher, 92% or higher, 93% or higher, 94% or higher, 95% or higher, 96% or higher, 97% or higher, 98% or higher, 99% or higher, 99.1% or higher, 99.2% or higher, 99.3% or higher, 99.4% or higher, 99.5% or higher, 99.6% or higher, 99.7% or higher, 99.8% or higher, or 99.9% or higher sequence identity to the nucleotide sequence of SEQ ID NO: 1 by calculation with BLAST homology search software using default parameters.

The sequence identity of an amino acid sequence or a nucleotide sequence can be determined using algorithm BLAST by Karlin and Altschul (Basic Local Alignment Search Tool) (Proc Natl Acad Sci USA. 1990; 87(6):2264-8, 1990, Proc Natl Acad Sci USA. 1993; 90(12):5873-7). In the case of using BLAST, default parameters of each program are used.

In the present disclosure, the “function of delivering a substance into a cell” means a function by which a polypeptide transfers the substance attached thereto into a cell. The delivery into a cell includes delivery from the outside of the cell into the cytoplasm, delivery from the inside of the cytoplasm into the nucleus, and delivery from the outside of the cell via the inside of the cytoplasm into the nucleus. Whether or not a polypeptide has the function of delivering a substance into a cell can be determined, for example, by binding a detectable label (e.g., a fluorescent label such as FITC) to the polypeptide, contacting the labeled polypeptide with the cell, and observing the localization of the label after a lapse of a predetermined time (e.g., 1 to 24 hours). In case the label is localized in the cell (in the cytoplasm or in the nucleus) after a lapse of a predetermined time, the polypeptide has the function of delivering a substance into a cell.

The cell into which the intracellular delivery polypeptide of the present disclosure delivers the substance is not particularly limited and includes prokaryotic or eukaryotic cells, for example, microbial (e.g., bacterial, fungal, and yeast) cells, animal cells, and plant cells. Examples of animal cells include mammalian, avian, reptilian, amphibian, and fish cells. Examples of the mammalian cells include human, nonhuman primate, rodent (e.g., mouse, rat, and guinea pig), dog, cat, bovine, horse, pig, goat, sheep, and rabbit cells.

The substance to be delivered by the intracellular delivery polypeptide of the present disclosure is not particularly limited and includes atoms, molecules, compounds, proteins, nucleic acids, and the like. In one embodiment, the substance to be delivered by the intracellular delivery polypeptide of the present disclosure is a substance that functions intracellularly. Examples of the substance that functions intracellularly include, but are not limited to, toxins, enzymes, enzyme inhibitors, receptor inhibitors, dominant negative mutants, cell growth-inhibiting substances, inhibitory nucleic acid molecules, genome editing molecules, transcriptional factors, antibodies and antigen binding fragments thereof, signal transduction-regulating substances, genes and labels.

The inhibitory nucleic acid molecule means a nucleic acid molecule that inhibits the expression of a target molecule and includes RNAi molecule, microRNA, microRNA mimic, piRNA (Piwi-interacting RNA), ribozyme (see, e.g., Jimenez et al., Trends Biochem Sci. 2015; 40(11):648-661), antisense nucleic acids, and Bonac nucleic acids, for example.

The RNAi (RNA interference) molecule means any molecule having RNAi activity and encompasses, for example, but is not limited to, siRNA (small interfering RNA), shRNA (short hairpin RNA), and the like.

The siRNA refers to a small nucleic acid that has an antisense strand having complementarity with a target sequence and a sense strand having complementarity with the antisense strand, both the strands of which form a duplex at least partially.

The shRNA is a single-stranded RNA molecule containing an antisense region and a sense region having complementarity with each other, and a loop region interposed therebetween, and a duplex region is formed by the pairing of the antisense region and the sense region to assume a hairpin-like three-dimensional structure.

The microRNA mimic is a nucleic acid molecule that mimics the function of endogenous microRNA, and is well known in the art (e.g., van Rooij and Kauppinen, EMBO Mol Med. 2014; 6(7):851-64, and Chorn et al., RNA. 2012; 18(10):1796-804).

The genome editing molecule is a molecule or a molecule set capable of modifying a genome sequence in a site-specific manner. Examples thereof include, but are not limited to, molecules based on CRISPR/Cas system, TALEN, ZFN, or meganuclease (MN). The genome editing molecule based on CRISPR/Cas system functions as a set of guide RNA and Cas (CRISPR-associated protein). Examples of Cas contained in CRISPR/Cas system include, but are not limited to, Cas9, Cas12a(Cpfl), Cas12b, Cas13, xCas9, VQR, VRER, spCas9-NG, spCas9-HF1, Cas nickase (e.g., Cas9 nickase), eSpCas9, evoCas9, HypaCas9, CjCas9, Split-Cas (e.g., Split-Cas9), dCas-BE (e.g., dCas9-BE), and the like (Broeders et al., iScience. 2019; 23(1):100789). CRISPR/Cas13 can edit RNA and as such, can be used in gene silencing similar to that of RNAi.

The antibody is not particularly limited and includes IgA, IgD, IgE, IgG and IgM, including IgA1, IgA2, IgG1, IgG2a, IgG2b, IgG3 and IgG4 as subclasses. The antibody also includes IgNAR and heavy chain antibodies. The antigen binding fragment of the antibody includes antibody fragments containing the complementarity-determining regions (CDRs) of the antibody, or combinations thereof, and encompasses, for example, but is not limited to, Fab, Fab′, F(ab′)₂, Fd, Fcab, vNAR, VHH (nanobody), scFv, minibody, scFv-Fc, scFv₂ (diabody), scFv₃ (triabody), scFv₄ (tetrabody), Fv-clasp (Arimori et al., Structure. 2017; 25(10):1611-1622), BIf (bispecific scFv immunofusion, Kuo et al., Protein Eng Des Sel. 2012; 25(10):561-9), and the like.

Examples of the label include, but are not limited to, fluorescent labels, luminescent labels, radioactive labels, metal atoms detectable by MRI, etc., and compounds (e.g., complexes) containing a metal atom.

The intracellular delivery polypeptide of the present disclosure may form a complex with the substance to be delivered. Thus, the present disclosure provides a complex of the intracellular delivery polypeptide of the present disclosure and a substance to be delivered (hereinafter, also referred to as the “intracellular delivery polypeptide complex of the present disclosure”). Examples of binding mode between the intracellular delivery polypeptide of the present disclosure and the substance to be delivered in the intracellular delivery polypeptide complex of the present disclosure include, but are not particularly limited to, covalent bond and non-covalent bond (hydrogen bond, ionic bond, hydrophobic bond, disulfide bond, etc.). The intracellular delivery polypeptide of the present disclosure and the substance to be delivered may be bound directly or may be bound via an intervening element such as a linker. The linker may be degradable or nondegradable. When delivery into cytoplasm is desired, a linker that is cleaved by an intracytoplasmic enzyme or the like can be used. When delivery into the nucleus is desired, a linker that is cleaved in the nucleus without being cleaved in the cytoplasm can be used.

When the substance to be delivered is a polypeptide (e.g., a polypeptide that functions intracellularly), the intracellular delivery polypeptide complex of the present disclosure can be configured as a fusion polypeptide. Thus, the present disclosure provides a fusion polypeptide comprising the intracellular delivery polypeptide of the present disclosure and a polypeptide to be delivered in a single molecule (hereinafter, also referred to as the “intracellular delivery fusion polypeptide of the present disclosure”). The location of the polypeptide to be delivered in the fusion polypeptide is not particularly limited, and the polypeptide to be delivered is preferably located on the N-terminal side of the intracellular delivery polypeptide of the present disclosure. The fusion polypeptide can be configured such that a linker is interposed between the intracellular delivery polypeptide of the present disclosure and the polypeptide to be delivered. For the linker in the fusion polypeptide, see, for example, Chen et al., Adv Drug Deliv Rev. 2013; 65 (10): 1357-69. Hereinafter, the intracellular delivery polypeptide complex of the present disclosure encompasses the intracellular delivery fusion polypeptide of the present disclosure, unless otherwise specified.

The present disclosure also provides a composition comprising the intracellular delivery polypeptide and/or the intracellular delivery polypeptide complex of the present disclosure (hereinafter, also referred to as the “intracellular delivery polypeptide composition of the present disclosure”). This composition may comprise an additive such as a buffer, an excipient, a stabilizer, a tonicity agent, or a preservative, and a substance to be delivered, in addition to the intracellular delivery polypeptide of the present disclosure. The additive may be pharmaceutically acceptable, and the composition may be a pharmaceutical composition.

The intracellular delivery polypeptide, the intracellular delivery polypeptide complex and the intracellular delivery polypeptide composition of the present disclosure may be for delivering the substance into a cell (e.g., into the cytoplasm or into the nucleus).

The intracellular delivery polypeptide complex and the intracellular delivery polypeptide composition of the present disclosure comprising a substance that functions intracellularly may be for use in treating a disease that is improved by the delivery of the substance that functions intracellularly into a cell.

The disease that is improved by the delivery of the substance that functions intracellularly into a cell may differ depending on the substance that functions intracellularly and encompasses, for example, but is not limited to, cell proliferative disease such as cancer, infection, autoimmune disease, endocrine disease such as diabetes mellitus, neurological disease such as neurodegenerative disease, mental disease, fibrous disease such as fibrosis, cardiovascular disease, gastrointestinal disease, motor disorder, urological disease, and ophthalmologic disease.

The present disclosure also provides a method of delivering a substance into a cell, the method comprising the step of contacting the intracellular delivery polypeptide complex of the present disclosure with the cell (hereinafter, also referred to as the “intracellular delivery method of the present disclosure”). This method can be performed in vitro, ex vivo or in vivo. The contact of the intracellular delivery polypeptide complex of the present disclosure with the cell can be achieved in vitro, for example, by adding the intracellular delivery polypeptide complex of the present disclosure to a medium containing the target cell, and in vivo, for example, by administering the intracellular delivery polypeptide complex of the present disclosure to a living body containing the target cell.

The present disclosure also provides a method of treating a disease that is improved by the delivery of a substance that functions intracellularly into a cell, the method comprising the step of administering the intracellular delivery polypeptide complex or the intracellular delivery polypeptide composition of the present disclosure comprising the substance that functions intracellularly to a subject in need thereof (hereinafter, also referred to as the “treatment method A of the present disclosure”).

In the present disclosure, the “treatment” encompasses every type of medically acceptable prophylactic and/or therapeutic intervention aimed at cure, transient remission or prevention, etc., of a disease. The “treatment” encompasses medically acceptable intervention for various purposes including, for example, the delay or arrest of progression of a disease, the regression or disappearance of a lesion, the prevention of onset of the disease or the prevention of recurrence of the disease.

In the present disclosure, the “subject” means any individual organism, preferably animal, more preferably mammalian, further preferably human individual. In another embodiment, the subject may be a nonhuman animal such as a companion animal or a farm animal, particularly, a dog, a cat, a horse, a donkey, a bovine, a goat, or a sheep. The subject may be healthy (e.g., having no particular disease or not having any disease) or may be affected by some disease. When the treatment, etc., of a disease is intended, the subject typically means a subject affected by the disease or having a risk of being affected by the disease.

In this method, the intracellular delivery polypeptide complex or the intracellular delivery polypeptide composition of the present disclosure can be administered in a therapeutically effective amount. In this context, the therapeutically effective amount is an amount that prevents the onset and recurrence of the target disease, or cures the target disease.

The specific dosage of the intracellular delivery polypeptide complex or the intracellular delivery polypeptide composition of the present disclosure to be administered may be determined in consideration of the type of the substance that functions intracellularly and various conditions as to the subject in need of the administration, for example, a therapeutic purpose, a therapeutic regimen, the type of disease, the severity of symptoms, the general state of health, age, and body weight of the subject, the sex of the subject, diets, the timing and frequency of administration, a concurrent drug, responsiveness to therapy, and compliance with therapy. The total daily dose of the intracellular delivery polypeptide complex or the intracellular delivery polypeptide composition of the present disclosure is not limited and may be, for example, approximately 1 μg/kg body weight to approximately 1000 mg/kg body weight, approximately 10 μg/kg body weight to approximately 100 mg/kg body weight, or approximately 100 μg/kg body weight to approximately 10 mg/kg body weight, in terms of the amount of the intracellular delivery polypeptide. Alternatively, the dose may be calculated on the basis of the body surface area of a patient.

The administration in this method can be performed through any of various routes including both oral and parenteral routes, for example, but are not limited to, oral, intravenous, intramuscular, subcutaneous, local, intrapulmonary, intratracheal, endotracheal], intrabronchial, transnasal, transgastric, enteral, intrarectal, intraarterial, intraportal, intraventricular, intramedullary, intra-lymph node, intra-lymphatic, intracerebral, intrathecal, intracerebroventricular, transmucosal, transdermal, intranasal, intraperitoneal and intrauterine routes. The frequency of administration differs depending on the properties of the preparation or the composition used or the conditions of the subject as described above and may be, for example, a plurality of times per day (i.e., 2, 3, 4 or 5 or more times per day), once a day, every few days (i.e., every 2, 3, 4, 5, 6, or 7 days, etc.), several times a week (e.g., 2, 3, or 4 times a week), every week, or every few weeks (i.e., every 2, 3, or 4 weeks, etc.).

### 2. Nucleic Acid-Degrading Polypeptide

In another aspect, the present disclosure provides a polypeptide having a function of degrading a nucleic acid molecule, the polypeptide being selected from

(e) a polypeptide comprising the amino acid sequence of SEQ ID NO: 2,
(f) a polypeptide that comprises an amino acid sequence containing a mutation of one or more amino acids in the amino acid sequence of SEQ ID NO: 2, and has the function of degrading a nucleic acid molecule,
(g) a polypeptide that comprises an amino acid sequence having 80% or higher sequence identity to the amino acid sequence of SEQ ID NO: 2, and has the function of degrading a nucleic acid molecule, and
(h) a polypeptide that comprises an amino acid sequence encoded by a polynucleotide hybridizing under highly stringent conditions to a polynucleotide encoding the amino acid sequence of SEQ ID NO: 2, and has the function of degrading a nucleic acid molecule (hereinafter, also referred to as the “nucleic acid-degrading polypeptide of the present disclosure”).

In the polypeptide (f), the mutation of amino acid(s) includes deletion, substitution and/or addition of the amino acid(s). The polypeptide (f) can contain a mutation of, for example, but is not limited to, 1 to 62, 1 to 61, 1 to 60, 1 to 59, 1 to 58, 1 to 57, 1 to 56, 1 to 55, 1 to 54, 1 to 53, 1 to 52, 1 to 51, 1 to 50, 1 to 49, 1 to 48, 1 to 47, 1 to 46, 1 to 45, 1 to 44, 1 to 43, 1 to 42, 1 to 41, 1 to 40, 1 to 39, 1 to 38, 1 to 37, 1 to 36, 1 to 35, 1 to 34, 1 to 33, 1 to 32, 1 to 31, 1 to 30, 1 to 29, 1 to 28, 1 to 27, 1 to 26, 1 to 25, 1 to 24, 1 to 23, 1 to 22, 1 to 21, 1 to 20, 1 to 19, 1 to 18, 1 to 17, 1 to 16, 1 to 15, 1 to 14, 1 to 13, 1 to 12, 1 to 11, 1 to 10, 1 to 9(1 to several), 1 to 8, 1 to 7, 1 to 6, 1 to 5, 1 to 4, 1 to 3, 1 to 2, or 1 amino acid(s) in the amino acid sequence of SEQ ID NO: 2. In general, a fewer number of mutations is more preferred.

The polypeptide (g) can have 80% or higher, 81% or higher, 82% or higher, 83% or higher, 84% or higher, 85% or higher, 86% or higher, 87% or higher, 88% or higher, 89% or higher, 90% or higher, 91% or higher, 92% or higher, 93% or higher, 94% or higher, 95% or higher, 96% or higher, 97% or higher, 98% or higher, 99% or higher, 99.1% or higher, 99.2% or higher, 99.3% or higher, 99.4% or higher, 99.5% or higher, 99.6% or higher, 99.7% or higher, 99.8% or higher, or 99.9% or higher sequence identity to the amino acid sequence of SEQ ID NO: 2. In general, higher sequence identity is more preferred.

In the present disclosure, the “function of degrading a nucleic acid molecule” means a function by which a polypeptide cleaves the nucleic acid molecule, in the presence of a metal ion if necessary. The nucleic acid molecule encompasses DNA and RNA. Whether or not a polypeptide has the function of degrading a nucleic acid molecule can be determined, for example, by allowing the polypeptide to act on the nucleic acid molecule (e.g., plasmid DNA), if necessary, in the presence of a metal ion, and observing the size of the nucleic acid molecule after a lapse of a predetermined time (e.g., 1 to 12 hours) by, for example, agarose gel electrophoresis. In case the fragmentation of the nucleic acid molecule is found after a lapse of a predetermined time, the polypeptide has the function of degrading a nucleic acid molecule.

The nucleic acid-degrading polypeptide of the present disclosure may form a complex with a substance that promotes the translocation thereof into a cell (e.g., into the cytoplasm or into the nucleus). Thus, the present disclosure provides a complex of the nucleic acid-degrading polypeptide of the present disclosure and a substance that promotes the translocation thereof into a cell (hereinafter, also referred to as the “nucleic acid-degrading polypeptide complex of the present disclosure”). Examples of the binding mode between the nucleic acid-degrading polypeptide of the present disclosure and the substance that promotes the translocation into a cell in the nucleic acid-degrading polypeptide complex of the present disclosure include, but are not particularly limited to, covalent bond and non-covalent bond (hydrogen bond, ionic bond, hydrophobic bond, disulfide bond, etc.). The nucleic acid-degrading polypeptide of the present disclosure and the substance that promotes the translocation into a cell may be bound directly or may be bound via an intervening element such as a linker. The linker may be degradable or nondegradable. In the case of allowing the nucleic acid-degrading polypeptide of the present disclosure to act in the cytoplasm, a linker that is cleaved by an intracytoplasmic enzyme or the like can be used. In the case of allowing the nucleic acid-degrading polypeptide of the present disclosure to act in the nucleus, a linker that is cleaved in the nucleus without being cleaved in the cytoplasm can be used.

In the nucleic acid-degrading polypeptide complex of the present disclosure, the substance that promotes the translocation of the nucleic acid-degrading polypeptide of the present disclosure into a cell is not particularly limited as long as the substance is capable of promoting the translocation of a polypeptide comprising approximately 240 to approximately 380 amino acids into a cell. Examples thereof include the intracellular delivery polypeptide of the present disclosure, cell-penetrating peptides, cell binding domains of proteinous toxins, virus vectors and non-viral vectors.

Examples of cell binding domain of proteinous toxin include, but are not limited to, the B domain of AB toxin. The AB toxin is a toxin having two domains (subunits), A and B, and diphtheria toxin, Pseudomonas exotoxin A, botulinum neurotoxin, cholera toxin, anthrax toxin, Shiga toxin, pertussis toxin, E. coli heat-labile enterotoxin (LT toxin), ricin, and the like are known (Odumosu et al., Toxins (Basel). 2010; 2(7):1612-45). The B domain of the AB toxin has a function of binding to a receptor on cell membrane surface and delivering the A domain having toxicity into the cell. In a particular embodiment, the receptor binding domain of the proteinous toxin is selected from diphtheria toxin B fragment, Pseudomonas exotoxin A domain Ia and optionally domain II (Michalska and Wolf, Front Microbiol. 2015; 6:963), botulinum neurotoxin HC domain and optionally HN domain (Williams and Tsai, Curr Opin Cell Biol. 2016; 41:51-6), cholera toxin B subunit, anthrax toxin B subunit, Shiga toxin B subunit, pertussis toxin B subunit, E. coli LT toxin B subunit, and ricin B subunit.

The cell-penetrating peptide is a peptide that can pass through a cell membrane and translocate into a cell, and many types are known. Non-limiting examples of the cell-penetrating peptide include TAT, polyarginine (R5, R8, R9, etc.), Penetratin, Pep-1, proline-rich peptide (Pro), TAT-HA2, Hph-1, HP4, LAH4, LAH4-L1, Vectofusin-1, low-molecular-weight protamine (LMWP), LL-37, Pep-7, Pept1, Pept2, IVV-14, Transportant, Ig(v), pVEC, HRSV, TGN, Derived Ku-70, RW(n), RRRRRRGGRRRRG, SVS-1, L-CPP, RLW, K16ApoE, Angiopep-2, ACPP, KAFAK, hCT(9-32), VP22, and the like (see, e.g., Vanova et al., Materials (Basel). 2019; 12(17). pii: E2671, Silva et al., Biomolecules. 2019; 9(1). pii: E22, and Derakhshankhah and Jafari, Biomed Pharmacother. 2018; 108:1090-1096).

Examples of the virus vector include, but are not limited to, vectors based on adenovirus, adeno-associated virus (AAV), retrovirus, vaccinia virus, pox virus, lentivirus, herpes virus, and bacteriophages.

Examples of the non-viral vector include, but are not limited to, particulate carriers such as polymer particles, lipid particles, and inorganic particles, and bacterial vectors. Nanoparticles of nano-level size can be used as the particulate carrier. Examples of polymer particles include, but are not limited to, particles containing polymers such as cationic polymers, polyamidoamine (PAMAM), chitosan, glycol chitosan, cyclodextrin, poly(lactic-co-glycolic acid) (PLGA), poly(lactic-co-caprolactic acid) (PLCA), poly(p amino ester), and atelocollagen. Lipid particles include liposomal and non-liposomal lipid particles, for example. Liposome is a small vesicle having an internal cavity enclosed by a lipid bilayer membrane. Non-liposomal lipid particles are lipid particles having no such structure. Examples of inorganic particles include gold nanoparticles, quantum dots, silica nanoparticles, iron oxide nanoparticles (e.g., superparamagnetic iron oxide nanoparticles (SPIONs)), nanotubes (e.g., carbon nanotubes (CNTs)), nanodiamonds, and fullerene. Examples of bacterial vector include, but are not limited to, vectors based on listeria, bifidus, and salmonella. The carrier particles can be variously modified. Examples of such modification include stealthing with PEG, targeting with a targeting molecule, and modification with a cell-penetrating peptide (CPP).

When the substance that promotes the translocation into a cell is a polypeptide, the nucleic acid-degrading polypeptide complex of the present disclosure can be configured as a fusion polypeptide. Thus, the present disclosure provides a fusion polypeptide comprising the nucleic acid-degrading polypeptide of the present disclosure and a polypeptide that promotes the translocation into a cell in a single molecule (hereinafter, also referred to as the “nucleic acid-degrading fusion polypeptide of the present disclosure”). The location of the polypeptide that promotes the translocation into a cell in the fusion polypeptide is not particularly limited, and the polypeptide is preferably located on the C-terminal side of the nucleic acid-degrading polypeptide of the present disclosure. The fusion protein can be configured such that a linker is interposed between the nucleic acid-degrading polypeptide of the present disclosure and the polypeptide that promotes the translocation into a cell. For the linker in the fusion polypeptide, see, for example, Chen et al. (supra). Hereinafter, the nucleic acid-degrading polypeptide complex of the present disclosure encompasses the nucleic acid-degrading polypeptide of the present disclosure, unless otherwise specified.

The present disclosure also provides a composition comprising the nucleic acid-degrading polypeptide of the present disclosure and/or the nucleic acid-degrading polypeptide complex of the present disclosure (hereinafter, also referred to as the “nucleic acid-degrading polypeptide composition of the present disclosure”). This composition may comprise an additive such as a buffer, an excipient, a stabilizer, a tonicity agent, or a preservative; a metal ion, a substance that promotes the translocation into a cell, and others, in addition to the nucleic acid-degrading polypeptide of the present disclosure and/or the nucleic acid-degrading polypeptide complex of the present disclosure. The additive may be pharmaceutically acceptable, and the composition may be a pharmaceutical composition.

The nucleic acid-degrading polypeptide of the present disclosure and/or the nucleic acid-degrading polypeptide complex of the present disclosure may be targeted to a cell or a tissue containing the nucleic acid molecule to be degraded. The targeting can be achieved by, for example, passive targeting or active targeting. Passive targeting can be achieved by, for example, but is not limited to, enclosure in a carrier having an EPR effect (e.g., carrier particles such as a liposome, and polymer micelle). The active targeting can be achieved by, for example, but is not limited to, the addition of a targeting molecule (e.g., a cell binding domain of an antibody or a proteinous toxin) that can interact with a cell surface molecule of interest, or enclosure in a carrier with this targeting molecule added thereto.

The nucleic acid-degrading polypeptide, the nucleic acid-degrading polypeptide complex and the nucleic acid-degrading polypeptide composition of the present disclosure may be for use in treating a disease that is improved by the degradation of a nucleic acid molecule. Examples of the disease that is improved by the degradation of a nucleic acid molecule include diseases that are improved by the damage or killing of causative cells. Such a disease encompasses, for example, but is not limited to, cell proliferative disease such as cancer, infection, autoimmune disease, endocrine hyperfunction such as hyperthyroidism, fibrous disease such as fibrosis, and hyperplasia.

The present disclosure also provides a method of degrading a nucleic acid molecule, the method comprising the step of allowing the nucleic acid-degrading polypeptide, the nucleic acid-degrading polypeptide complex and/or the nucleic acid-degrading polypeptide composition of the present disclosure to act on the nucleic acid molecule (hereinafter, also referred to as the “nucleic acid degradation method A of the present disclosure”). This method can be performed in vitro, ex vivo or in vivo. This method can be performed in vitro, for example, by adding the nucleic acid-degrading polypeptide, etc., of the present disclosure to a medium containing the target nucleic acid molecule or a cell, etc., containing this molecule, and in vivo, for example, by administering the nucleic acid-degrading polypeptide, etc., of the present disclosure to a living body containing the target nucleic acid molecule. When the target nucleic acid molecule is present in a cell, it is preferred to use the nucleic acid-degrading polypeptide of the present disclosure together with a substance that promotes the translocation into a cell, or to use the nucleic acid-degrading polypeptide complex of the present disclosure. A metal ion can coexist when allowing the nucleic acid-degrading polypeptide, etc., of the present disclosure to act on the nucleic acid molecule. Examples of the metal ion include, but are not limited to, calcium ions, magnesium ions, sodium ions, and zinc ions. A metal ion that is chelated by EDTA is preferred.

The present disclosure also provides a method of treating a disease that is improved by the degradation of a nucleic acid molecule, the method comprising the step of administering the nucleic acid-degrading polypeptide of the present disclosure, the nucleic acid-degrading polypeptide complex of the present disclosure and/or the nucleic acid-degrading polypeptide composition of the present disclosure to a subject in need thereof (hereinafter, also referred to as the “treatment method B of the present disclosure”). The administration in this method is in the same manner as in the treatment method A of the present disclosure. The disease that is improved by the degradation of a nucleic acid molecule is as mentioned above.

### 3. Nuclear Translocating and Nucleic Acid-Degrading Polypeptide

In still another aspect, the present disclosure provides a polypeptide having a function of degrading a nucleic acid molecule and a function of translocating into the nucleus, the polypeptide being selected from

(i) a polypeptide comprising the amino acid sequence of SEQ ID NO: 3,
(j) a polypeptide that comprises an amino acid sequence containing a mutation of one or more amino acids in the amino acid sequence of SEQ ID NO: 3, and has the function of degrading a nucleic acid molecule and the function of translocating into the nucleus,
(k) a polypeptide that comprises an amino acid sequence having 80% or higher sequence identity to the amino acid sequence of SEQ ID NO: 3, and has the function of degrading a nucleic acid molecule and the function of translocating into the nucleus, and
(l) a polypeptide that comprises an amino acid sequence encoded by a polynucleotide hybridizing under highly stringent conditions to a polynucleotide encoding the amino acid sequence of SEQ ID NO: 3, and has the function of degrading a nucleic acid molecule and the function of translocating into the nucleus (hereinafter, also referred to as the “nuclear translocating and nucleic acid-degrading polypeptide of the present disclosure”).

In the polypeptide (j), the mutation of amino acid(s) includes deletion, substitution and/or addition of the amino acid(s). The polypeptide (j) can contain a mutation of, for example, but is not limited to, 1 to 190, 1 to 185, 1 to 180, 1 to 175, 1 to 170, 1 to 165, 1 to 160, 1 to 155, 1 to 150, 1 to 145, 1 to 140, 1 to 135, 1 to 130, 1 to 125, 1 to 120, 1 to 115, 1 to 110, 1 to 105, 1 to 100, 1 to 95, 1 to 90, 1 to 85, 1 to 80, 1 to 75, 1 to 70, 1 to 65, 1 to 60, 1 to 55, 1 to 50, 1 to 45, 1 to 40, 1 to 35, 1 to 30, 1 to 29, 1 to 28, 1 to 27, 1 to 26, 1 to 25, 1 to 24, 1 to 23, 1 to 22, 1 to 21, 1 to 20, 1 to 19, 1 to 18, 1 to 17, 1 to 16, 1 to 15, 1 to 14, 1 to 13, 1 to 12, 1 to 11, 1 to 10, 1 to 9 (1 to several), 1 to 8, 1 to 7, 1 to 6, 1 to 5, 1 to 4, 1 to 3, 1 to 2, or 1 amino acid(s) in the amino acid sequence of SEQ ID NO: 3. In general, a fewer number of mutations is more preferred.

The polypeptide (k) can have 80% or higher, 81% or higher, 82% or higher, 83% or higher, 84% or higher, 85% or higher, 86% or higher, 87% or higher, 88% or higher, 89% or higher, 90% or higher, 91% or higher, 92% or higher, 93% or higher, 94% or higher, 95% or higher, 96% or higher, 97% or higher, 98% or higher, 99% or higher, 99.1% or higher, 99.2% or higher, 99.3% or higher, 99.4% or higher, 99.5% or higher, 99.6% or higher, 99.7% or higher, 99.8% or higher, or 99.9% or higher sequence identity to the amino acid sequence of SEQ ID NO: 3. In general, higher sequence identity is more preferred.

The nuclear translocating and nucleic acid-degrading polypeptide of the present disclosure may be targeted to a cell containing a nucleic acid molecule to be degraded. In some embodiments, the targeting can be achieved by complexing the nuclear translocating and nucleic acid-degrading polypeptide of the present disclosure with a targeting molecule. Thus, the present disclosure provides a complex of the nucleic acid-degrading polypeptide of the present disclosure and a targeting molecule (hereinafter, also referred to as the “nuclear translocating and nucleic acid-degrading polypeptide complex of the present disclosure”). Examples of the binding mode between the nuclear translocating and nucleic acid-degrading polypeptide of the present disclosure and the targeting molecule in the nuclear translocating and nucleic acid-degrading polypeptide complex of the present disclosure include, but are not particularly limited to, covalent bond and non-covalent bond (hydrogen bond, ionic bond, hydrophobic bond, disulfide bond, etc.). The nuclear translocating and nucleic acid-degrading polypeptide of the present disclosure and the targeting molecule may be bound directly or may be bound via an intervening element such as a linker. The linker may be degradable or nondegradable. In one embodiment, the linker has a property of being cleaved in the cytoplasm.

In another embodiment, the targeting can be achieved by enclosing the nuclear translocating and nucleic acid-degrading polypeptide of the present disclosure in a carrier (e.g., a particulate carrier) targeted to a cell of interest. The particulate carrier is as mentioned in the section about the nucleic acid-degrading polypeptide. The targeting of the particulate carrier, particularly, to cancer cells is described in Yao et al., J Control Release. 2016 Oct. 28; 240:267-286, Yoo et al., Cancers (Basel). 2019; 11(5). pii: E640, and the like.

Any known targeting molecule can be used. Non-limiting examples of the targeting molecule to cancer include transferrin, RGD peptide, APRPG peptide, NGR peptide, F3 peptide, CGKRK peptide, folic acid, lactoferrin, glycosaminoglycan, tumor-specific antibodies (e.g., HER2 and PSMA), antigen binding fragments thereof (e.g., Fab, Fab′, F(ab′)₂, Fd, Fcab, vNAR, VHH (nanobody), scFv, minibody, scFv-Fc, scFv₂ (diabody), scFv₃ (triabody), scFv₄ (tetrabody), Fv-clasp (Arimori et al., Structure. 2017; 25(10):1611-1622), etc.), mimics thereof (e.g., monobody (adnectin), affibody, Affimer, affitin, Anticalin, Atrimer, fynomer, Armadillo repeat protein, Kunitz domain, knottin, avimer, DARPin, alphabody, O body, repebody, and the like (Simeon and Chen, Protein Cell. 2018; 9(1):3-14, Yu et al., Annu Rev Anal Chem (Palo Alto Calif.). 2017; 10(1):293-320, Wuo and Arora, Curr Opin Chem Biol. 2018 June; 44:16-22). The targeting molecule is described in Yao et al. (supra) and Yoo et al. (supra) as well as in Jahan et al., J Drug Deliv. 2017; 2017:9090325, Leng et al., J Drug Deliv. 2017; 2017:6971297, and the like.

The present disclosure also provides a composition comprising the nuclear translocating and nucleic acid-degrading polypeptide of the present disclosure and/or the nuclear translocating and nucleic acid-degrading polypeptide complex of the present disclosure (hereinafter, also referred to as the “nuclear translocating and nucleic acid-degrading polypeptide composition of the present disclosure”). This composition may comprise an additive such as a buffer, an excipient, a stabilizer, a tonicity agent, or a preservative; a metal ion, a targeting molecule, a carrier such as a particulate carrier, and others, in addition to the nuclear translocating and nucleic acid-degrading polypeptide of the present disclosure and/or the nuclear translocating and nucleic acid-degrading polypeptide complex of the present disclosure. The additive may be pharmaceutically acceptable, and the composition may be a pharmaceutical composition.

The nuclear translocating and nucleic acid-degrading polypeptide, the nuclear translocating and nucleic acid-degrading polypeptide complex and the nuclear translocating and nucleic acid-degrading polypeptide composition of the present disclosure may be for use in damaging a cell and/or for use in treating a disease that is improved by the damage of a cell. Examples of the cell to be damaged include, but are not limited to, cells causative of diseases. Non-limiting examples of such a cell encompass cancer cells, vascular endothelial cells, cancer-associated fibroblasts (CAFs), cells of pathogens such as bacteria and parasites, immunocytes, endocrine cells, and myofibroblasts. Examples of the disease that is improved by the damage of a cell include, but are not limited to, cell proliferative disease such as cancer, infection, autoimmune disease, endocrine hyperfunction such as hyperthyroidism, fibrous disease such as fibrosis, and hyperplasia.

The present disclosure also provides a method of degrading a nucleic acid molecule, the method comprising the step of allowing the nuclear translocating and nucleic acid-degrading polypeptide, the nuclear translocating and nucleic acid-degrading polypeptide complex and/or the nuclear translocating and nucleic acid-degrading polypeptide composition of the present disclosure to act on the nucleic acid molecule (hereinafter, also referred to as the “nucleic acid degradation method B of the present disclosure”. This method can be performed in vitro, ex vivo or in vivo. This method can be performed in vitro, for example, by adding the nuclear translocating and nucleic acid-degrading polypeptide, etc., of the present disclosure to a medium containing the target nucleic acid molecule or a cell, etc., containing this molecule, and in vivo, for example, by administering the nuclear translocating and nucleic acid-degrading polypeptide, etc., of the present disclosure to a living body containing the target nucleic acid molecule. A metal ion can coexist when allowing the nuclear translocating and nucleic acid-degrading polypeptide, etc., of the present disclosure to act on the nucleic acid molecule. The type of the metal ion is as described for the nucleic acid degradation method A of the present disclosure.

The present disclosure also provides a method of damaging a cell, the method comprising the step of contacting the nuclear translocating and nucleic acid-degrading polypeptide, the nuclear translocating and nucleic acid-degrading polypeptide complex and/or the nuclear translocating and nucleic acid-degrading polypeptide composition of the present disclosure with the cell (hereinafter, also referred to as the “cell damaging method of the present disclosure”). This method can be performed in vitro, ex vivo or in vivo. The contact of the nuclear translocating and nucleic acid-degrading polypeptide, etc., of the present disclosure with the cell can be achieved in vitro, for example, by adding the nuclear translocating and nucleic acid-degrading polypeptide, etc., of the present disclosure to a medium containing the target cell, and in vivo, for example, by administering the nuclear translocating and nucleic acid-degrading polypeptide, etc., of the present disclosure to a living body containing the target cell.

The present disclosure also provides a method of treating a disease that is improved by the damage of a cell, the method comprising the step of administering the nuclear translocating and nucleic acid-degrading polypeptide, the nuclear translocating and nucleic acid-degrading polypeptide complex and the nuclear translocating and nucleic acid-degrading polypeptide composition of the present disclosure to a subject in need thereof (hereinafter, also referred to as the “treatment method C of the present disclosure”). The administration in this method is in the same manner as in the treatment method A of the present disclosure. The disease that is improved by the damage of a cell is as mentioned above.

### 4. Polynucleotide, Vector and Host Cell

In yet another aspect, the present disclosure provides a polynucleotide encoding any of various polypeptides described above (i.e., each of the polypeptides (a) to (l)) (hereinafter, also referred to as the “polynucleotide of the present disclosure”). The polypeptides (a) to (l) are also collectively referred to as the “polypeptide of the present disclosure”.

The polynucleotide encompasses DNA and RNA. The DNA encompasses genome DNA, genome DNA libraries, cDNA, cDNA libraries, synthetic DNA, and the like. The polynucleotide of the present disclosure may be isolated. The nucleotide sequence of the polynucleotide of the present disclosure can be determined from the amino acid sequence of the polypeptide encoded thereby. Examples of the nucleotide sequence of the polynucleotide encoding the polypeptide (a) include a nucleotide sequence comprising SEQ ID NO: 4 or 5. Examples of the nucleotide sequence of the polynucleotide encoding the polypeptide (e) include a nucleotide sequence comprising SEQ ID NO: 6 or 7. Examples of the nucleotide sequence of the polynucleotide encoding the polypeptide (i) include a nucleotide sequence comprising SEQ ID NO: 8 or 9.

The polynucleotide of the present disclosure can be expressed by the transfection of a host cell. In this respect, the polynucleotide of the present disclosure can be codon-optimized for use in order to attain efficient expression in the host cell to be transfected. An approach of optimizing codons for host (e.g., E. coli) cells is known. Examples of the codon-optimized sequences of the polynucleotides encoding the polypeptides (a), (e) and (i) include sequences comprising SEQ ID NOs: 5, 7 and 9, respectively.

The polynucleotide of the present disclosure can be obtained by artificial synthesis or by, for example, PCR using a primer set specific for the polynucleotide of the present disclosure from the gene of a sponge of the genus Spongosorites (particularly, those living at the coast of Iriomote Island). Examples of the sequences of the polynucleotides encoding the polypeptides (a), (e) and (i) contained in the sponge of the genus Spongosorites include sequences comprising SEQ ID NOs: 4, 6 and 8, respectively.

The present disclosure also provides a vector comprising the polynucleotide of the present disclosure (hereinafter, also referred to as the “vector of the present disclosure”). Examples of the vector include library vectors, cloning vectors, and expression vectors. The expression vector is not particularly limited as long as the vector permits expression of the polynucleotide of the present disclosure in a host. Examples thereof include virus vectors and non-viral vectors. Non-limiting examples of the virus vector are as mentioned above for the nucleic acid-degrading polypeptide of the present disclosure. Non-limiting examples of the non-viral vector include those mentioned above for the nucleic acid-degrading polypeptide of the present disclosure as well as plasmid vectors, cosmid vectors, fosmid vectors, and artificial chromosome vectors such as YAC, BAC, and PAC.

In the expression vector of the present disclosure, the polynucleotide of the present disclosure may be operably linked to a promoter. Examples of the promoter include, but are not limited to, promoters for bacteria such as trc promoter, tac promoter, and lac promoter, promoters for yeasts such as GAL1 promoter, GAL10 promoter, glyceraldehyde-3-phosphate dehydrogenase promoter, and PH05 promoter, and promoters for animal cells such as SV40 early promoter and SV40 late promoter. The expression vector of the present disclosure may comprise, if desired, an enhancer, a splicing signal, a poly-A addition signal, a ribosomal binding sequence (SD sequence), a selective marker, a replication origin, and the like. Examples of the selective marker include dihydrofolate reductase gene, LacZ gene, and antibiotic resistance genes (e.g., ampicillin, neomycin, kanamycin, streptomycin, tetracycline or chloramphenicol resistance gene).

The present disclosure also provides a transformed cell comprising the polynucleotide of the present disclosure (hereinafter, also referred to as the “transformed cell of the present disclosure”). The transformed cell of the present disclosure can be obtained by transfecting a host cell with the polynucleotide of the present disclosure. The host cell is preferably a cell that can express the polynucleotide of the present disclosure. Examples thereof include, but are not limited to, cells of microbes such as bacteria (E. coli, etc.), actinomycetes, and yeasts, insect cells (SF9, etc.), and mammalian cells (e.g., human HEK293 embryonic kidney-derived cells, monkey COS cells, Chinese hamster ovary (CHO) cells, and myeloma cells). The transfection of the host cell with the polynucleotide of the present disclosure can be performed by any of various known methods, for example, a calcium phosphate method, lipofection, or electroporation. In the transformed cell of the present disclosure, the polynucleotide of the present disclosure may be contained in a vector or may be integrated in the genome of the transformed cell.

### 5. Method of Producing Polypeptide

The polypeptide of the present disclosure may be chemically synthesized by any of various synthesis methods such as a solid-phase peptide synthesis method, and typically, can be produced by allowing the transformed cell of the present disclosure to express the polynucleotide of the present disclosure. Thus, the present disclosure provides a method of producing the polypeptide of the present disclosure, the method comprising the step of culturing the transformed cell of the present disclosure under conditions suitable for the expression of the polypeptide of the present disclosure (hereinafter, also referred to as the “production method of the present disclosure”). The conditions suitable for the expression of the polypeptide of the present disclosure are typically the same as those suitable for the culture of the transformed cell and/or the parent host cell serving as a base. Non-limiting examples of the conditions suitable for the expression of the polypeptide of the present disclosure include culture at 37° C. under 5% CO₂ in a culture medium. The production method of the present disclosure may optionally comprise the step of extracting the polypeptide of the present disclosure, the step of purifying the polypeptide of the present disclosure, and other steps. The extraction and purification of the polypeptide can employ any known approach such as salting out, dialysis, or chromatography (e.g., affinity column chromatography and size exclusion chromatography). The polypeptide of the present disclosure can be produced in a form with an affinity tag such as His tag, HA tag, myc tag, or FLAG tag added thereto in order to enhance purification efficiency.

### 6. Method of Isolating Polypeptide

The present disclosure also provides a method of isolating the polypeptide of the present disclosure from a sponge of the genus Spongosorites (hereinafter, also referred to as the “isolation method of the present disclosure”).

The isolation method of the present disclosure comprises the step of obtaining aqueous extracts of the sponge of the genus Spongosorites. The step of obtaining aqueous extracts comprises subjecting a tissue of the sponge of the genus Spongosorites to extraction with an aqueous medium one to ten times, for example, 1, 2, 3, 4 or 5 times. The extraction temperature is not particularly limited as long as the temperature does not denature proteins. The temperature may be 1 to 50° C., 4 to 40° C., 10 to 30° C., 15 to 25° C. or room temperature, etc. The aqueous medium is not particularly limited as long as the aqueous extraction of proteins can be performed. Examples thereof include water, brine, and aqueous buffers (e.g., a Tris-HCl buffer). In a particular embodiment, the aqueous medium contains a tyrosinase inhibitor. Examples of the tyrosinase inhibitor include, but are not limited to, kojic acid, hydroquinone and its derivatives, deoxyarbutin and its derivatives, resorcin, resorcinol, and vanillin. Other examples of the tyrosinase inhibitor are described, for example, in Zolghadri et al., J Enzyme Inhib Med Chem. 2019; 34(1):279-309, etc.

As the sponge of the genus Spongosorites, those living preferably on the coastal ocean bed of Iriomote Island, more preferably on the ocean bed of 2 to 10 m below the coastal sea surface of Iriomote Island, particularly, the ocean bed of 3 to 8 m below the sea surface around a northern latitude of 24.35 degrees and an east longitude of 123.72 degrees, can be used. The sponge may be cryopreserved before extraction with an aqueous medium.

The isolation method of the present disclosure may optionally comprise the step of salting out the aqueous extracts, the step of fractionating the extracts by chromatography, the step of desalting the chromatography fractions, the step of evaluating the toxicity of the chromatography fractions, the step of fractionating a fraction having toxicity by chromatography, and other steps. Examples of the chromatography include, but are not limited to, hydrophobic interaction chromatography, ion-exchange chromatography, and gel filtration chromatography. In the case of performing chromatography a plurality of times, different types of chromatography methods may be combined.

Examples of the approach of evaluating toxicity include, but are not limited to, the evaluation of toxicity to brine shrimp (counting of the number of live individuals) and the evaluation of toxicity to a cell system (measurement of a survival rate), as described in Examples.

In a particular embodiment, the isolation method of the present disclosure comprises the steps of:

(i) subjecting the sponge of the genus Spongosorites to extraction with an aqueous medium containing a tyrosinase inhibitor;
(ii) salting out the aqueous extracts obtained in the step (i);
(iii) fractionating the salted-out product obtained in the step (ii) by chromatography;
(iv) evaluating the toxicity of the chromatography fractions obtained in the step (iii); and
(v) fractionating a fraction evaluated to have toxicity in the step (iv) by chromatography.
The steps (iv) to (v) may be repeated a plurality of times until a clear peak is obtained.

### EXAMPLES

Hereinafter, the present invention will be described in more detail with reference to Examples. However, the contents of the present invention are not limited by these examples.

### [Example 1] Toxicity Evaluation of Sponge Extract

Aqueous extracts of a sponge of the genus Spongosorites from Iriomote Island and gel filtration fractions thereof were evaluated for their toxicity using larvae of brine shrimp and a cancer cell system.

### <Preparation of Sample>

A sponge sample was collected from the ocean bed of 6 m below the coastal sea surface (northern latitude: 24.35 degrees, east longitude: 123.72 degrees) of Iriomote Island and cryopreserved until use in experiments. 10 g of the sample was subjected to extraction with 10 mL of water three times at room temperature to obtain crude extracts. A portion of the crude extracts was freeze-dried and subjected to gel filtration chromatography with Sephadex LH-20 open column (2.5×120 cm, GE Healthcare) (eluent: water, flow rate: 0.5 mL/min). The obtained fractions (10 mL/fraction) were combined by TLC (thin-layer chromatography, butanol:acetic acid:water=4:1:1) to obtain a total of 38 fractions.

### <Evaluation Using Brine Shrimp>

Dry eggs (commercially available product) of brine shrimp (Artemia salina) were incubated at 25° C. for 24 hours in artificial seawater (ASW). Hatched larvae were placed at a density of 10 individuals/well in a 24-well plate containing 2 mL/well of brine. 20 μL of the crude extracts or each fraction diluted, if necessary, was added to each well, and the plate was kept at 25° C. The behavior of the larvae was observed every 12 hours up to 48 hours, and the number of live individuals was counted. An individual that was totally motionless during the observation was regarded as being dead. The exertion of toxicity by the crude extracts occurred late, and the toxicity was found later than 24 hours from crude extract addition. Also, toxicity was found in the second to ninth fractions close to a void volume among the chromatography fractions. The sixth fraction and the crude extracts killed all of 10 individuals of brine shrimp used in 36 hours at a final concentration of 100 μg/mL.

### <Evaluation Using Cancer Cell System>

HT29 cells (human colorectal adenocarcinoma), A549 cells (human alveolar basal epithelial adenocarcinoma) or MDA-MB 231 cells (human breast adenocarcinoma) were inoculated at a density of 5×10³ cells/well to a 96-well plate containing a medium and incubated for 18 hours in a CO₂ incubator (37° C., 5% CO₂). The crude extracts or the sixth fraction was dissolved in a solvent of DMSO:H₂O (3:7), and added in an amount of 5 μL to each well, followed by further incubation for 72 hours. After the completion of incubation, the cells were fixed by the addition of trichloroacetic acid (50%) to each well and evaluated for cytotoxicity on the basis of an OD490 value of sulforhodamine B staining. Specifically, cytotoxicity A was determined according to the following formula.

A=100×(T−T₀)/(C−T)

wherein T₀ represents an OD value before incubation, T represents an OD value at the completion of incubation, and C represents an OD value of a control well (medium alone). A positive value of A means a cell growth inhibitory effect (the number of cells increased from that before incubation), and a negative value of A means a cytotoxic effect (the number of cells decreased from that before incubation). The results are shown in the table below. The number within the parentheses represents the concentration (μg/mL) of the extracts or the fraction.

TABLE 1
Cytotoxicity of sponge extract and gel filtration
fraction thereof

| | HT-29 | A549 | MDA-MB-231 |
|---|---|---|---|
| Crude extract | 20 (5) | −26 (5) | −1 (5) |
| Sixth fraction | 27 (25) | −1 (5) | −3 (5) |

### <Influence of Temperature on Activity>

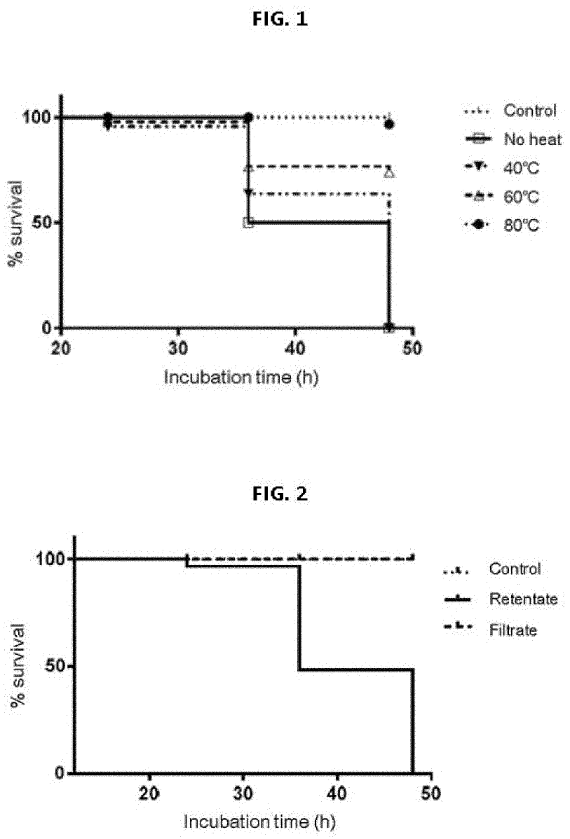

The crude extracts were heated at 40° C., 60° C. or 80° C. for 10 minutes, and toxicity to brine shrimp was evaluated in the same manner as above using these samples. As is evident from the results shown in FIG. 1, toxicity to brine shrimp disappeared by heat treatment at 80° C.

### <Size of Active Component>

The crude extracts were subjected to ultrafiltration with NMWL of 30 KDa, and a captured fraction (Retentate) and a flow-through fraction (Filtrate) were evaluated for their toxicity to brine shrimp in the same manner as above. As is evident from the results shown in FIG. 2, the size of an active component was more than 30 KDa.

### <Influence of pH on Activity>

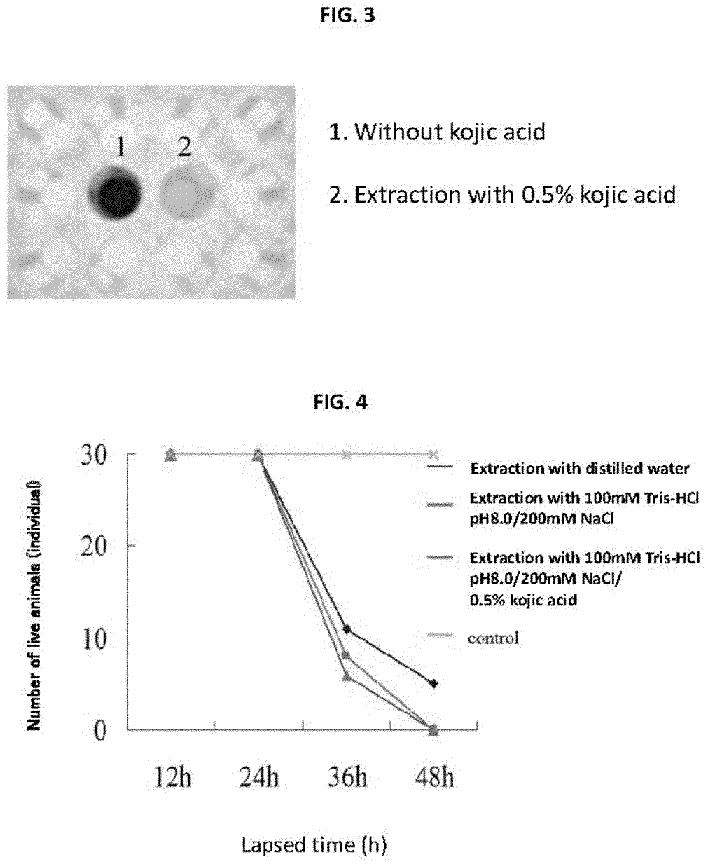

890 mg of the sponge sample was subjected to extraction with a Tris-HCl buffer (100 mM, pH 8.0) containing 200 mM NaCl and a 0.5% w/v tyrosinase inhibitor (kojic acid). The tyrosinase inhibitor was added to the extraction medium in order to suppress the production of melanin in the extracts. A tyrosinase inhibitor other than kojic acid is also substitutable. Since black discoloration was suppressed in the extracts supplemented with kojic acid (FIG. 3), the life of a column can be extended. In addition, the activity (e.g., toxicity to brine shrimp shown in FIG. 4) of the extracts also exhibited a slightly strong tendency.

HiTrap Desalting column (5 mL, GE Healthcare) was loaded with the extracts, followed by elution with 0.6 M NaCl in AKTA FPLC system (GE Healthcare) to obtain desalted crude extracts. 250 μL of the crude extracts, 50 μL each of buffers at various pH values (pH 4.0, 5.0, and 6.0: 0.5 M sodium citrate, pH 7.0: 0.5 M HEPES, pH 8.0 and 9.0: 0.5 M Tris-HCl, pH 10.0: 0.5 M glycine), and 200 μL of distilled water were mixed and injected to a 24-well plate. The plate was incubated for 2 days in an ice bath and then subjected to the same evaluation using brine shrimp as above. Life and death were determined 36 hours after addition of the animals to the wells. The results are shown in FIG. 5.

### [Example 2] Purification of Toxic Component

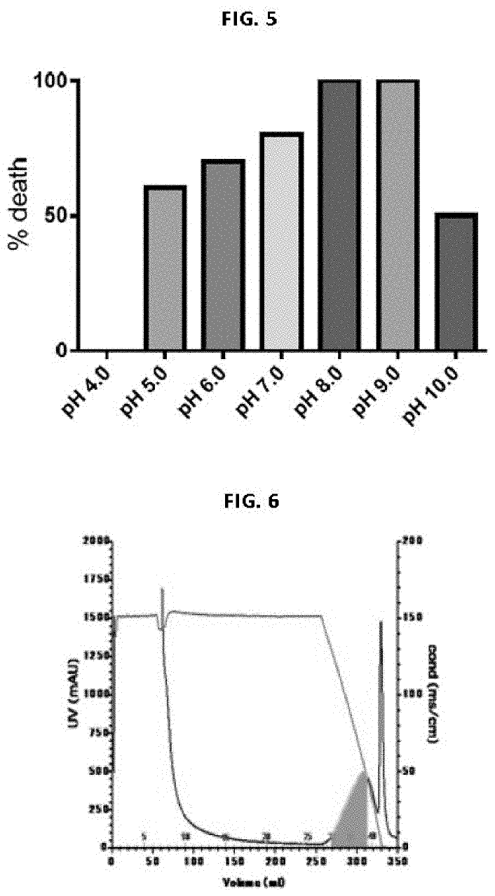

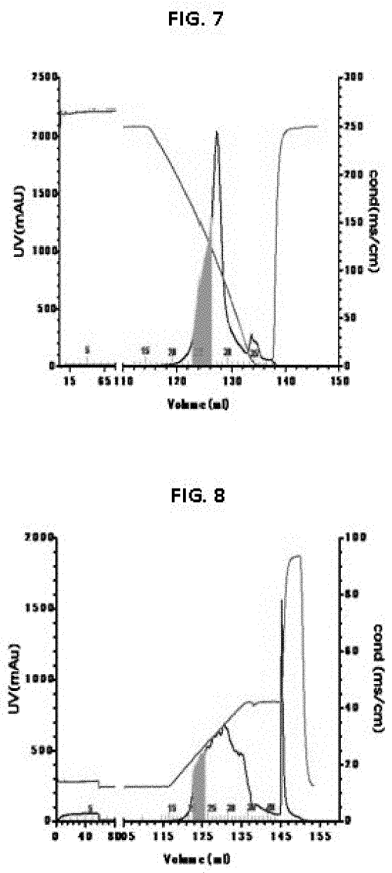

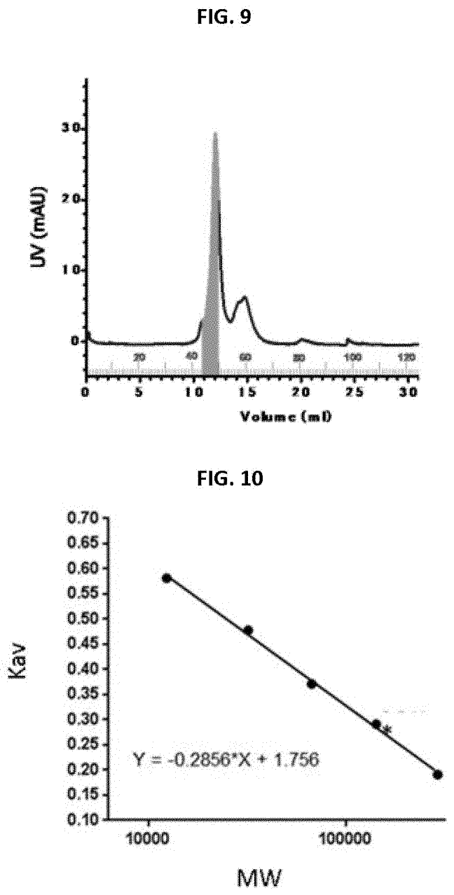

A toxic component in aqueous extracts of a sponge was purified by the following treatment. First, 100 g of a sponge sample was subjected to extraction with a Tris-HCl buffer (100 mM, pH 8.0) containing 200 mM NaCl and 0.5% w/v kojic acid. 12.5 mL of 4 M ammonium sulfate was added to 37.5 mL of the obtained crude extracts, and HiTrap Butyl FF column for hydrophobic interaction chromatography (5 mL, GE Healthcare) was loaded with the mixture. The column was washed with 1 M ammonium sulfate until the absorbance of the eluate at 280 nm reached less than 0.05 AU. Subsequently, the column was subjected to gradient elution with 1 M ammonium sulfate-water (FIG. 6), and the toxicity of each fraction thus obtained was evaluated using brine shrimp. Fractions having toxicity were combined, and HiPrep 26/10 Desalting column (53 mL, GE Healthcare) was loaded therewith for desalting, followed by elution with a Tris-HCl buffer (100 mM, pH 8.0). RESOURCE-ISO column for hydrophobic interaction chromatography (1 mL, GE Healthcare) was loaded with protein fractions, followed by elution with 2 M ammonium sulfate-water (FIG. 7). Fractions having toxicity were combined and desalted in the same manner as above, and RESOURCE-Q column for ion-exchange chromatography (1 mL, GE Healthcare) was loaded with protein fractions, followed by gradient elution with a Tris-HCl buffer (100 mM, pH 8.0, containing 0.1 to 0.4 M NaCl) (FIG. 8). Fractions having toxicity were combined and concentrated into 100 μL by ultrafiltration, and Superdex 200 10/300 GL column for gel filtration (GE Healthcare) was loaded therewith, followed by elution with a 50 mM Tris-HCl buffer (pH 9.0, containing 0.2 M NaCl). Activity was confirmed in a peak fraction at an elution volume of 12.3 mL (fraction A) (FIG. 9). AKTA FPLC system (GE Healthcare) was used in each of the chromatography methods described above.

Table 2 shows the total amount of proteins and the degree of toxicity at each stage of purification. The total amount of proteins was quantified with y globulin as a standard substance using BCA Protein Assay Kit (Thermo Fisher Scientific Inc.). One unit related to total activity represents activity that kills 50% animals within 36 hours in brine shrimp assay. The activity recovery rate represents the ratio of the total activity of fractions at each purification stage to the total activity of crude extracts.

TABLE 2
Activity at each purification stage

| | Total | | Activity | |
|---|---|---|---|---|
| | amount of | Total | recovery | Relative |
| | proteins | activity | rate | activity |
| Purification stage | (mg) | (Unit) | (%) | (Unit/mg) |
| Crude extract | 2400 | 556,000 | 100 | 230 |
| HiTrap Butyl FF | 294 | 234,000 | 42 | 796 |
| RESOURCE-ISO | 32 | 15,400 | 2.8 | 481 |
| RESOURCE-Q | 2.5 | 18,500 | 3.3 | 7370 |
| Superdex | 0.14 | 19,400 | 3.5 | 137,588 |

The molecular weight of the active component was predicted to be 145 KDa from the distribution coefficients (Kav) of the fraction A and standard substances (glutamate dehydrogenase (290 kDa), lactose dehydrogenase (142 kDa), enolase (67 kDa), myokinase (32 kDa) and cytochrome C (12.4 kDa)) calculated by gel filtration chromatography (FIG. 10).

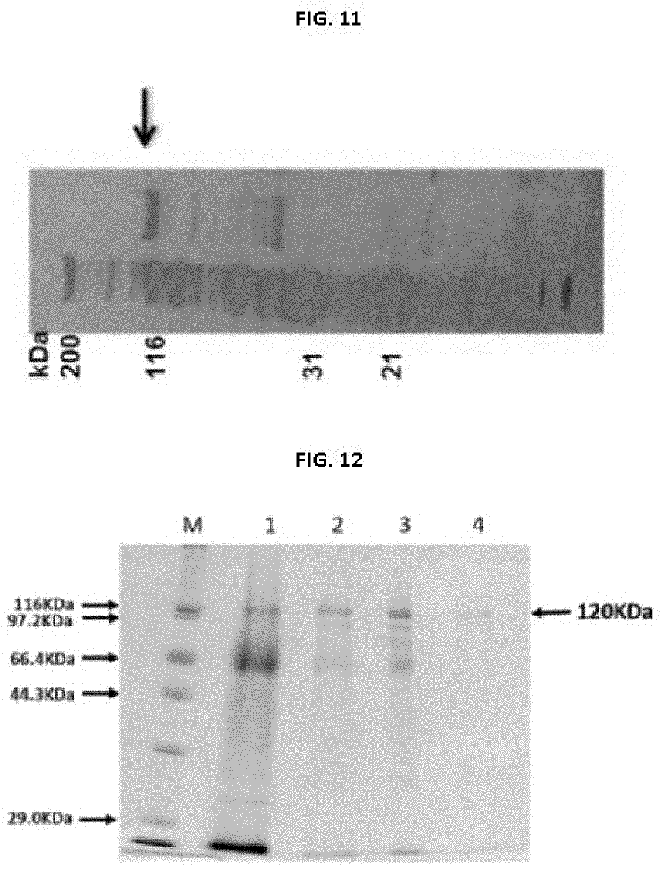

As a result of analyzing the fraction A by SDS-PAGE, the darkest band was found at a position of 120 kDa (FIG. 11, arrow in the right lane).

### [Example 3] Isolation of Toxic Component

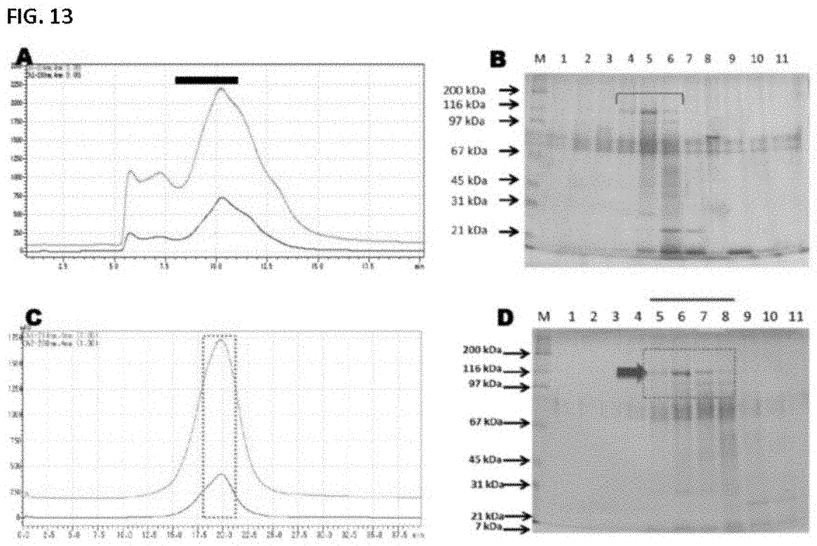

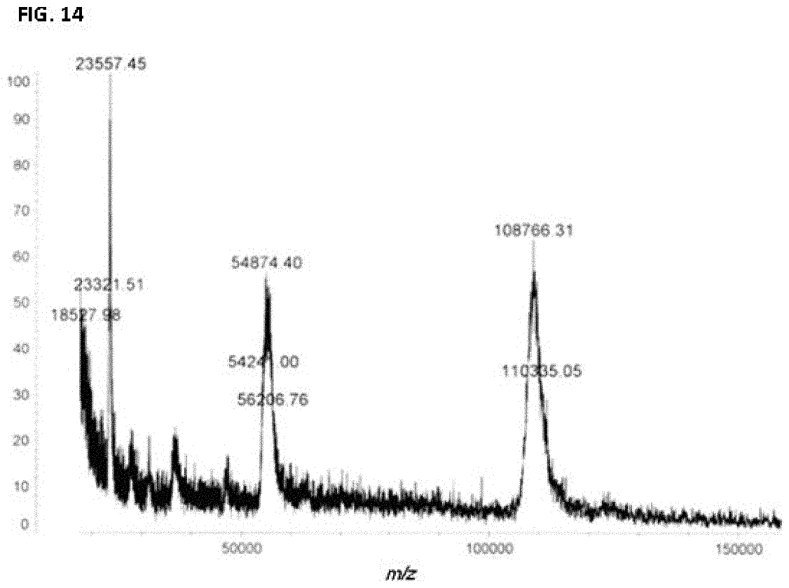

A method of purifying a toxic component was developed that permits a more rapid, large-scale process. First, 620 g of a sponge sample was subjected to extraction with a Tris-HCl buffer (100 mM, pH 8.0) containing 200 mM NaCl and 0.5% w/v kojic acid. Ammonium sulfate (final concentration: 80%) was added at 0° C. to the obtained crude extracts, and the mixture was stirred at 0° C. for 1 hour and subjected to centrifugation at 8500 rpm at 4° C. for 50 minutes. The precipitates were redissolved in a 50 mM Tris-HCl buffer (pH 8.0) containing 1 M ammonium sulfate (redissolved solution). 100 mL of the redissolved solution was added to a suspension of 100 mL of TOYOPEARL® Butyl-650 M gel (Tosoh Corp., for hydrophobic interaction chromatography, support particle diameter: 40 to 90 m) in 50 mM Tris-HCl (pH 8.0) containing 1 M sodium sulfate, followed by overnight stirring for adsorption to the gel. A column was filled with the obtained gel preparation, and the adsorbed protein was eluted with a 50 mM Tris-HCl buffer (pH 8.0) containing 0.9 M, 0.7 M and 0.2 M sodium sulfate until the absorbance of each eluate at 280 nm reached less than 0.05 AU. Active fractions eluted with the buffer containing 0.2 M sodium sulfate were dialyzed against 50 mM Tris-HCl (pH 9.0) containing 0.1 M NaCl, and RESOURCE-Q column (1 mL) was loaded therewith. The adsorbed protein was eluted in a gradient of a Tris-HCl buffer (100 mM, pH 8.0, 0.1 to 0.6 M NaCl). Final purification was performed by HPLC (pump: PU-890 (JASCO), detector: MD-4015 (JASCO), eluent: 50 mM Tris-HCl, pH 8.0, containing 0.2 M NaCl) using BioSec 5 column for gel filtration (pore size: 500 angstroms, Agilent Technologies, Inc.). FIG. 12 shows results of analyzing samples at each purification stage by SDS-PAGE. FIG. 13 shows results of purification by HPLC. Lane 4 of FIG. 12 was loaded with only the toxic component (soritesidine; also abbreviated to SOR) finally isolated by HPLC.

### [Example 4] Physicochemical Characteristics of Toxic Component

SOR isolated in Example 3 was analyzed with MALDI-TOF (AXIMA-CFR plus (Shimadzu Corp.), sinapic acid was used as a matrix). As shown in FIG. 14, the spectrum of SOR contained a cluster of singly charged ions centered at m/z=108,766 and a cluster of doubly charged ions centered at m/2z=54,874. These results also fall in with the results of SDS-PAGE of Examples 2 and 3.

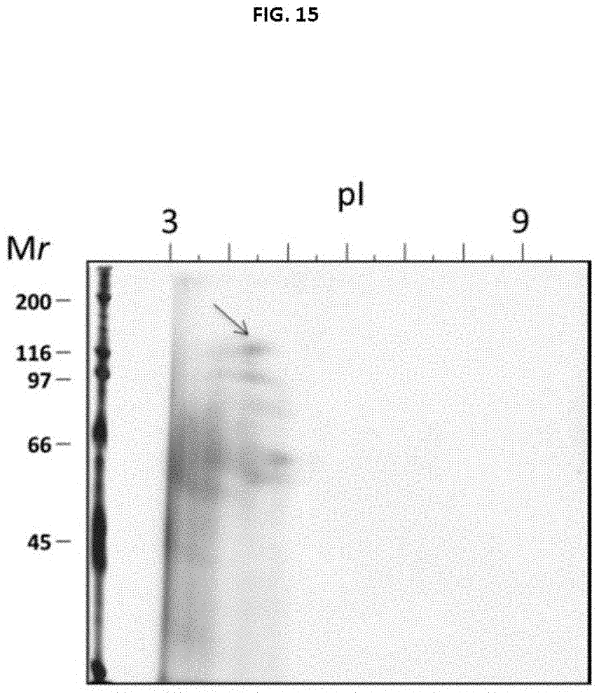

Next, the isoelectric point of SOR was determined by two-dimensional polyacrylamide gel electrophoresis (2-DE). First, 443 μg of a sample was mixed with a rehydration solution (8 M urea, 0.5% 3-[(3-cholamidopropyl)dimethylammonio]-prop anesulfonic acid (CHAPS), 0.5% Bio-Lyte 3/10 (Bio-Rad Laboratories, Inc.), 0.2% dithiothreitol, 0.002% bromophenol blue). The obtained solution was applied to IPG Strip 3-6 (Bio-Rad Laboratories, Inc.). Isoelectric focusing electrophoresis was performed at 20° C. as follows: rehydration for 12 hours, 300 V for 30 minutes, 1000 V for 30 minutes, and 5000 V for 2 hours. After electrophoresis, the strip was equilibrated for 15 minutes with shaking in 2% SDS, 50 mM Tris-HCl, pH 8.8, 6 M urea, 30% glycerol, and 0.25% DTT. Subsequently, the strip was re-equilibrated for 15 minutes with shaking in 2% SDS, 50 mM Tris-HCl, pH 8.8, 6 M urea, 30% glycerol, and 4.5% iodoacetamide. The strip was put on a 12% polyacrylamide gel, and the second electrophoresis was performed in the same manner as in SDS-PAGE. The gel was stained with a silver staining kit (Wako Pure Chemical Industries, Ltd.). As shown in FIG. 15, the band of SOR was found around an isoelectric point (pI) of approximately 4.5.

### [Example 5] Study on Bioactivity of Toxic Component

### (1) Cytotoxicity

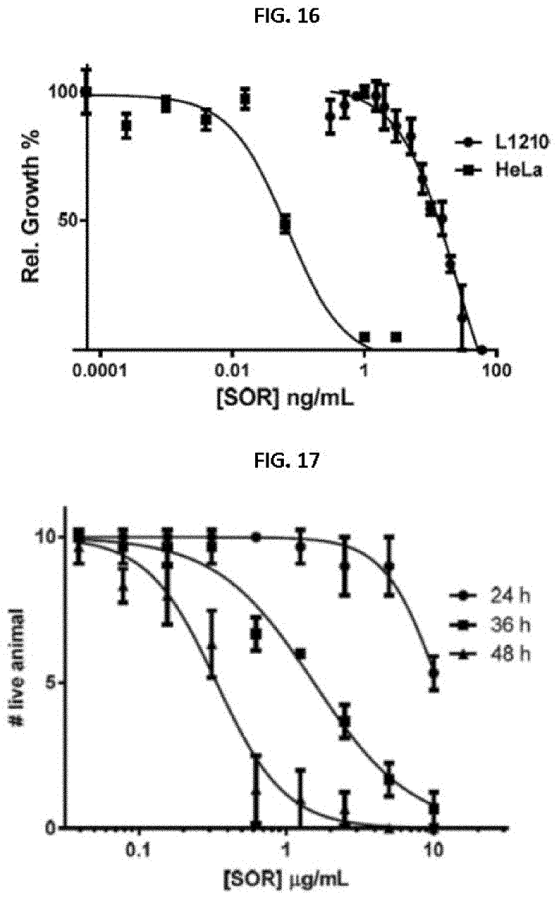

The cytotoxicity of SOR was evaluated using L1210 (RBRC-RCB2844, Riken) and HeLa (RBRC-RCB0007, Riken) cells. RPMI1640 medium supplemented with 10% FBS was used for the L1210 cells, and EMEM supplemented with 10% FBS was used for the HeLa cells. The cells were precultured for 3 days (L1210) or 7 days (HeLa) using a CO₂ incubator (37° C., 5% CO₂). For L1210, the cells that reached confluency (1×10⁵ cells/mL) were inoculated to a 96-well microtiter plate (50 L/well). SOR dissolved in a medium (200 μg/mL) was filtered (0.22 m), and serially diluted 2-fold, and the diluted products were added to wells in an amount of 50 μL/well such that the final concentration of SOR was 50 to 0.024 μg/mL. For the HeLa cells, cells that reached confluency (2×10⁴ cells/mL) were inoculated to a 96-well microtiter plate (100 μL/well) and incubated for 24 hours. SOR dissolved in a medium (200 μg/mL) was filtered (0.22 m), and serially diluted 2-fold, and the diluted products were added to wells in an amount of 10 μL/well such that the final concentration of SOR was 50 to 0.024 μg/mL. The cells of each line were incubated for 48 hours, and the growth of the cells was quantified using Cell Counting Kit-8 (Dojindo Laboratories Co., Ltd.) based on the WST-8 method according to manufacturer's protocol. Absorption at 450 nm was measured using a microplate reader, and a mean of triplicate data was plotted to prepare a concentration-response curve, followed by analysis using Prism software. As shown in FIG. 16, SOR exhibited strong cytotoxicity to both the cells (IC₅₀ value: 12.11 ng/mL (110 pM) for the L1210 cells and 0.062 ng/mL (0.517 pM) for the HeLa cells). Also, SOR exhibited selectivity approximately 200 times higher for the HeLa cells than for the L1210 cells. For reference, Table 3 shows comparison with the previously reported cytotoxicity of other compounds.

TABLE 3
Cytotoxicity of SOR

| Compound | IC₅₀ | Reference |
|---|---|---|
| SOR | 0.517 pM (HeLa) | |
| | 100 pM (L1210) | |
| Paclitaxel | 2.6 nM (HeLa) | Liebmann et al., Br J Cancer. |
| | | 1993; 68(6):1104-9 |
| Cytochalasin D | 0.1 μM (HeLa) | Miranda et al., J Cell Biol. 1974; |
| | | 61(2):481-500 |
| Swinholide A | 22 nM (L1210) | Kobayashi et al., Chem Pharm |
| | | Bull (Tokyo). 1994; 42(1):19-26 |
| Palytoxin | ~0.7 pM (A549) | Sawelew et al., BioRxiv 2018, |
| | | 292219 |
| Maito toxin | ~58 pM (smooth | Frew et al., Toxicon. 2008; 51(8): |
| | muscle cell) | 1400-8 |

### (2) Toxicity in Brine Shrimp

The LC₅₀ value of isolated SOR for brine shrimp was determined in the same manner as in Example 1. The results are shown in FIG. 17. For comparison, the LC₅₀ value was also determined 48 hours after exposure to paclitaxel, cytochalasin D and swinholide A. The results are shown in Table 4.

TABLE 4
Toxicity of SOR in brine shrimp

| Compound | LC₅₀ (μg/mL, μM) |
|---|---|
| SOR | 0.34, 0.0031 |
| Paclitaxel | 0.86, 0.99 |
| Cytochalasin D | 0.29, 0.34 |
| Swinholide A | 0.55, 0.40 |

(3) Toxicity in Aplysia kurodai Egg

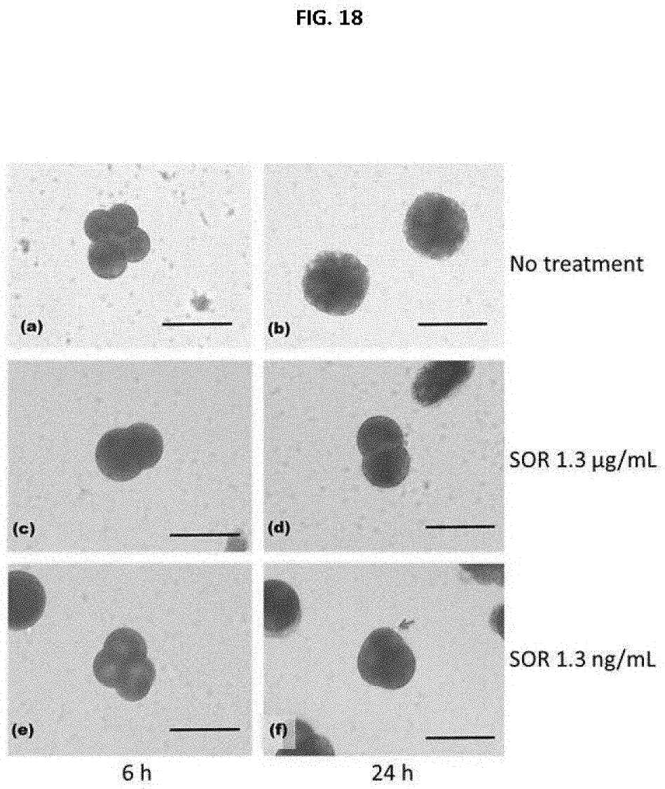

Eggs were taken out of a newly deposited egg mass of Aplysia kurodai, and influence on cleavage was examined using the collected eggs. Eggs were taken out of the egg mass in artificial seawater (ASW) and gently centrifuged by hand centrifuge apparatus. The precipitated eggs were rinsed with ASW twice. Filtered natural seawater was added to a 24-well plate (2 mL/well), to which SOR (10 μL/well) and subsequently the liquid containing the eggs (20 μL/well) were added. Then, egg development was observed under microscope every few hours. As shown in the results of FIG. 18, SOR had concentration-dependent influence on the morphology of the eggs. Untreated control eggs underwent the second cleavage 6 hours after fertilization and developed into morula in 24 hours; however, only two blastomeres were observed even 24 hours later in eggs treated with a high concentration of SOR (1.3 μg/mL), suggesting that the second cleavage was inhibited. On the other hand, in eggs treated with a high concentration of SOR (1.3 ng/mL), overall shape of the cells was shrunken and bubble-like protrusions on the cell surface were often observed, though development progressed.

### (4) Toxicity in Mice

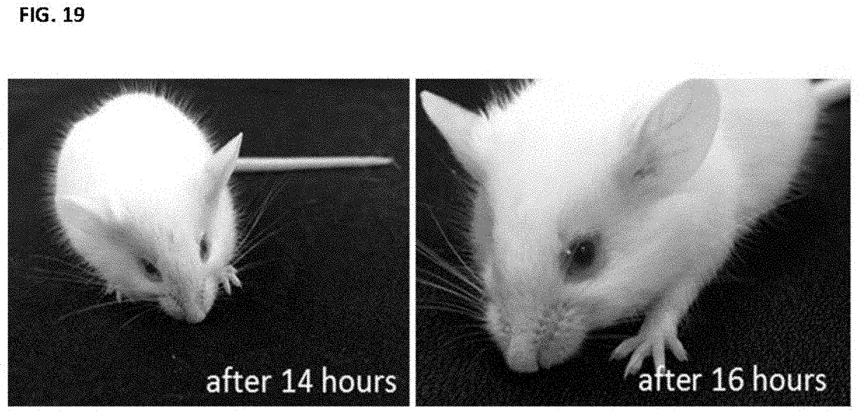

Toxicity in mice was assessed by intracerebroventricularly injecting varying concentrations of SOR to mice (ddY, male, 3 to 4 weeks old, Japan SLC, Inc.) (Sakai et al., J Pharmacol Exp Ther. 2001; 296(2):650-8). Although no acute toxicity was observed, very potent behavioral toxicity developed over time. For example, the administration of 574 ng of SOR resulted in loss of voluntary movement 14 hours later, made the mice lose their normal posture 17 hours later, and killed the mice 18 hours later. Reddish ring around eyes was observed in mice given SOR 14 hours or later after administration (FIG. 19). Administration at a dosage of 5.74 ng/mouse (1.60 pmol/kg) was also found to cause lethal toxicity by no later than 48 to 54 hours after administration. For reference, Table 5 shows comparison with the previously reported toxicity of other compounds.

TABLE 5
Toxicity of SOR in mice

| Compound | Lethal dose | References |
|---|---|---|
| SOR | 1.60 pmol/kg | |
| | (i.c.v.) | |
| Maito toxin | 37.9 pmol/kg | Yokoyama et al., J Biochem. |
| | (i.p.) | 1988; 104(2): 184-7 |
| Palytoxin | 56.0 pmol/kg | Moore and Scheuer, Science. |
| | (i.v.) | 1971; 172(3982):495-8 |
| CrTX-A | 465 pmol/kg | Nagai et al., Biochem Biophys |
| | (i.v.) | Res Commun. 2000; |
| | | 275(2):582-8 |

### [Example 6] Determination of Amino Acid Sequence of Toxic Component

(1) Determination of N-terminal amino acid sequence and amino acid sequence of digest

The N-terminal amino acid sequence of SOR was determined by Edman degradation. A semi-pure sample of SOR (hydrophobic interaction chromatography fraction at the second stage) was separated by SDS-PAGE and then blotted to PVDF membrane by electroblotting, and the membrane was stained with Ponceau S solution. A band portion of 120 kDa was excised, and the N-terminal amino acid sequence (KLGDQRQIDIASWNTFDGGVXKAN, SEQ ID NO: 10) was determined using a protein sequencer (PPSQ-21, Shimadzu Corp.).

In order to obtain an internal sequence of SOR, the band of 120 KDa described above was placed in a microtube, immersed in a very small amount of methanol, and then subjected to reduction treatment in a DTT solution. Next, 3.0 mg of monoiodoacetic acid and 10 μg of 1 M NaOH were added thereto, and the mixture was stirred for 20 minutes in the dark. The PVDF membrane was washed by immersing in pure water and subsequently 5% acetonitrile for 5 minutes. 50 μL of a Lys-C digestion solution (30% 20 mM Tris-HCl, pH 9.0, 70% acetonitrile) was added to the obtained membrane, and 1 μL of Achromobacter protease I (Wako Pure Chemical Industries, Ltd., 1 pmol/mL) was added thereto, followed by treatment at room temperature for 1 hour. The digests (50 μL) were separated by HPLC (Cadenza CD-C18 column, 0.05% TFA-acetonitrile). Then, peaks were separated, and measurement by MALDI-TOFMS was performed therefor. Fractions found to have ions were each subjected to automatic Edman degradation using a protein sequencer (PPSQ-21, Shimadzu Corp.) to determine a total 20 peptides and a total of 160 N-terminal residues, as shown in Table 6.

TABLE 6
Amino acid sequences of SOR digests

| | |
|---|---|
| XGH | SEQ ID NO: 11 |
| TGAXXXE | SEQ ID NO: 12 |
| SGXST | SEQ ID NO: 13 |
| (Q/G)AGFVPNXTXDXT | SEQ ID NO: 14 |
| NFDY | SEQ ID NO: 15 |
| LALEVPLRTVNXT | SEQ ID NO: 16 |
| GDGENXNDNXDXYD | SEQ ID NO: 17 |
| ASTGSTIPXGXXT | SEQ ID NO: 18 |
| ESAAETEN(E/G) | SEQ ID NO: 19 |
| ASAAPTNN(N/A)XTSLSSGXD(E/G) | SEQ ID NO: 20 |
| XLD(D/E)ETLE | SEQ ID NO: 21 |
| PLDVRGTY(D/E)XXXV | SEQ ID NO: 22 |
| HXQDRIQPAXPPXH | SEQ ID NO: 23 |
| ANTGIGNWIERTDNPNTVPYIPA(A/G) | SEQ ID NO: 24 |
| GXNNNHXH | |
| FAVITLGDLNADGXH | SEQ ID NO: 25 |
| LPG | SEQ ID NO: 26 |

### (2) Determination of Gene Sequence

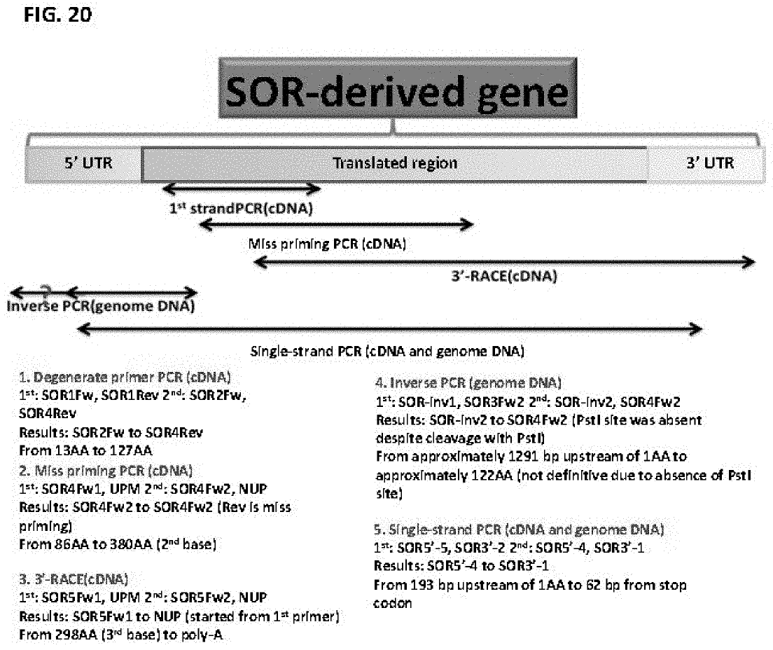

Next, an attempt was made to determine the gene sequence of SOR by use of the amino acid sequence. The sequencing is summarized in FIG. 20, and the primers used are shown in Table 7.

TABLE 7
List of primers used in sequencing

| Name | Sequence | SEQ ID NO |
|---|---|---|
| SOR1Fw | CARMGNCARATHGAYATHGCN | 27 |
| SOR1Rev | ATNGYNSWNCCNGTNSWNGC | 28 |
| SOR2Fw | TGGAAYACNTTYGAYTTYGG | 29 |
| SOR4Rev | CCNGCNGCNGGDATRTANGG | 30 |
| SOR3Fw2 | CAAATGTCACAAATCCATGTAATCCTGC | 31 |
| SOR4Fw1 | ATGATCCTACCATACTGCAAGC | 32 |
| SOR4Fw2 | GATACTGGCATTGTAGGCTCTGG | 33 |
| SOR5Fw1 | GGCACTCTCTGATCATAGACTG | 34 |
| SOR5Fw2 | CAGCTGATGAGGTATACGTCAAAGG | 35 |
| SOR-inv1 | AATCTCTTGAACCAAAATGACATCATGC | 36 |
| SOR-inv2 | CTTACATACACCGCCAAACTTGC | 37 |
| SOR3′-1 | GTGCTGGTTTTAGATGTGTGGCTTGTGC | 38 |
| SOR3′-2 | TCTGGACATATCAAGGCCCTGCAGGTGG | 39 |
| SOR5′-4 | CCATTAAACTTGAAAACATGACAGGGCC | 40 |
| SOR5′-5 | TGATCTTACAGTACAACCAGCAGCACTG | 41 |
| UPM (Long) | CTAATACGACTCACTATAGGGCAAGCAGTGGTATCAACGCAGAGT | 42 |
| UPM (Short) | CTAATACGACTCACTATAGGGC | 43 |
| NUP | AAGCAGTGGTATCAACGCAGAGT | 44 |
| rSORFw | GTCGACAAGCTTGGAGATCAGCGACAG | 45 |
| rSORRev | GAATTCCTAACAGCCAGTACCTGGAAG | 46 |

(2-1) Extraction and Purification of Total RNA, Preparation of cDNA and Extraction and Purification of Genome DNA

A sponge Spongosorites sp. collected in Iriomote Island of Okinawa, Japan in 2002 was used as a sample for cDNA preparation and for genome DNA extraction. The sponge sample was immersed in RNAlater (Qiagen N.V.) immediately after collection, preserved at −20° C. for a few days, and then preserved at −80° C. until use. Total RNA was prepared using RNeasy mini kit (Qiagen N.V.). Purified nucleic acids were assayed with NanoDrop (Thermo Fisher Scientific Inc.), and the presence or absence of the nucleic acid was confirmed by 0.8% agarose gel electrophoresis. 1^{st} strand cDNA was synthesized from the total RNA using SMARTer® RACE cDNA Amplification Kit (Takara Bio Inc.). DNA Isolation Kit ISOHAIR (Nippon Gene Co., Ltd.) was used in genome DNA extraction.

### (2-2) 1^{st} Strand PCR

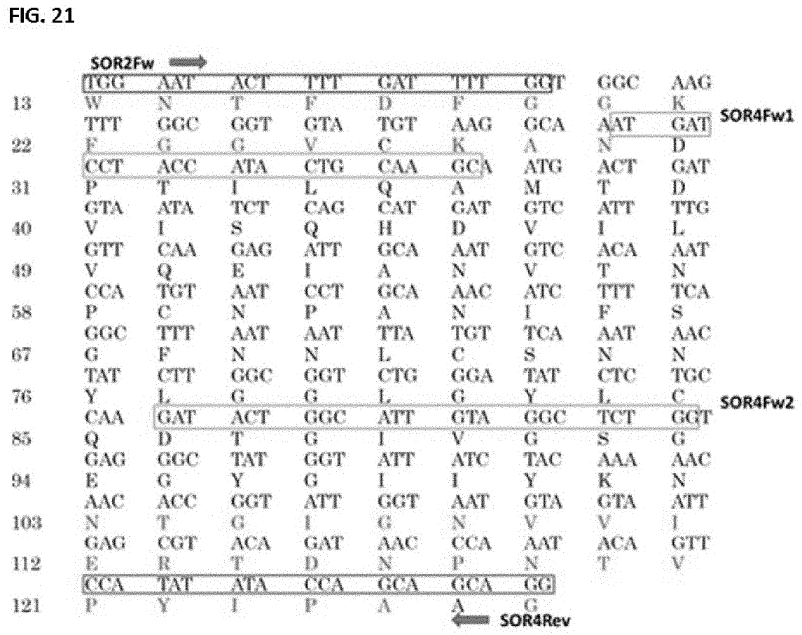

First, in order to obtain information on a short gene sequence by PCR, degenerate primers (SOR1Fw, SOR1Rev, SOR2Fw, and SOR4Rev) were prepared (Table 7) on the basis of the N-terminal amino acid sequence and internal sequence information (Table 6) obtained in the section (1). RNA was extracted from the sponge preserved in RNAlater immediately after collection, and 1^{st} strand cDNA was synthesized therefrom and used as a template in nested PCR. 1^{st} PCR was performed using SOR1Fw and SOR1Rev, and 2^{nd} a PCR was performed with the 1^{st} PCR product as a template using SOR2Fw and SOR4Rev. KOD Dash (Toyobo Co., Ltd.) was used as a PCR enzyme. The PCR product of the 2^{nd} a PCR was electrophoresed in an agarose gel. Then, a band around 300 bp was excised and purified using AxyPrep DNA Gel Extraction Kit (Axygen Biosciences, Inc.). A sequence of 115 amino acids long (SEQ ID NO: 48) corresponding to the nucleotide sequence of the obtained PCR product of 344 bp (SEQ ID NO: 47) contained a moiety well consistent with the sequence information obtained by Edman degradation (amino acid residues indicated in red in FIG. 21).

### (2-3) 3′ RACE (Miss Priming PCR)

Next, in order to obtain a 3′ total sequence, 3′ RACE was attempted. First, primers (SOR4Fw1 and SOR4Fw2) were prepared (Table 7) on the basis of the nucleotide sequence obtained above by 1^{st} strand PCR, and nested PCR was performed with 3′ RACE ready cDNA as a template. 1^{st} PCR was performed using SOR4Fw1, SOR4Fw2 and UPM (Universal Primer Mix, Clontech Laboratories, Inc.), and 2^{nd} PCR was performed with the 1^{st} PCR product as a template using SOR4Fw2 and a universal primer NUP (nested universal primer). Phusion™ High-Fidelity DNA polymerase (NEB) was used as a PCR enzyme. The PCR product of the 2^{nd} PCR was electrophoresed in an agarose gel. Then, a band around 1000 bp was excised and purified using AxyPrep DNA Gel Extraction Kit.

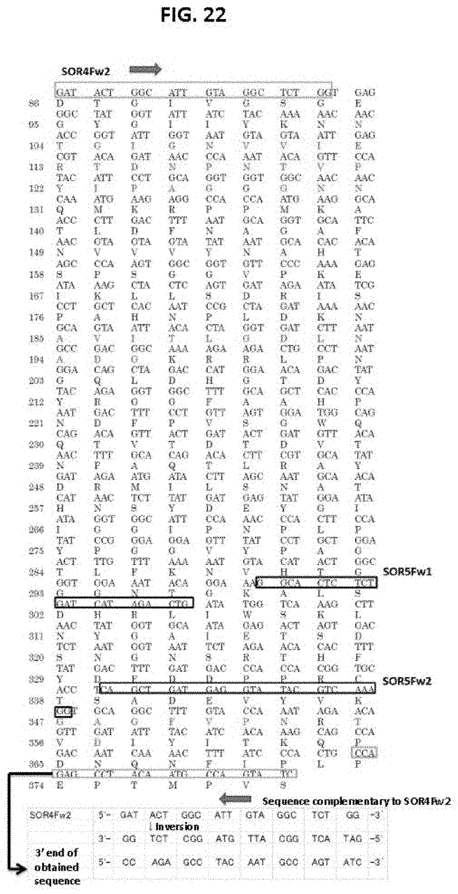

As a result, expected 3′-terminal poly-A was absent, and a PCR product of 884 bp (SEQ ID NO: 49) containing a sequence complementary to SOR4Fw2 was instead obtained. Although this might be a PCR error, this nucleotide sequence was converted, to be safe, to an amino acid sequence (SEQ ID NO: 50), which consequently contained a sequence well consistent with the amino acid sequence information obtained by amino acid sequencing in the section (1) (amino acid residues indicated in red in FIG. 22). This revealed that this PCR product resulted from the 3′ miss priming of the primer SOR4Fw2 used because the 3′ nucleotide sequence was incidentally complementary to the primer. This PCR approach is designated as “Miss priming PCR”.

### (2-4) 3′-Race

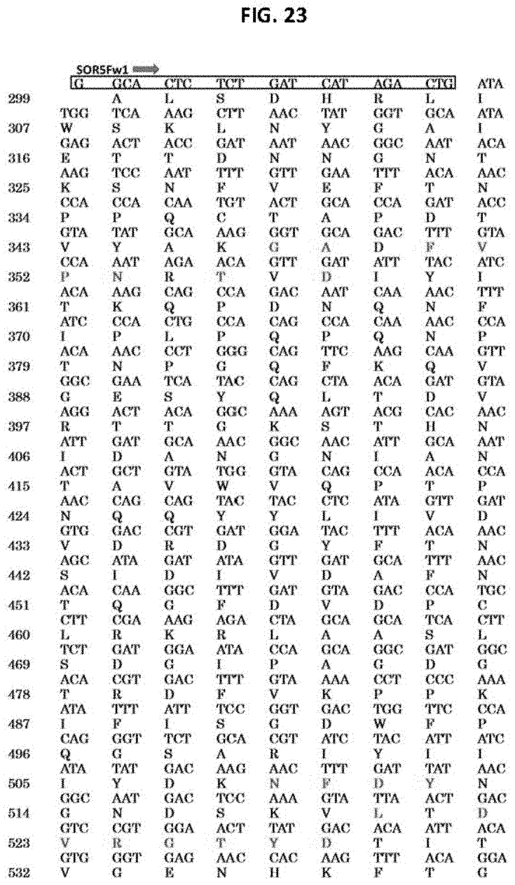

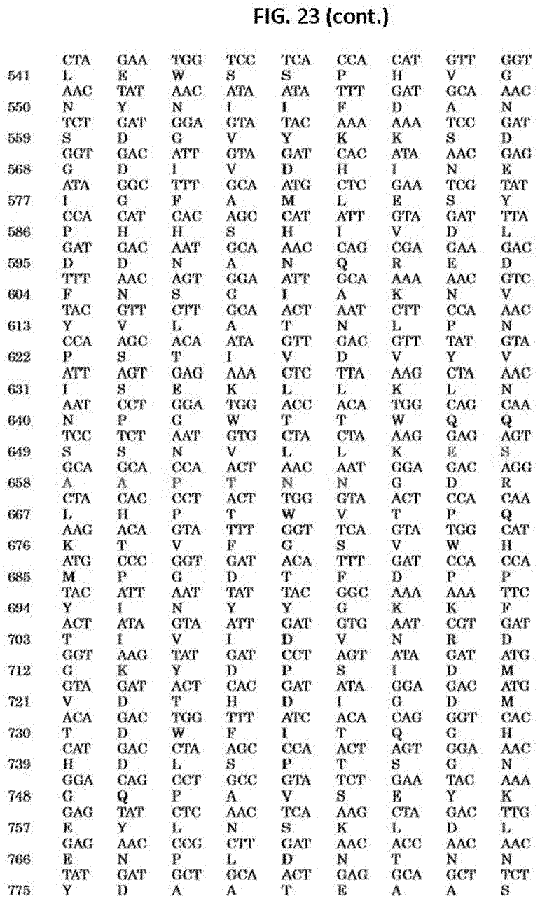

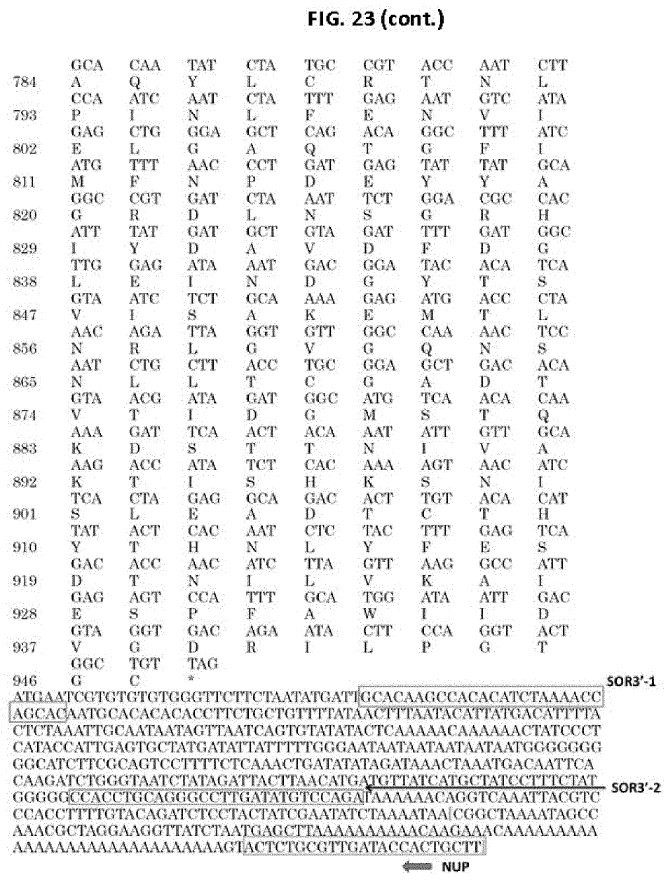

In order to amplify the 3′-terminal moiety of the SOR-derived gene by use of RACE, primers (SOR5Fw1 and SOR5Fw2) were prepared on the basis of the nucleotide sequence of 884 bp obtained by “Miss priming PCR” mentioned above, and nested PCR was performed with 3′ RACE ready cDNA as a template. 1^{st} PCR was performed using SOR5Fw1 and the same UPM as in the section (2-3), and 2^{nd} a PCR was performed with the 1^{st} PCR product as a template using SOR5Fw2 and the same NUP as in the section (2-3). Phusion™ High-Fidelity DNA polymerase (NEB) was used as a PCR enzyme. The PCR product of the 2^{nd} a PCR was electrophoresed in an agarose gel. Then, a band around 2.5 kbp was excised and purified using AxyPrep DNA Gel Extraction Kit. As a result, 4 clones of PCR products having poly-A at the 3′ end were obtained. FIG. 23 shows the sequence (SEQ ID NO: 51) of one of the clones. Some sequences were inconsistent with the sequence information obtained by Miss priming PCR, though most of sequences were consistent therewith. This nucleotide sequence was converted to an amino acid sequence (SEQ ID NO: 52), which consequently contained a sequence well consistent with the amino acid sequence information obtained by amino acid sequencing. The amino acid sequence indicated in red in the sequence information shown in FIG. 23 is the sequence well consistent with the amino acid sequencing.

### (2-5) Inverse PCR

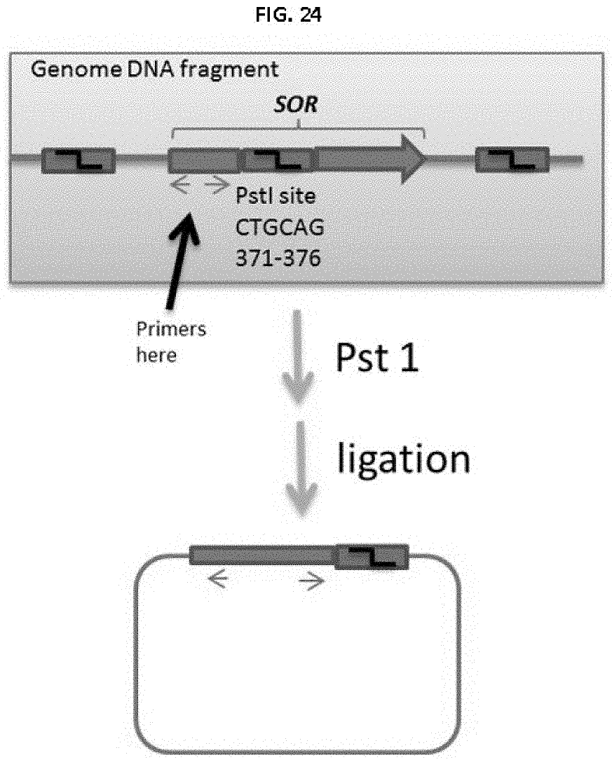

Next, in order to examine the 5′ upstream nucleotide sequence of SOR, a plurality of attempts were made to amplify the 5′-terminal sequence by use of RACE with the 1^{st} strand cDNA obtained in the section (2-1) as a template. However, no product was obtained. Accordingly, inverse PCR was performed with the genome DNA obtained in the section (2-1) as a direct template to amplify the 5′-terminal sequence of the SOR-derived gene. The inverse PCR is an approach of performing PCR by using, as a template, circular DNA obtained by the ligation of both ends of a template cleaved with a restriction enzyme, and has the advantage that the whole nucleotide sequence in the ring can be read from a portion of sequence information (FIG. 24).

First, a sample solution containing the genome DNA and a restriction enzyme PstI was prepared and incubated at 16° C. for 5 hours. Then, 10 μL of a purified product was obtained using AxyPrep PCR Clean-up Kit (Axygen Biosciences, Inc.). The product was mixed with 10 μL of Ligation Mix (Takara Bio Inc.) and self-ligated by overnight incubation at 4° C. to prepare a plasmid, which was in turn used as a template in inverse PCR.

Next, nucleic acid amplification was performed by nested PCR using primers (SOR3Fw2, SOR-inv1, and SOR-inv2) prepared on the basis of the sequence determined by 1^{st} strand PCR and SOR4Fw2 (Table 7) used in the section (2-3). Phusion™ High-Fidelity DNA polymerase (NEB) was used as a PCR enzyme. The PCR product amplified by 1^{st} PCR (SOR3Fw2 and SOR-inv1) was used as a template in 2^{nd} a PCR (SOR4Fw2 and SOR-inv2). The PCR product of the 2^{nd} a PCR was electrophoresed in an agarose gel. Then, a band around 1.5 kbp was excised and purified using AxyPrep DNA Gel Extraction Kit.

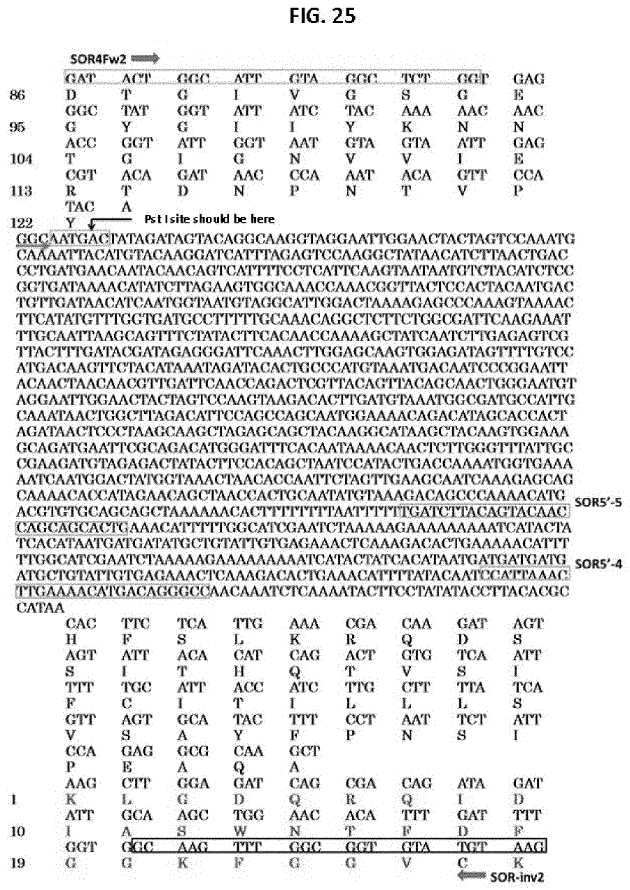

As a result, a PCR product of 1607 bp (SEQ ID NO: 53) was obtained. The nucleotide sequence information was converted to an amino acid sequence (SEQ ID NO: 54, 55), which consequently contained a sequence consistent with the sequence information obtained by amino acid sequencing in the section (1). The amino acid sequence indicated in red in the sequence information given below is the sequence consistent with the amino acid sequencing (FIG. 25). The nucleotide sequence of this PCR product was also well consistent with the nucleotide sequences of the PCR products obtained by 1^{st} strand PCR and Miss priming PCR. However, this nucleotide sequence had no PstJ digestion site to be produced during self-ligation. A nucleotide sequence up to 4 bases upstream of the PstI digestion site supposed to reside was completely consistent with the nucleotide sequences obtained from the other PCR products. The sequence indicated in red arrow in FIG. 25 is the sequence different from the other PCR products, and the sequence boxed in orange is the sequence supposed to be the PstI site. A start codon for methionine was absent before a stop codon upstream from the N-terminal amino acid sequence determined by amino acid sequencing.

### (2-6) Full Length PCR for Determining Whole Translated Region of SOR

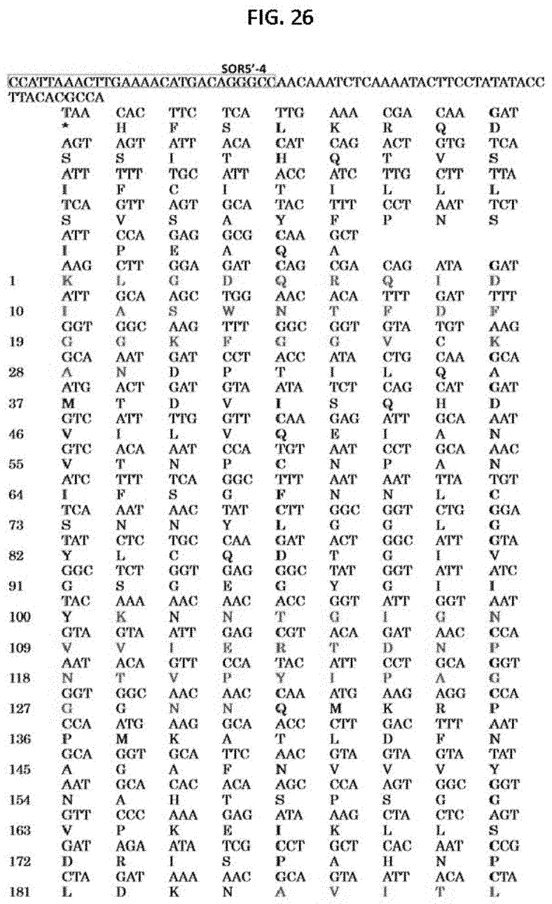

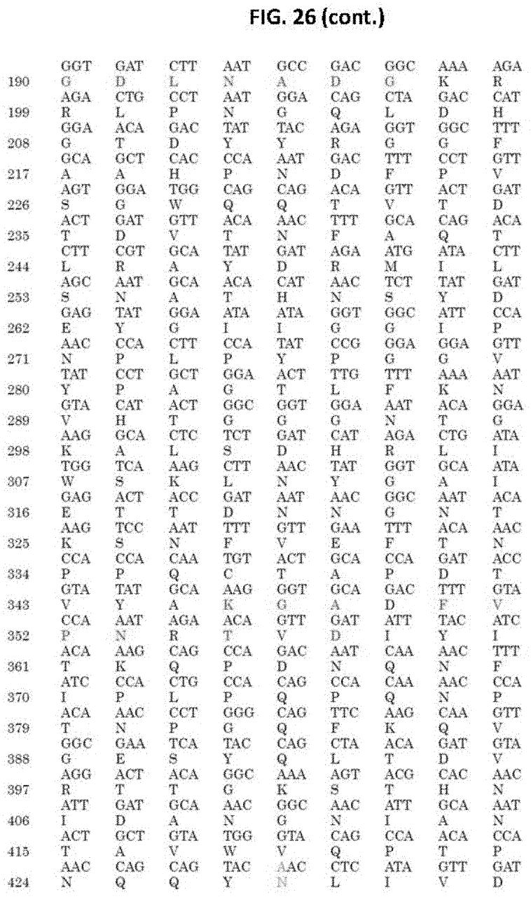

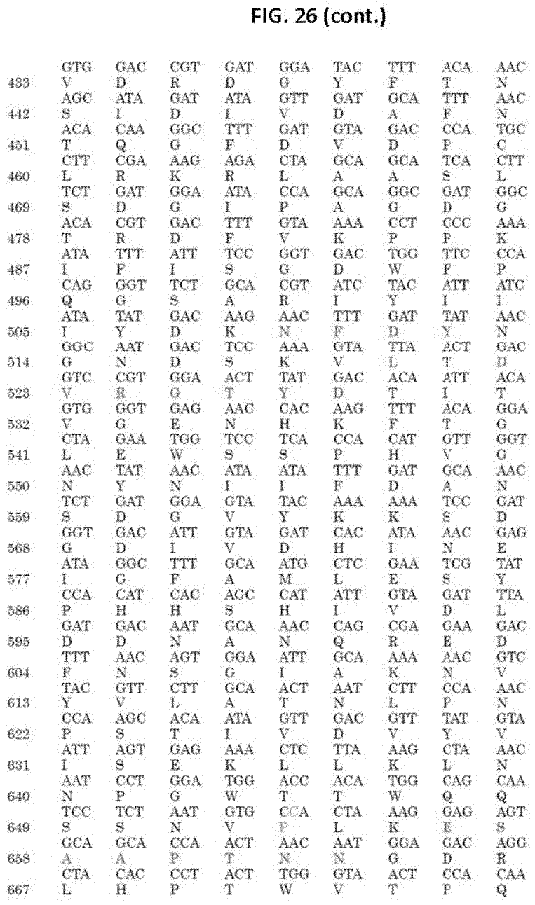

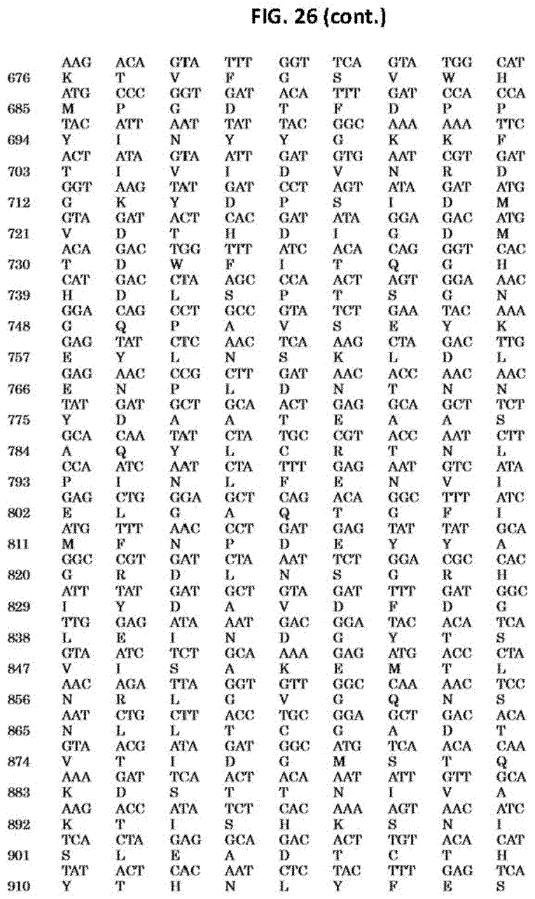

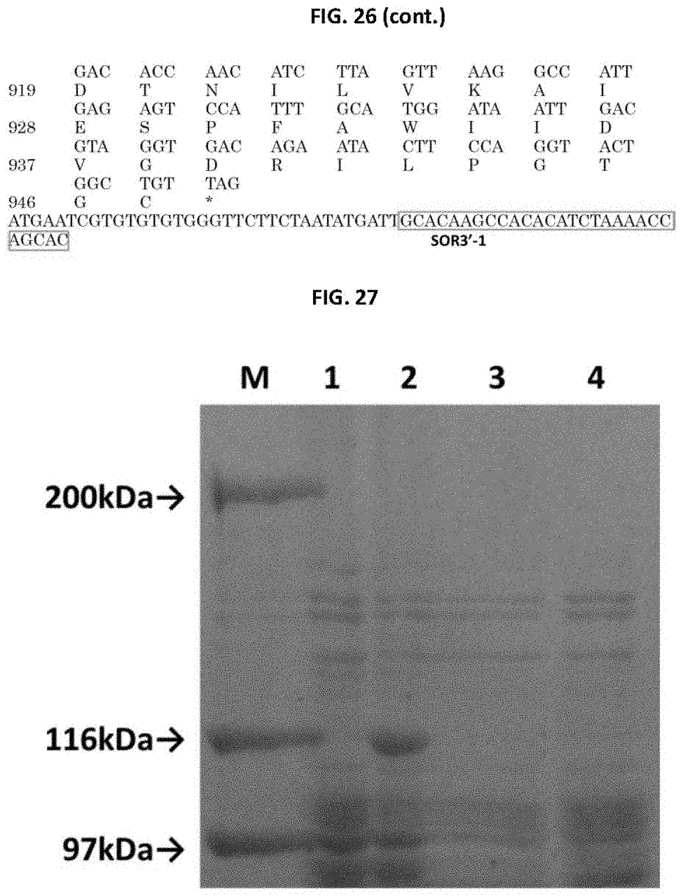

The experiments described above were able to produce fragmentary nucleotide sequence information from a plurality of PCR products, and however, presented the problems of the absence of a start codon in their respective nucleotide sequences and no restriction site confirmed in inverse PCR. Accordingly, in order to confirm the sequence information thus obtained, the gene of SOR was obtained as one consecutive nucleotide sequence. Primers (SOR5′-5, SOR3′-2, SOR5′-4, and SOR3′-1) were prepared (Table 7) for 5′ and 3′ untranslated regions on the basis of the nucleotide sequences amplified by inverse PCR and 3′-RACE. Both the 1^{st} strand cDNA and the genome DNA were used as PCR templates and each subjected to nested PCR. 1^{st} PCR was performed using SOR5′-5 and SOR3′-2, and 2^{nd} a PCR was performed with the 1st PCR product as a template using SOR5′-4 and SOR3′-1. As a result, PCR products of 3309 bp were obtained as 14 clones from the templated 1^{st} strand cDNA and as 4 clones from the templated genome DNA. One (SEQ ID NO: 56) of these clones was completely consistent between the nucleotide sequences of the amplified products from the 1^{st} strand cDNA and the genome DNA as templates except for primer-derived sequences (FIG. 26). This nucleotide sequence was well consistent with the nucleotide sequences of the other PCR methods, and however, differed by 2 bases from the nucleotide sequence obtained by 3′-RACE, and an amino acid predicted therefrom also differed (bases and amino acid indicated in blue in FIG. 26). The whole 947-amino acid sequence (SEQ ID NO: 57) of SOR was predicted by this PCR. An amino acid sequence up to N-terminal residue 311 was found to have homology with deoxyribonuclease I (DNaseI). The homology between positions 1 to 259 of bovine-derived DNaseI and positions 8 to 310 of SOR was approximately 20%. A molecular weight of 103911.07 and an isoelectric point of 4.83 were calculated from the whole amino acid sequence of the SOR translated region and were well consistent with results of preceding research. No existing protein had homology with the C-terminal amino acid sequence of SOR. The full-length amino acid sequence of SOR exhibited homology on the order of approximately 30% with the amino acid sequence of a predicted protein (GenBank Accession No. WP_022696775) of Alphaproteobacteria Euryhalocaulis caribicus.

### [Example 7] Recombinant Expression of SOR

In order to ligate the sequence of SOR with a vector for expression, recognition sites of restriction enzymes were introduced to the N terminus and C terminus of SOR by PCR using, as a template, a pTAC-1 plasmid harboring 5′-RACE ready cDNA of the SOR gene obtained in Example 6. The gene-specific primers used were rSORFw and rSORRev (Table 7) for introducing a recognition site (GTCGAC) of SalI (Takara Bio Inc.) to the N-terminal sequence of SOR and a recognition site (GAATTC) of EcoRI (Takara Bio Inc.) to the C-terminal sequence thereof. Phusion™ High-Fidelity DNA polymerase (NEB) was used as a PCR enzyme. This reaction produced rSOR.

rSOR harboring the recognition sites of the restriction enzymes and a plasmid vector pEcoli-Nterm-6×HN Vector (Clontech Laboratories, Inc.) for expression were digested with SalI and EcoRI. Specifically, 2.5 μL (15 U/μL) of SalI, 2.5 μL (15 U/μL) of EcoRI, 5.0 μL of 10×H buffer (Takara Bio Inc.), and 10 μL of rSOR/pEcoli-Nterm-6×HN Vector were each adjusted to 50 μL with dH₂O and incubated at 37° C. for 1 hour. Each restriction enzyme digestion product was subjected to agarose electrophoresis, and a band was excised and purified. After confirmation of concentrations using NanoDrop, the vector and the insert were mixed at vector:insert=1:3 to prepare a DNA solution. Subsequently, Ligation Mix and the DNA solution were mixed at Ligation Mix:DNA solution=1:1 and incubated at 25° C. for 3 hours for ligation reaction.

Competent cells NEB® 10-beta Competent E. coli (NEB) for cloning were transformed with the obtained ligation reaction solution, and plasmids were purified (see Example 6(2)). Shuffles T7 Express lysY Competent E. coli (NEB) was transformed with the purified plasmids by electroporation (see Example 6(2)), then smeared over the surface of LB agar medium containing ampicillin (100 g/mL), and cultured at 30° C. for approximately 16 hours. A very small amount of the obtained colony was picked up using a bamboo skewer, inoculated to a liquid medium containing ampicillin, and shake-cultured at 30° C. until OD₆₀₀=0.4 to 0.8. Then, IPTG (final concentration: 0.4 mM) was added thereto, followed by shake culture at 30° C. for 4 hours. The obtained culture solution was centrifuged at 6000 rpm for 10 minutes to collect E. coli, which was then suspended in a homogenization buffer (50 mM Tris-HCl, pH 7.4, 150 mM NaCl) in a volume of 1/20 times the culture solution. E. coli was lysed by repetitive sonication. The suspension was centrifuged again to obtain liquid cell extracts as a supernatant and cell homogenates as precipitates. The liquid cell extracts and the cell homogenates were subjected to SDS-PAGE. The results are shown in FIG. 27. A band around 116 kDa corresponding to SOR was found in the cell homogenates.

### [Example 8] Cleavage of DNA by Toxic Component

### (1) Natural SOR

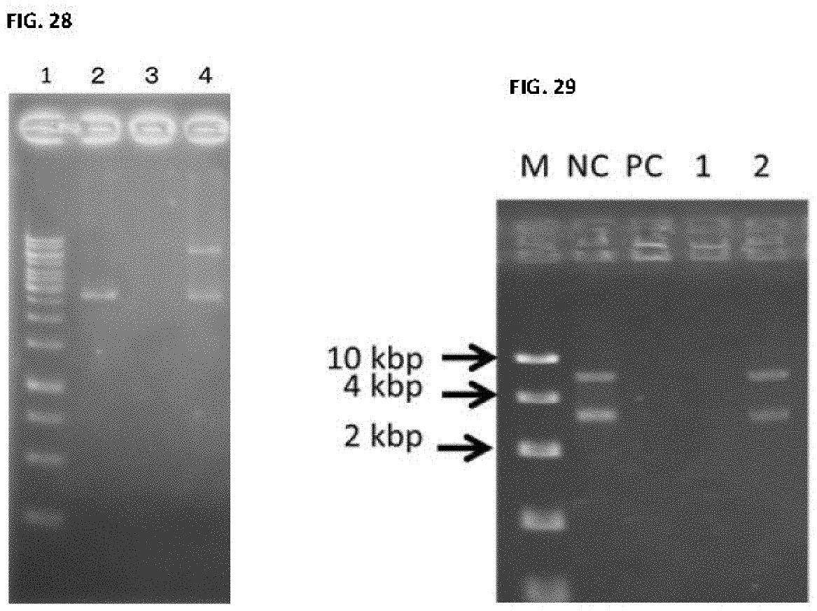

Plasmid DNA (pTAC-1, 4 ng, BioDynamics Laboratory, Inc.), or the plasmid DNA and EDTA (12.5 mM) were added to and mixed with a hydrophobic interaction chromatography fraction containing SOR (8.8 μg/mL) or a Tris-HCl buffer (pH 8.3) containing 0.1 M NaCl. The mixture was left at room temperature for 24 hours, separated by agarose electrophoresis, stained with ethidium bromide (15 min), and subsequently observed under UV light (260 nm). Thermo scientific GeneRuler 1 kb DNA Ladder (Thermo Fisher Scientific Inc.) was used as a marker. The results of FIG. 28 show that SOR cleaved DNA in a metal ion-dependent manner.

### (2) Recombinant SOR N-Terminal Moiety

DNA cleaving activity was evaluated in the same manner as in the section (1) using a recombinantly expressed polypeptide from residues 1 to 310 (SEQ ID NO: 2) of SOR. An artificial gene (rN-SOR, 930 bp (SEQ ID NO: 58), harboring a SalI recognition site (GTCGAC) on the N-terminal side and a HindIII recognition site (AAGCTT) on the C-terminal side, full-length insert sequence: 948 bp, SEQ ID NO: 59) having the gene sequence of N-terminal 310 residues of SOR optimized for E. coli codons was incorporated into pUCFa vector (2966 bp, FASMAC, SEQ ID NO: 60) having ampicillin resistance to prepare plasmid DNA (prN-SOR, 3914 bp).

NEB® 10-beta Competent E. coli (NEB) was transformed with prN-SOR by electroporation, then inoculated to LBA agar medium, and cultured at 37° C. for 16 hours. Then, the obtained colony was inoculated to LBA medium and cultured at 37° C. for 16 hours, followed by prN-SOR extraction using AxyPrep Plasmid Miniprep kit. Then, 2.5 μL (15 U/μL) of SalI, 2.5 μL (15 U/μL) of HindIII, 5.0 μL of 10×H buffer, and 10 μL of prN-SOR/pET-22b(+) Vector (Novagen) were each brought up to a constant volume of 50 μL with ultrapure water and incubated at 37° C. for 3 hours. Each restriction enzyme digestion product was subjected to agarose electrophoresis, and bands of rN-SOR (948 bp, insert) and pET-22b(+) Vector (5493 bp, vector) were excised and purified. Light absorption at 260 nm was measured to confirm concentrations. Then, the vector and the insert were mixed at vector:insert=1:3 to prepare a DNA solution. Subsequently, Ligation Mix and the DNA solution were mixed at Ligation Mix:DNA solution=1:1 and incubated at 16° C. for 3 hours for ligation reaction.

NEB® 10-beta Competent E. coli was transformed with the ligation product, and the obtained E. coli strain was inoculated to LBA agar medium and cultured, followed by plasmid DNA extraction using AxyPrep Plasmid Miniprep kit. SHuffle T7 Express LysY Competent E. coli (NEB) was transformed with the obtained plasmid DNA. The expression strain thus obtained was suspended in 80% glycerol and preserved at −80° C.

The expression strain was smeared over the surface of LBA agar medium and cultured at 30° C. for approximately 16 hours. The obtained colony was inoculated to liquid LBA medium and shake-cultured at 30° C. for 16 hours. Then, IPTG (final concentration: 0.4 mM) was added thereto, followed by shake culture at 30° C. for 4 hours. The obtained cultures were collected by centrifugation. The pellets were suspended in a homogenization buffer (50 mM Tris-HCl, pH 7.5/150 mM NaCl) in an amount of 1/20 times the culture solution and then subjected to repetitive sonication. The suspension was frozen and thawed and then centrifuged again to obtain a soluble fraction and a precipitated fraction.

The precipitated fraction was suspended in a 20-fold amount of 4% octyl phenol ethoxylate and 50 mM Tris-HCl, pH 9.0/0.1 M NaCl and shaken at room temperature for 30 minutes. Then, precipitates were recovered by centrifugation at 8000 rpm for 20 minutes. This operation was repeated four times. Then, the precipitated fraction was suspended in a 20-fold amount of ultrapure water, and precipitates were recovered by centrifugation at 8000 rpm for 10 minutes. This operation was repeated four times to remove octyl phenol ethoxylate. Next, the resulting precipitates were suspended in an approximately 20-fold amount of 8 M urea and 50 mM Tris-HCl, pH 9.0/0.1 M NaCl and shaken overnight at room temperature, and a supernatant was recovered by centrifugation. This supernatant was added to Ni column (Profinity IMAC Resin, Ni-Charged, Bio-Rad Laboratories, Inc.). Elution was performed first with 8 mL of 8 M urea and 50 mM Tris-HCl, pH 9.0/0.5 M NaCl and subsequently with 8 mL of 8 M urea, 50 mM Tris-HCl, pH 9.0/0.5 M NaCl, and 50 mM imidazole. Final elution was performed with 4 mL of 8 M urea, 50 mM Tris-HCl, pH 9.0/0.5 M NaCl, and 0.5 M imidazole. Each fraction was separated by 4 mL and subjected to SDS-PAGE to recover fractions containing the band of interest. Then, imidazole was removed by centrifugal ultrafiltration, and the internal solution was recovered with 1 mL of 8 M urea, 50 mM Tris-HCl, pH 9.0/0.1 M NaCl, and 10 mM DTT.

The sample was put in a cellulose tube (UC36-32-100, molecular weight cut-off: 14000, pore size: 50 angstroms, Sanko Junyaku Co., Ltd.) and dialyzed for 5 days under conditions of 20° C. and 4° C. for refolding. The refolding buffer used was 50 mM Tris-HCl, pH 9.0/0.1 M NaCl and replaced with a fresh one once a day. After dialysis for 5 days, and the internal dialysate was recovered and centrifuged at 8000 rpm for 10 minutes. Then, the supernatant was used as an expressed protein sample after refolding.

Plasmid DNA (4 ng) and EDTA (12.5 mM) were added to and mixed with the expressed protein (1 mg/mL) or a Tris-HCl buffer (pH 8.3) containing 0.1 M NaCl. The mixture was left at room temperature for 5 hours, separated by agarose electrophoresis, stained with ethidium bromide (15 min), and subsequently observed under UV light (260 nm). The results of FIG. 29 show that the N-terminal moiety of SOR cleaved DNA in a metal ion-dependent manner.

### [Example 9] Intracellular Translocation of Toxic Component

Whether SOR could translocate into cells and further into the nuclei was studied using fluorescently labeled SOR. The sample solution containing SOR after hydrophobic column purification in Example 4 was dialyzed against 0.1 M sodium carbonate (pH 9.4) for buffer replacement, and 30 μL of FITC-I (fluorescein-4-isothiocyanate, Dojindo Laboratories Co. Ltd., 1 mg/mL in DMSO) was added to 300 μL of the solution and reacted at 4° C. for 8 hours to obtain FITC-labeled SOR. Also, BSA labeled with FITC in the same manner as above was used as a control.

(1) Intracellular Translocation into Ba/F3 Cell

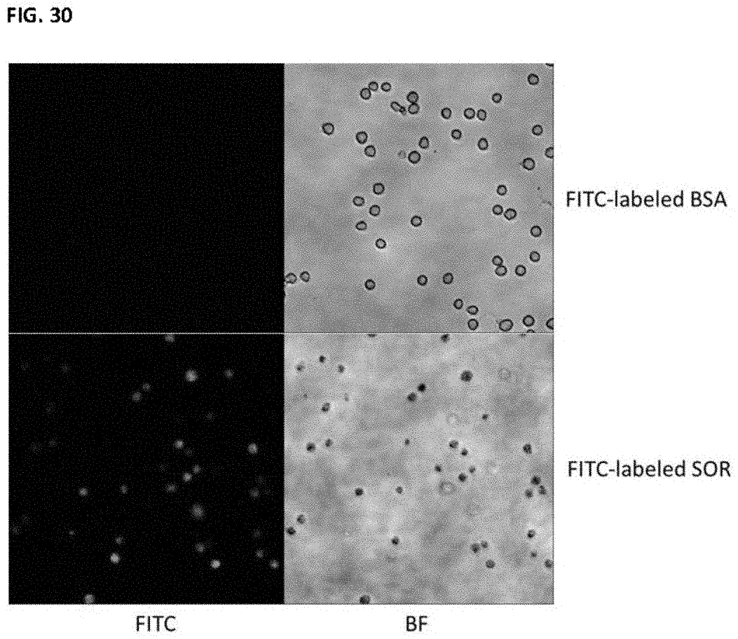

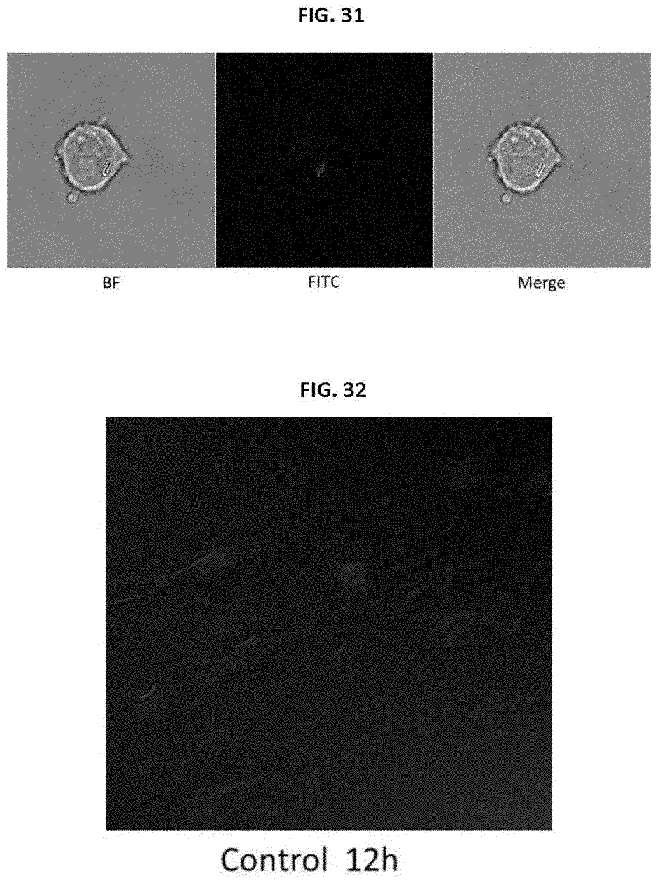

FITC-labeled SOR or FITC-labeled BSA was added in an amount of 18 μg/mL or 0.56 μg/mL in terms of protein to RPMI1640 medium containing 10% FBS and containing Ba/F3 cells at a density of 10000 cells/mL, and incubated at 37° C. for 22 hours under 5% CO₂. Then, the cells were washed with PBS and fixed in 4% PFA and 4% sucrose/PBS(−) at room temperature for 10 minutes. Subsequently, the cells thus fixed were observed under a confocal laser microscope TCS-SP5 (Leica Camera AG). The results of FIGS. 30 and 31 show that FITC was localized in the cells.

(2) Intracellular Translocation into HeLa Cell

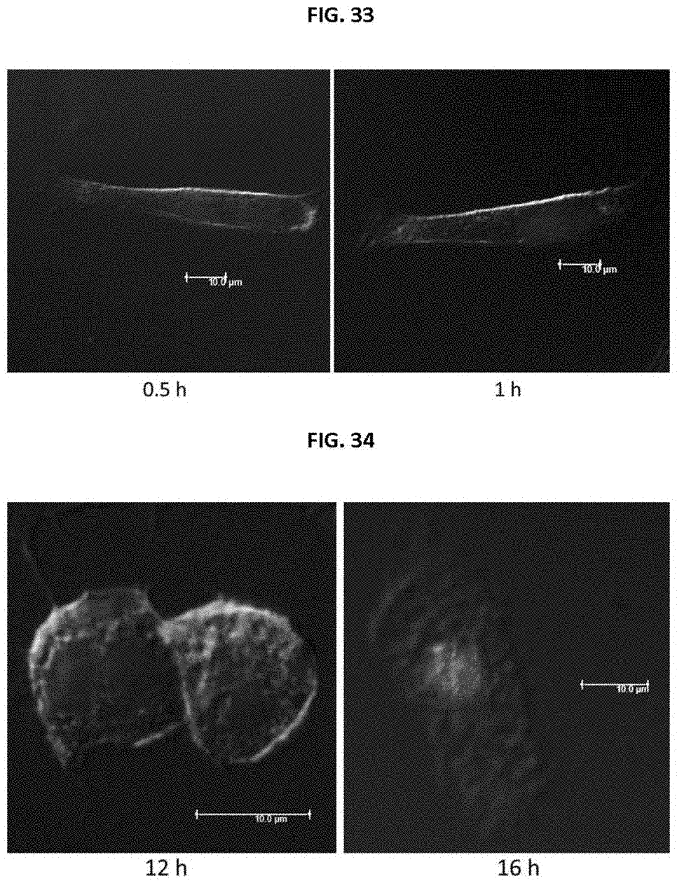

The same experiment as in the section (1) was also conducted for HeLa cells (derived from human uterine cervix cancer). A HeLa suspension prepared at approximately 2.2×10⁴ cells/mL was added at 270 μL/well to 8-well chamber cover glass (Eppendorf) to make approximately 6.0×10³ cells/well, followed by preculture at 37° C. for 24 hours in a 5% CO₂ incubator. Then, 30 μL of the FITC-SOR sample (final concentration: 18 μg/well) was added to each well and incubated at 37° C. in a 5% CO₂ incubator. The cells thus supplemented with the sample was subjected to fixing and permeabilization treatment over each time period from 30 minutes to 16 hours, and stained with DAPI. Then, a mount was prepared. The mount was observed under a confocal laser microscope (TCS-SP5), and the intracellular localization of FITC fluorescence was observed at each of the time periods after sample addition. The results are shown in FIGS. 32 to 34. The fluorescence of FITC was mostly localized around the cell membrane 30 minutes after addition, and some SOR molecules translocated into the cytoplasm 1 hour after addition. The localization of FITC fluorescence was consistent with the localization of DAPI fluorescence 12 hours or later thereafter. Thus, the intracellular translocation of SOR was confirmed.

The results described above suggested that: SOR has a structure similar to that of AB toxin (Odumosu et al., supra) constituted by an A subunit involved in toxicity and a B subunit involved in intracellular translocation; and the C-terminal moiety probably corresponding to the B subunit contributes to intracellular translocation and further to nuclear translocation. The results described above also indicate that SOR, particularly, its C-terminal moiety, is capable of functioning as a cellular and nuclear translocation carrier of various substances, in cooperation with the size of its molecular weight.

Those skilled in the art should understand that many various modifications can be made without departing from the spirit of the present invention. Thus, it should be understood that the embodiments of the present inventions described herein are merely given for illustrative purposes and are not intended to limit the scope of the present invention.

1. A polypeptide selected from
(a) a polypeptide comprising the amino acid sequence of SEQ ID NO: 1,
(b) a polypeptide that comprises an amino acid sequence containing a mutation of one or more amino acids in the amino acid sequence of SEQ ID NO: 1, and has the function of delivering a substance into a cell,
(c) a polypeptide that comprises an amino acid sequence having 80% or higher sequence identity to the amino acid sequence of SEQ ID NO: 1, and has the function of delivering a substance into a cell,
(d) a polypeptide that comprises an amino acid sequence encoded by a polynucleotide hybridizing under highly stringent conditions to a polynucleotide encoding the amino acid sequence of SEQ ID NO: 1, and has the function of delivering a substance into a cell,
(e) a polypeptide comprising the amino acid sequence of SEQ ID NO: 2,
(f) a polypeptide that comprises an amino acid sequence containing a mutation of one or more amino acids in the amino acid sequence of SEQ ID NO: 2, and has the function of degrading a nucleic acid molecule,
(g) a polypeptide that comprises an amino acid sequence having 80% or higher sequence identity to the amino acid sequence of SEQ ID NO: 2, and has the function of degrading a nucleic acid molecule,
(h) a polypeptide that comprises an amino acid sequence encoded by a polynucleotide hybridizing under highly stringent conditions to a polynucleotide encoding the amino acid sequence of SEQ ID NO: 2, and has the function of degrading a nucleic acid molecule,
(i) a polypeptide comprising the amino acid sequence of SEQ ID NO: 3,
(j) a polypeptide that comprises an amino acid sequence containing a mutation of one or more amino acids in the amino acid sequence of SEQ ID NO: 3, and has the function of degrading a nucleic acid molecule and the function of translocating into the nucleus,
(k) a polypeptide that comprises an amino acid sequence having 80% or higher sequence identity to the amino acid sequence of SEQ ID NO: 3, and has the function of degrading a nucleic acid molecule and the function of translocating into the nucleus, and
(l) a polypeptide that comprises an amino acid sequence encoded by a polynucleotide hybridizing under highly stringent conditions to a polynucleotide encoding the amino acid sequence of SEQ ID NO: 3, and has the function of degrading a nucleic acid molecule and the function of translocating into the nucleus wherein the polypeptide (a) to (d) optionally have a function of delivering a substance into the nucleus, and
wherein the cell is optionally a mammalian cell.

2. (canceled)

3. (canceled)

4. A complex of at least one of the polypeptide (a) to (d) according to claim 1 and a substance that functions intracellularly,
wherein the polypeptide and the substance that functions intracellularly are optionally bound through a covalent bond and/or a non-covalent bond, and
wherein the substance that functions intracellularly is optionally selected from a toxin, an enzyme, a cell growth-inhibiting substance, an inhibitory nucleic acid molecule, a genome editing molecule, an antibody or an antigen binding fragment thereof, a signal transduction-regulating substance, a transcriptional factor, a gene and a label.

5. (canceled)

6. (canceled)

7. A fusion polypeptide comprising, in a single molecule, at least one of the polypeptide (a) to (d) according to claim 1 and a polypeptide that functions intracellularly.

8. A composition comprising the polypeptide according to claim 1.

9. A method of treating a disease that is improved by the delivery of a substance that functions intracellularly into a cell, comprising administering the complex according to claim 4 to a subject in need thereof.

10. A method of delivering a substance that functions intracellularly into a cell, comprising contacting a complex of the substance and at least one of the polypeptide (a) to (d) and (i) to (l) according to claim 1 with the cell.

11. (canceled)

12. A complex of at least one of the polypeptide (e) to (h) according to claim 1 and a substance that promotes the translocation of the polypeptide into a cell, wherein the polypeptide and the substance that promotes the translocation of the polypeptide into a cell are optionally bound through a covalent bond and/or a non-covalent bond, and wherein the substance that promotes the translocation of the polypeptide into a cell is optionally selected from (i) a polypeptide comprising the amino acid sequence of SEQ ID NO: 1, (ii) a polypeptide that comprises an amino acid sequence containing a mutation of one or more amino acids in the amino acid sequence of SEQ ID NO: 1, and has the function of delivering a substance into a cell, (iii) a polypeptide that comprises an amino acid sequence having 80% or higher sequence identity to the amino acid sequence of SEQ ID NO: 1, and has the function of delivering a substance into a cell, (iv) a polypeptide that comprises an amino acid sequence encoded by a polynucleotide hybridizing under highly stringent conditions to a polynucleotide encoding the amino acid sequence of SEQ ID NO: 1, and has the function of delivering a substance into a cell, (v) a cell-penetrating peptide, (vi) a cell binding domain of a proteinous toxin, (vii) a virus vector and (viii) a non-viral vector.

13. (canceled)

14. (canceled)

15. A fusion polypeptide comprising, in a single molecule, at least one of the polypeptide (e) to (h) according to claim 1 and a polypeptide that promotes the translocation of the polypeptide (e) to (h) into a cell.

16. A method of treating a disease that is improved by the degradation of a nucleic acid molecule, comprising administering at least one of the polypeptide (e) to (l) according to claim 1 to a subject in need thereof.

17. A method of degrading a nucleic acid molecule, comprising-the-step of allowing at least one of the polypeptide (e) to (l) according to claim 1 to act on the nucleic acid molecule.

18. (canceled)

19. A complex of at least one of the polypeptide (i) to (l) according to claim 1 and a targeting molecule.

20. A composition comprising the complex according to claim 19.

21. A method of damaging a cell, comprising contacting at least one of the polypeptide (i) to (l) according to claim 1 with the cell.

22. A polynucleotide encoding the polypeptide according to claim 1.

23. A vector comprising the polynucleotide according to claim 22.

24. A transformed cell comprising the polynucleotide according to claim 23.

25. A method of producing the polypeptide according to claim 1, comprising culturing the transformed cell comprising a polynucleotide encoding the polypeptide under conditions suitable for the expression of the polypeptide.
